(19) United States
(12) Patent Application Publication
(10) Pub. No.: US 2026/0325593 A1
(43) Pub. Date: Oct. 22, 2026

(54) AMANITA MUSCARIA VAPORIZER FORMULATIONS AND DEVICES

(71) Applicant: Psyched Wellness Ltd., Toronto (CA)

(72) Inventors: Jeffrey Stevens, Toronto (CA); Brian Tancowny, Beaumont (CA)

(21) Appl. No.: 19/100,564

(22) PCT Filed: Aug. 1, 2023
(86) PCT No.: PCT/IB2023/057813

§ 371 Date: Feb. 1, 2025

Related U.S. Application Data
(60) Provisional application No. 63/394,250, filed on Aug. 1, 2022.

(51) Int. Cl. A61K 36/07; A61K 9/00; A61K 9/08

(52) U.S. Cl. CPC A61K 36/07; A61K 9/0073; A61K 9/08; A61K 2236/15; A61K 2236/17; A61K 2236/331; A61K 2236/51

(57) ABSTRACT

Provided herein are vaporizer formulations comprising fungal material from Amanita muscaria and/or an extract thereof, as well as devices comprising the same. Also provided are methods of using the disclosed formulations and devices to modulate neurotransmission, improve mental and physical health and wellness, and treat medical conditions.

### CROSS-REFERENCE

Priority is claimed under PCT Art. 8(1) and Rule 4.10 to U.S. Prov. App. No. 63/394,250, filed Aug. 1, 2022, and assigned to the applicant of the present application, and incorporated by reference for all purposes as if fully set forth herein.

### FIELD OF THE INVENTION

Provided herein are vaporizer formulations comprising fungal material from Amanita muscaria and/or extract thereof, as well as devices comprising the same. Also provided are methods of using the disclosed formulations and devices to modulate neurotransmission, improve mental and physical health and wellness, and treat medical conditions.

### BACKGROUND OF THE INVENTION

Mushrooms of the Amanita genus have long been a part of the human diet, and certain Amanita species are also known for their psychoactive properties. Amanita muscaria, also known as the fly agaric, is a mycorrhizal basidiomycete fungus native to temperate and boreal regions of the Northern Hemisphere. Ingestion of the Amanita muscaria by indigenous peoples of Siberia and Mexico is a centuries-old practice of cultural and religious significance. Amanita muscaria use for spiritual and recreational purposes continues in the present day. However, injury and even deaths have occurred as a result of inorganic and organic toxins present in Amanita muscaria mushroom biomass, including ibotenic acid, muscarine, and heavy metals. There is therefore a need for formulations that allow for the safe use of Amanita muscaria, including extracts and compounds derived therefrom. Provided herein are such formulations, which are capable of conferring benefits to mental and physical health and wellness, conveying the restorative and healing elements of Amanita muscaria, and having advantages and improvements as will become readily apparent through the disclosure below.

### INCORPORATION BY REFERENCE

Each patent, publication, and non-patent literature cited in the application is hereby incorporated by reference in its entirety as if each was incorporated by reference individually. However, where such reference is made, and whether to patents, publications, non-patent literature, or other sources of information, it is for the general purpose of providing context for discussing features of the present invention. Accordingly, unless specifically stated otherwise, reference is not to be construed as an admission that the document or underlying information, in any jurisdiction, is prior art or part of the common general knowledge in the art.

### BRIEF SUMMARY OF THE INVENTION

The following presents a simplified summary of some embodiments of the invention in order to provide a basic understanding thereof. This summary is not an extensive overview of the invention. It is not intended to identify key or critical elements of the invention, or to delineate its full scope. Its sole purpose is to present some embodiments of the invention in a simplified form as a prelude to the more detailed description that is presented later.

In a first aspect, provided is a vaporizable formulation comprising: (a) an Amanita muscaria extract; (b) a base liquid; and (c) optionally, a flavorant; wherein the mass ratio of muscimol:ibotenic acid in the Amanita muscaria extract is greater than about 10:1.

In some embodiments (equivalently, and as shorthand, “in embodiments”), the muscimol:ibotenic acid ratio is greater than about 100:1. In embodiments, the muscimol:ibotenic acid ratio is greater than about 1000:1. In embodiments, the muscimol:ibotenic acid ratio is between about 10:1 and 50:1. In embodiments, the muscimol:ibotenic acid ratio is between about 10:1 and 30:1. In embodiments, the muscimol:ibotenic acid ratio is about 12.5:1. In embodiments, the muscimol:ibotenic acid ratio is about 25:1.

In some embodiments, the Amanita muscaria extract comprises less than about 550 μg/g of muscarine. In some embodiments, the Amanita muscaria extract comprises less than about 40 μg/g of stizolobinic acid. The vaporizable formulation of claim 1, wherein the Amanita muscaria extract comprises less than about 40 μg/g or 20 μg/g of stizolobinic acid.

In some embodiments, the Amanita muscaria extract comprises less than about 0.05 ppm, 0.04 ppm, or 0.03 ppm of arsenic. In embodiments, the Amanita muscaria extract comprises less than about 0.30 ppm, 0.25 ppm, 0.20 ppm, 0.15 ppm, 0.10 ppm, or 0.09 ppm of cadmium. In embodiments, the Amanita muscaria extract comprises less than about 0.15 ppm, 0.10 ppm, 0.05 ppm, or 0.02 ppm of mercury. In embodiments, the Amanita muscaria extract comprises less than about 0.09 ppm, 0.05 ppm, 0.04 ppm, 0.03 ppm, or 0.02 ppm of lead.

In some embodiments, the vaporizable formulation is produced by: (a) suspending an Amanita muscaria extract in a solvent; (b) mixing the suspension; (c) isolating the supernatant from the suspension, thereby producing an extract solution; (d) evaporating the extract solution to provide a residue; and (e) dissolving the residue in a base liquid to produce the vaporizable formulation. In embodiments, the Amanita muscaria extract is a ground powder extract. In embodiments, the solvent is an organic solvent. In embodiments, the solvent is a methanol. In embodiments, the supernatant comprises centrifuging of the suspension. In embodiments, evaporating the extract solution comprises heating the extract solution.

In some embodiments, the vaporizable formulation is produced by: (a) performing an aqueous extraction of Amanita muscaria mushroom biomass in the presence of heat to produce the extract; (b) reducing the pH of the extract; (c) concentrating the extract; and (d) dissolving the extract in a base liquid to produce the vaporizable formulation. In embodiments, the aqueous extraction is performed in water heated to between about 70° C. and 100° C. In embodiments, the aqueous extraction is performed in water heated to between about 80° C. In embodiments, the pH is reduced to between about 2.5 and about 3.0. In some embodiments, the base liquid comprises propylene glycol, vegetable glycerin, or a combination thereof.

In some embodiments, the mass ratio of the Amanita muscaria extract to the base liquid is about 1 part extract to about 10 parts base liquid. In embodiments, the vaporizer formulation comprises between about 40% v/v to 90% v/v of the base liquid. In embodiments, the vaporizer formulation comprises between about 40% v/v and about 90% v/v of propylene glycol. In embodiments, the vaporizer formulation comprises between about 40% v/v and about 90% v/v of vegetable glycerin. In embodiments, the vaporizer formulation comprises between about 0.1% v/v and about 10% v/v of the flavorant. In embodiments, the vaporizer formulation comprises between about 0.1% v/v and about 5% v/v of the flavorant. In embodiments, the vaporizer formulation comprises between about 0.1% v/v and about 1% v/v of the flavorant.

In another aspect, provided is a method of producing the vaporizable formulation of any of the disclosed embodiments, comprising: (a) suspending an Amanita muscaria extract in a solvent; (b) mixing the suspension; (c) isolating the supernatant from the suspension, thereby producing an extract solution; (d) evaporating the extract solution to provide a residue; and (e) dissolving the residue in a base liquid to produce the vaporizable formulation. In some embodiments, the Amanita muscaria extract is a ground powder extract. In embodiments, the solvent is an organic solvent. In embodiments, the solvent is a methanol. In embodiments, isolating the supernatant comprises centrifuging of the suspension. In embodiments, evaporating the extract solution comprises heating the extract solution. In embodiments, the method further comprises dissolving a flavorant in the base liquid.

In another aspect, provided is a method of producing the vaporizable formulation of any of the disclosed embodiments, comprising: (a) performing an aqueous extraction of Amanita muscaria mushroom biomass in the presence of heat to produce the extract; (b) reducing the pH of the extract; (c) concentrating the extract; and (d) dissolving the extract in a base liquid to produce the vaporizable formulation. In some embodiments, the aqueous extraction is performed in water heated to between about 70° C. and 100° C. In embodiments, the aqueous extraction is performed in water heated to between about 80° C. In embodiments, the pH is reduced to between about 2.0 and about 4.0. In embodiments, the pH is reduced to between about 2.5 and about 3.0. In embodiments, the method further comprises dissolving a flavorant in the base liquid.

In another aspect, provided is a method of modulating neurotransmission in a subject, comprising administering to the subject the vaporizable formulation of any of the disclosed embodiments. Also provided is the vaporizable formulation of any of the disclosed embodiments, for use in modulating neurotransmission. In some embodiments, modulating neurotransmission comprises modulating one or more of gabaminergic, glutaminergic, and cholinergic neurotransmission.

Also provided is the use of the vaporizable formulation of any of the disclosed embodiments, for the manufacture of a medicament for the treatment of a medical condition.

In yet another aspect, provided is a method of treating a medical condition in a subject in need of such treatment, comprising administering to the subject the vaporizable formulation of any of the disclosed embodiments. In embodiments, the medical condition is a disorder linked to dysregulation or inadequate functioning of gabaminergic, glutaminergic, or cholinergic neurotransmission. In embodiments, the medical condition is a mental, behavioral, or neurodevelopmental disorder. In embodiments, the mental, behavioral, or neurodevelopmental disorder is a neurodevelopmental disorder, schizophrenia or another primary psychotic disorder, catatonia, a mood disorder, an anxiety or fear-related disorders, an obsessive-compulsive or related disorder, a disorder specifically associated with stress, a dissociative disorder, a feeding or eating disorder, an elimination disorder, a disorder of bodily distress or bodily experience, a disorder due to substance use or addictive behavior, an impulse control disorder, a disruptive behavior or dissocial disorder, a personality disorder, a paraphilic disorder, a factitious disorder, a neurocognitive disorder, a mental or behavioral disorder associated with pregnancy, childbirth or the puerperium, a sleep-wake disorder, or a sexual dysfunction.

In embodiments, the medical condition is pain or a pain disorder, inflammation or an inflammatory disorder, an immune or autoimmune disorder, or a sleep disorder. In embodiments, the pain disorder is any of arthritis, allodynia, atypical trigeminal neuralgia, trigeminal neuralgia, somatoform disorder, hypoesthesis, hyperalgesia, neuralgia, heuritis, neurogenic pain, analgesia, anesthesia dolorosa, causalgia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer. In some embodiments, the autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune hemolysis anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopenia purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, systemic lupus erythematosus, morphea, multiple sclerosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo. In some embodiments, the autoimmune disorder is a systemic autoimmune disorder, including systemic lupus erythematosus (SLE), Sjogren's syndrome, scleroderma, rheumatoid arthritis, and polymyositis. In embodiments, the sleep disorder is any of an insomnia, a hypersomnia, a parasomnia, and a disorder of sleep-wake schedule

Also provided is a method of improving health and wellness in a subject, comprising administering to the subject the vaporizable formulation of any of the disclosed embodiments. In some embodiments, improving health and wellness comprises any of reducing stress, easing muscular tension, promoting restorative sleep, soothing the body, calming the mind, reducing physical distress, reducing anxiety, and inducing euphoria. In embodiments, improving health and wellness comprises treating a medical condition.

In another aspect, provided is a vaporizer device, comprising: (a) a mouthpiece; (b) a heating element; (c) a power source; and (d) a chamber comprising the vaporizable formulation of any of the disclosed embodiments. In some embodiments, the heating element is capable of heating the vaporizable formulation to a set temperature. In embodiments, the set temperature is sufficient to vaporize the vaporizable formulation. In embodiments, the power source powers the heating element. In embodiments, the heating element comprises a wick element. In embodiments, the chamber comprises a first end configured to attach to the mouthpiece. In embodiments, the chamber comprises a second end configured to attach to the heating element and the power source. In embodiments, the chamber is refillable. In embodiments, the chamber is removable. In embodiments, the vaporizer device further comprises a means for controlling the device. In embodiments, the means for controlling the device comprises switches, buttons, touch sensors, or a combination thereof. In embodiments, the vaporizer device further comprises a means for displaying information. In embodiments, the means for displaying information comprises a screen, a number of lights, or a combination thereof. In embodiments, the vaporizer device further comprises a number of sensors configured to sense temperature, pH, volume, the means for controlling the device, or a combination thereof.

These and other embodiments, objects, features, and advantages of the invention may be more clearly understood and appreciated from a review of the detailed description of disclosed embodiments and examples, and by reference to the appended claims. This summary has been made with the understanding that it is to be considered as a brief and general synopsis of only some of the objects and embodiments disclosed herein, is provided solely for the benefit and convenience of the reader, and is not intended to limit in any manner the scope, or range of equivalents, to which the appended claims are lawfully entitled.

### BRIEF SUMMARY OF THE DRAWINGS

To further clarify various aspects of the invention, a more particular description of the invention will be rendered by reference to certain exemplary embodiments thereof which are illustrated in the included figures. It should be understood and appreciated that the figures depict only illustrated embodiments of the invention and are therefore not to be considered limiting of its scope. They are simply provided as exemplary illustrations of certain concepts of some embodiments of the invention.

Certain aspects of the invention are therefore further described and explained with additional specificity and detail, but still by way of example only, with reference to the accompanying figures in which:

FIG. 1 is a flowchart of an exemplary process for producing a vaporizer formulation in accordance with a first embodiment described herein; and

FIG. 2 is a flowchart of an exemplary process for producing a vaporizer formulation in accordance with an additional embodiment described herein.

### DETAILED DESCRIPTION OF THE INVENTION

While various aspects and features of certain embodiments have been summarized above, the detailed description illustrates several embodiments in further detail to enable one of skill to practice such embodiments. The described examples are provided for illustrative purposes and are not intended to limit the scope of the invention or its applications. This disclosure is not limited to particular embodiments described. It will be understood that many modifications, substitutions, changes, and variations in the described embodiments, applications, and details of the invention illustrated herein can be made by those skilled in the art without departing from the spirit of the invention, or the scope of the invention as described in the appended claims. The headings within this document are being utilized only to expedite its review by a reader. They should not be construed as limiting the present invention in any manner.

Among the various aspects of the invention are vaporizer formulations comprising fungal material, including mushrooms, such as Amanita muscaria mushrooms, fungal extracts such as Amanita muscaria extracts, as well as compounds from such extracts, including muscimol and muscarine, chambers and devices comprising the same, and methods of their use for the general improvement of mental and physical health and wellness, and for treating diseases and disorders.

The scope of the invention includes all embodiments and formulations or devices thereof and it will be understood that many modifications, substitutions, changes, and variations in the described embodiments, applications, and details illustrated herein can be made by those skilled in the art without departing from the spirit of the invention, or the scope of the invention as described in the appended claims.

### A. General Definitions and Terms

When introducing elements of the present invention or the embodiments thereof, the articles “a,” “an,” “the,” and “said” are intended to mean that there are one or more of the elements. Any reference to an element in the singular is therefore not intended to mean “one and only one” unless specifically so stated, but rather “one or more;” therefore, the term “or” standing alone, unless context demands otherwise, shall mean the same as “and/or.” The terms “comprising,” “including,” “such as,” and “having” are also intended to be inclusive and not exclusive (i.e., there may be other elements in addition to the recited elements). Thus, for example, the terms “including,” “may include,” and “include,” as used herein mean, and are used interchangeably with, the phrase “including but not limited to.” The word “exemplary” is used herein to mean “serving as an example, instance, or illustration.” Any aspect, embodiment, process, or implementation described as “exemplary” is thus not to be construed as necessarily preferred or advantageous over others.

Unless otherwise stated, all measurements, values, ratings, positions, magnitudes, sizes, locations, orientations, configurations, and other specifications that are set forth (either expressly or impliedly) in this specification, including in the figures and in the claims that follow, are approximate, and not exact. They are intended to have a reasonable range that is consistent with the functions to which they relate and with what is customary in the art to which they pertain.

Unless otherwise stated, all numbers expressing quantities of ingredients, properties such as concentration, and so forth, used to describe and claim certain embodiments of the invention are to be understood as being modified in some instances by the term “about.” Where the term “about” is used to modify a value, it means that a range of values that includes the specified value is also encompassed, which a person of ordinary skill would consider reasonably similar to the specified value. Where numerical values may be modified by the term “about,” it will be understood that in some embodiments, such values are modified by “about,” but in other embodiments, such values are within a further degree of precision. In embodiments, “about” means within a standard deviation using measurements generally acceptable in the art. In embodiments “about” means within manufacturing tolerances. In other embodiments, “about” means a range extending to +/−10% of the specified value. Accordingly, In embodiments, the numerical parameters set forth in the written description and attached claims are approximations that can vary depending upon the desired properties sought to be obtained by a particular embodiment.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable, taking into consideration production variances and manufacturing tolerances and the like. Accordingly, the numerical values presented In embodiments of the invention may contain certain errors necessarily resulting from the standard deviation found in their respective testing measurements.

Generally, the nomenclature used and procedures performed herein are those known in field(s) relating to that of one or more aspects of the invention, and are those that will be well-known and commonly employed in such field(s). Standard techniques and procedures will be those generally performed according to conventional methods in the art. Unless defined otherwise, all technical and scientific terms herein have the meaning as commonly understood by a person having ordinary skill in the art to which this invention belongs, who as a shorthand may be referred to simply as “one of skill.” Further definitions that may assist the reader in understanding the disclosed embodiments are as follows; however, it will be appreciated that they will not be used to limit the scope of the invention, which shall be properly interpreted and understood by reference to the full specification (as well as any plain meaning known to one of skill in the relevant art) in view of the language used in the appended claims. That is, the terminology used herein is for the purpose of describing particular embodiments only and is not intended to be limiting. Although any materials and methods similar or equivalent to those described herein can be used in the practice of the present invention, certain preferred materials and methods for some exemplary embodiments are now described.

### B. Amanita Muscaria

“Amanita muscaria” (equivalently, “A. muscaria”) will be understood to include the Amanita muscaria var. Muscaria (Euro-Asian fly agaric), Amanita muscaria var. flavivolvata (American fly agaric), Amanita muscaria var. guessowii (American fly agaric, yellow variant), and Amanita muscaria var. inzengae (“Inzenga's” fly agaric); also within the scope and spirit of the invention, and which thus shall be considered within the definition of Amanita muscaria or as equivalents thereof, are such other ibotenic acid and muscimol containing Amanita species that are not known to be deadly poisonous, as will be appreciated as within the general knowledge in the art.

Amanita muscaria is known to contain several psychoactive compounds as well as some other biologically active substances. Accordingly, chemists, pharmacologists, and ethnobotanists have deployed great efforts in attempts to resolve the chemical composition of these mushrooms and to explain the physiological effects attributed to them for over 100 years. Muscimol (5-(aminomethyl)-1,2-oxazol-3 (2H)-one) is one of the main psychoactive components of A. muscaria. Muscimol is known to be an agonist for GABAA receptors (Johnston, Eurochem Res. 2014; 39 (10): 1942-7). When binding to a GABAA receptor, muscimol activates the receptor, causing anxiolytic, anticonvulsant, amnesic, sedative, hypnotic, euphoriant, and muscle relaxant properties. Muscimol also may have hallucinogenic effects.

Another psychoactive component of Amanita muscaria is ibotenic acid ((S)-2-amino-2-(3-hydroxy-isoxazol-5-yl)), a conformationally-restricted analogue of the neurotransmitter glutamate that acts as a non-selective glutamate receptor agonist. Ibotenic acid can also act as a neurotoxin under certain conditions; for example, in scientific research, cranially injected ibotenic acid has been employed as a “brain-lesioning agent.” Ibotenic acid can be converted to muscimol by a chemical process known as decarboxylation. “Decarboxylation” refers to a chemical reaction that removes a carboxyl group and releases carbon dioxide (CO₂), thereby replacing a carboxyl group (—COOH) with a hydrogen atom (H); e.g., as RCO₂H→RH+CO₂.

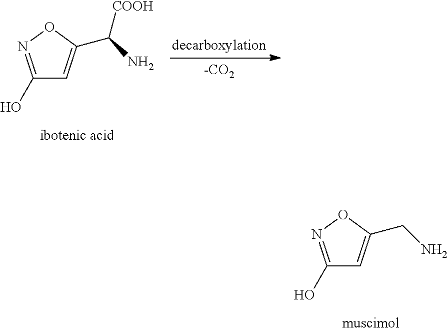 [SMILES: N[C@H](C(=O)O)c1cc(O)no1 ; NCc1cc(O)no1]

In typical samples, Amanita muscaria contains more ibotenic acid than muscimol. At least some of the ibotenic acid in Amanita muscaria is converted by decarboxylation to muscimol in the acid environment of the stomach. Ibotenic acid therefore serves as a prodrug to muscimol. However, if too much ibotenic acid is ingested, it can cause stomach irritation, nausea, diarrhea, sweating and salivation, lethargy and drowsiness, ataxia, and other somatic symptoms, as well as psychological symptoms such as confusion, euphoria, visual and auditory hallucinations, sensations of floating, distortions of space and time, and retrograde amnesia (Moss et al. Clin Toxicol (Phila). 2019; 57(2):99-103).

Muscarine (2,5-anhydro-1,4,6-trideoxy-6-(trimethyl-ammonio)-D-ribo-hexitol) is another component of A. muscaria, typically found in lower quantities than ibotenic acid and muscimol. Muscarine is a nonselective agonist of the muscarinic acetylcholine receptors (Broadley et al., Molecules, 2001; 6(3):142-193). Muscarine may be toxic in concentrations found in certain mushroom species, but not typically in the concentrations found in Amanita muscaria.

### c. Amanita Muscaria Extracts

Despite the documented benefits of consuming A. muscaria mushrooms, widespread acceptance and adoption by consumers has been limited, in part, by the variability in potency of the mushrooms themselves, as well as concerns about potential neurotoxicity associated with excessive ibotenic acid, and contamination by heavy metals and other pollutants present in soil. Many of these concerns can be addressed or entirely alleviated by consuming the beneficial components of Amanita muscaria in the form of an extract, which can be standardized to a fixed concentration or a fixed ratio of active agents (e.g., muscimol, muscarine, ibotenic acid) to enable predictable dosing.

Extracts such as disclosed herein offer additional advantages: they can have improved shelf stability (especially when preservatives are incorporated into the extract), they can improve consumer or patient experience (e.g., by providing a more flavorful product), they can be purified and subjected to chemical analysis to ensure they are free of toxins and pollutants, and they can be more easily incorporated into other consumable products, such as inhalable or vaporizable formulations (e.g., for use in a vaporizer). Indeed, in cultures in which Amanita muscaria mushrooms have a history of entheogenic use, it is common to prepare the mushrooms for consumption by boiling them in water to extract the active principles and induce (partial) decarboxylation of ibotenic acid to muscimol (Feeney, et al., J. Psychoact. Drugs 2011, 42(4), 4499-506).

A. muscaria extracts useful in the practice of the invention include extracts produced according to disclosed methods, or those disclosed in Applicant's prior applications published as WO2022/187974A1 and WO2023/015395A1, each of which is hereby incorporated by reference in its entirety, as if fully set forth herein. In some embodiments, the extract will be the one styled here and in the field by Applicant as AME-1@ or herein “AME-1” (with characteristics as shown in Tables 1-7) obtained from certain exemplary methods disclosed herein. Although those of skill will recognize a variety of methods useful for the preparation of AME-1, certain methods, which are non-limiting methods of production, are described herein.

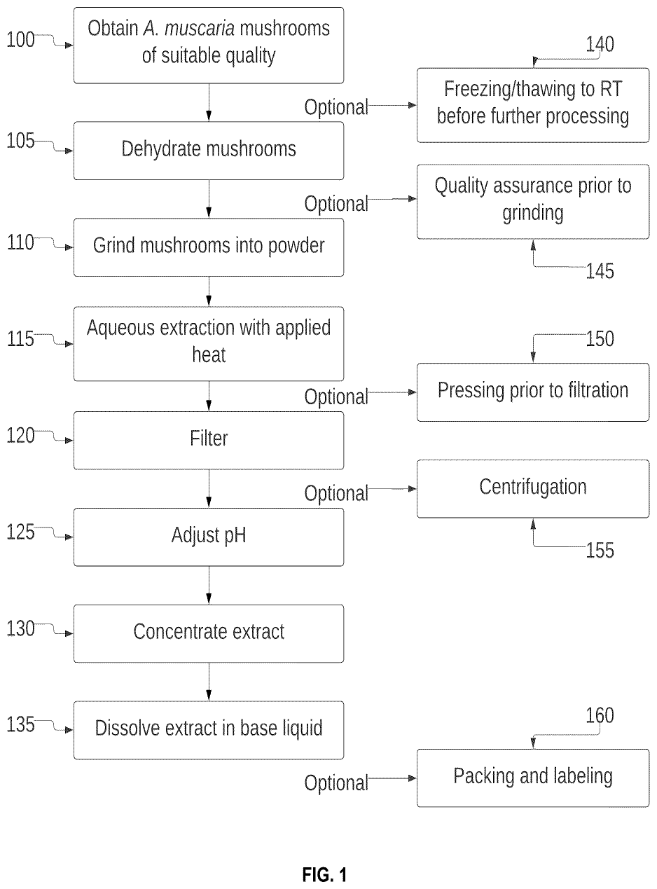

FIG. 1 is a flowchart of an exemplary extraction and concentration procedure, Amanita muscaria mushrooms (below as shorthand and for convenience, “mushrooms”) are harvested or gathered, or are otherwise obtained as fresh mushrooms. Alternatively, mushrooms may be obtained dried. Optionally, the mushrooms are inspected to ensure that they are of suitable quality (100). Mushrooms may already have been inspected at this point, rendering further inspection unnecessary, or only for spot quality control. Such inspection may identify debris, dirt, and other organic matter (which will be discarded), may be useful in confirming the correct species, and removing mistaken species, especially those that are toxic or poisonous; and/or may aid in determining general “quality,” meaning overall appearance, cleanliness, color, damage, apparent age, presence or absence of signs of worms or insects, etc. However, it should be noted, such inspection is not required and, as a result, should not be construed as limiting the invention disclosed herein. Optionally, the mushrooms may be frozen for a period of storage prior to further processing, upon which time the mushrooms may be thawed (140).

Without being bound by theory, it is generally found that mushroom caps will have the highest concentration of target compounds, and thus in certain preferred embodiments, the caps are removed from the stipes prior to processing, and only the caps are used, therefore leading to greater overall yields of muscimol, and higher potency. In some embodiments, both caps and stipes are used. In some embodiments, only stipes are used.

In some embodiments, the mushrooms utilized in a disclosed process are sufficiently dry, having a moisture content of from about 2% to about 3% moisture by weight. That said, in some embodiments, the mushrooms may have less than 2% or more than 3% moisture by weight. For example, dehydrated mushrooms may have a moisture content of from about 4% to about 7% by weight. If not sufficiently dry when harvested, the mushrooms may be dried (also termed “dehydrated”) (105) in a dehydrator or by application of heat in any conceivable method known to those of skill, including but not limited to conduction, convection, or radiation.

It will be appreciated, however, that if dried below 2%, or at too high of a temperature, muscimol and ibotenic acid could be degraded, reducing the final yield of muscimol that will be obtained by the process. The appropriate amount of drying may be determined by practice of ordinary skill for the specific embodiment of the invention being carried out. In one illustrative, non-limiting way of determining an adequate level of moisture content, mushrooms in the desired moisture range easily snap in half. Thus, in some embodiments, dehydration is often completed until the mushrooms are “cracker dry.” In some embodiments, if the mushroom is merely pliable, and not brittle, it is likely not dry enough. In some embodiments, dryness will be considered sufficient when the mushrooms will be able to be ground into a powder. That said, drying the harvested mushrooms is merely an optional step. Thus, embodiments wherein the mushrooms are not dried, or are of a moisture content outside the range disclosed above (e.g., 1%, 2%, 3%, 4%, 5%, 6%, 7%, or greater than 7%, wherein the range is inclusive and each number may be modified by the term “about”), are also encompassed by the present disclosure.

In some embodiments wherein drying is performed (105), drying may be completed at a maximum temperature of 50° C., including 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., or less than 20° C. The mushrooms may be dried in a circulated air dehydration unit, a forced air food dryer, or any other such device capable of maintaining a substantially constant temperature set by a user, wherein heating is completed via conduction (direct heating), convection (heating via continuous currents of a gas or liquid), and/or radiation (heating via absorption of heat by a cooler body from a warmer body).

Additionally, drying may, in some embodiments, last from about 24 hours to about 48 hours, including 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, and 48 hours, wherein the range is inclusive and each value will be understood as being both modified and not modified by the term “about.” Likewise, in embodiments, heating may be completed for less than 24 hours, such as 20 hours, 16 hours, 12 hours, 8 hours, or less than 8 hours; or greater than 48 hours, such as 56 hours, 60 hours, 65 hours, 72 hours, or 80 hours (inclusive).

In some embodiments, whether or not the mushrooms are dehydrated, they may be sealed in plastic bags or airtight containers and stored in darkness until processing. In such embodiments, the temperature at which the mushrooms are stored should correspond with how soon processing may take place. Generally, mushrooms are stored at between about −25° C. and about 3° C. However, but not to be bound by theory, storage at the warmer temperatures of the aforementioned range is only recommended if processing will imminently occur (such as within about 5 days) because prolonged storage at temperatures above 3° C. may result in a loss of potency and gradual destruction of the mushrooms by macroscopic and microscopic organisms. The appropriate amount of drying may be determined by one of ordinary skill. Drying the harvested mushrooms is also an optional step, and should not be construed as limiting.

In embodiments, the mushrooms, including dried mushrooms, are ground (e.g., to a powder) by a food processor, coffee grinder, blender, or similar device, including like devices of commercial or industrial scale (110). Mushrooms also may be chopped, pulverized, and/or otherwise rendered to smaller pieces or particles, by methods and devices known to those of skill. Although steps below may be described as using “ground mushrooms,” it will be appreciated that such mushrooms may be whole or in pieces or in particles of various sizes, all of which are within the scope of the methods, but will have properties that may differ according to ordinary skill, for example that smaller pieces or particles may require lower processing times, fewer steps such as fewer extraction steps, less total solvent, and/or provide an extract of greater potency.

In an optional step (145), mushrooms are subjected to quality assurance analyses prior to grinding. In embodiments, a small batch of mushrooms or the ground powder thereof (together below for convenience simply “mushrooms”) is optionally analyzed to determine whether the muscimol, muscarine, and ibotenic acid are within safety and production specifications. In addition, the batch may or may not be analyzed for heavy metal and pesticide content. The analysis may be performed by any means known to those of skill capable of completing such an analysis, e.g., high performance liquid chromatography tandem mass spectrometry (HPLC-MS/MS).

An aqueous extraction (115) may then be conducted. In some embodiments, water is obtained, the pH is determined, and is maintained at about 7, with adjustments made as necessary and as known in the art. In embodiments, the water is boiled, and the mushrooms are placed in the boiled water and stirred.

In some embodiments, the aqueous extraction is performed in water heated to between about 70° C. and 100° C. For example, in some embodiments, the temperature of the mixture of water and mushrooms is maintained at 95° C. to 100° C. In some embodiments, the temperature of the mixture of water and mushrooms is maintained at about 80° C. In some embodiments, the mixture is stirred for between about 5 minutes to about 180 minutes at between about 700 rpm and about 2500 rpm.

As it relates to stirring specifically, the mixture is stirred for between about 5 minutes to about 180 minutes, including 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or greater than 180 minutes, wherein the range is inclusive and each value may be modified by the term “about;” at between 700 rpm and 2500 rpm, including about 750 rpm, 800 rpm, 850 rpm, 900 rpm, 950 rpm, 1000 rpm, 1050 rpm, 1100 rpm, 1150 rpm, 1200 rpm, 1250 rpm, 1300 rpm, 1350 rpm, 1400 rpm, 1450 rpm, 1500 rpm, 1550 rpm, 1600 rpm, 1650 rpm, 1700 rpm, 1750 rpm, 1800 rpm, 1850 rpm, 1900 rpm, 1950 rpm, 2000 rpm, 2050 rpm, 2100 rpm, 2150 rpm, 2200 rpm, 2250 rpm, 2300 rpm, 2350 rpm, 2400 rpm, 2450 rpm, 2500 rpm, and values in between, wherein each value may be modified by the term “about.”

It will be readily appreciated that, although the length of time for stirring may differ, the duration need only be that which is sufficient to mix the solution, such that the mushroom powder and water form a substantially homogeneous slurry. In some embodiments, mixing may be performed by hand, for example by utilization of a stirring utensil, such as a whisk, a spoon, or a spatula; or using a stir plate, for example using an electronic hand mixer, using an electronic mixture having its own arm, or, in some embodiments, using an industrial vat capable of containing the mixture of the invention and possessing at least one agitation means (such as, but not limited to, paddles or arms).

In some embodiments, agitation may be completed for between about 5 minutes, to at least 180 minutes—the specific amount of time required reflecting the given embodiment being practiced. Meaning, in some embodiments, the duration of time required for agitation may be based on a standard amount of time known for that given embodiment, such as but not limited to an embodiment wherein the suggested agitation time is 60 minutes. In other embodiments, agitation may be an iterative process wherein agitation ceases when the mixture forms a substantially homogeneous slurry. In some embodiments, a substantially homogeneous slurry is characterized by substantially uniform dispersion of the mushroom particles within the water, which may be evidenced by the naked eye, and would be immediately apparent to one of skill. However, such uniform dispersion may additionally be determined electronically, using devices known to those of skill capable of determining sample variance.

As mentioned, the water is heated, in some embodiments, to between about 70° C. to about 100° C. Such temperatures aid in extracting muscimol from the tissue of the mushroom. In some embodiments, the ratio of ground mushroom caps to water may be about 1 gram of mushroom: 40 ml of water, such that 1 gram, 5 grams, 7 grams, 10 grams, 12 grams, 15 grams, 17 grams, 20 grams, 25 grams, 30 grams, 35 grams, 40 grams, 45 grams, 50 grams, 55 grams, 60 grams, 65 grams, 70 grams, 75 grams, 80 grams, 85 grams, 90 grams, 95 grams, or 100 grams of mushroom extract may be combined with 40 mL, 80 mL, 280 mL, 400 mL, 480 mL, 600 mL, 680 mL, 800 mL, 1000 mL, 1200 mL, 1400 mL, 1600 mL, 2000 mL, 2200 mL, 2400 mL, 2600 mL, 2800 mL, 3000 mL, 3200 ml, 3400 mL, 3600 mL, 3800 mL, 4000 mL, respectively. In some embodiments, the ratio of water to mushroom may be from about 5 ml of water per 1 g of mushroom (i.e., a 5:1 water:mushroom ratio), up to about 20 ml of water per 1 g of mushroom (i.e., a 20:1 water:mushroom ratio). In embodiments, the water:mushroom ratio is about 5:1, 10:1, 11:1, 12:1, 13:1, 14:1, 15:1, 16:1, 17:1, 18:1, 19:1 or 20:1, or values in between these ratios.

In some embodiments, the mushroom powder is added to water at about from 65° C. to about 150° C. In some embodiments, the water temperature is about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C. In some embodiments, the boiled water is allowed to cool to about 75° C. before adding the ground mushrooms. In some embodiments, the water is heated to about 75° C. before adding the ground mushrooms. In some embodiments, the water is maintained at a temperature of 75° C. prior to adding the ground mushrooms. In embodiments, the water is maintained at a temperature of 80° C. prior to adding the ground mushrooms.

In embodiments, the mixture is then filtered to remove solids (120). In embodiments, the filtrate may be collected in a flask. However, this is an exemplary, non-limiting embodiment, and any such container sufficient to contain the filtrate may be used for collection.

In embodiments, pressure is applied during the filtration process (150), such as with use of a fruit press. In embodiments, the mixture may be drawn through the filter(s) by vacuum filtration. In an optional embodiment, the filtrate is centrifuged to separate particulate matter and a supernatant (155).

In embodiments wherein centrifuging is conducted, it may take place for between about 1 and about 25 minutes, including 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, and values in between, wherein each value may be modified by the term “about;” at between about 2000 to about 6000 rpm, including about 2100 rpm, 2200 rpm, 2300 rpm, 2400 rpm, 2500 rpm, 2600 rpm, 2700 rpm, 2800 rpm, 2900 rpm, 3000 rpm, 3100 rpm, 3200 rpm, 3300 rpm, 3400 rpm, 3500 rpm, 3600 rpm, 3700 rpm, 3800 rpm, 3900 rpm, 4000 rpm, 4100 rpm, 4200 rpm, 4300 rpm, 4400 rpm, 4500 rpm, 4600 rpm, 4700 rpm, 4800 rpm, 4900 rpm, 5000 rpm, 5100 rpm, 5200 rpm, 5300 rpm, 5400 rpm, 5500 rpm, 5600 rpm, 5700 rpm, 5800 rpm, 5900 rpm, 6000 rpm, and values in between, wherein each value may be modified by the term “about.” In some embodiments, centrifuging may take place for about 15 minutes at about 4000 rpm at room temperature. In some embodiments, if there is a resulting pellet of non-soluble and/or fibrous material, it is discarded. The supernatant is then collected.

In some embodiments, the pH of the filtrate may be adjusted (125) to between about 5-6, or lower, by adding an acid to the flask. In some embodiments, the pH of the extract is reduced. In some embodiments, the pH of the extract is reduced to between about 2.0 and about 4.0. In some embodiments, the pH of the extract is reduced to between about 2.5 and about 3.0. In some embodiments, the acid is acetic acid, boric acid, carbonic acid, citric acid, hydrochloric acid (HCl), hydrofluoric acid (HF), nitric acid, oxalic acid, phosphoric acid, sulfuric acid, or any other compound known to those of skill capable of lowering the pH of the filtrate. In some embodiments, the acid may be HCl and the pH may be lowered to about 3.0, for example, by the addition of 1 M HCl, for example. In some embodiments, the pH may be lowered to about 2.5. Without being bound by theory, the increased acidic environment may cause ibotenic acid to convert into muscimol by decarboxylation. If the mushrooms are not sufficiently dried (to between about 2% to about 3% moisture content), a higher concentration of HCl or other acid may be needed to achieve the same degree of conversion of ibotenic acid to muscimol.

In some embodiments, the acidified mushroom filtrate or extract is concentrated (130) to accelerate decarboxylation of ibotenic acid into muscimol. In embodiments, the concentrating step comprises heating the acidified mushroom extract. In embodiments, the duration of heat exposure is from about 0.5 hours to about 6 hours. In embodiments, the extract is heated from about 75° C. to about 177° C. (200° F.-350° F.). In embodiments, the acidified mushroom filtrate or extract is concentrated to accelerate decarboxylation of ibotenic acid into muscimol. In some embodiments, the concentrating step comprises heating the acidified mushroom extract. In embodiments, the duration of heat exposure is from about 0.5 hours to about 6 hours, wherein the range is inclusive. In embodiments, the duration of heat exposure is about 0.5 h, 1 h, 1.5 hours, 2 hours, 2.5 hours, 3 hours, 3.5 hours, 4 hours, 4.5 hours, 5 hours, 5.5 hours, or 6 hours. In embodiments, the heat exposure exceeds 6 hours. In embodiments, the extract is heated from about 75° C. to about 177° C., including about 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., and values in between, wherein each value is modified by the term “about.”

In some embodiments, the concentration step comprises application of pressure. In some embodiments, the applied pressure is between about 10 psi and about 25 psi, including at least about 10 psi, 11 psi, 12 psi, 13 psi, 14 psi, 15 psi, 16 psi, 17 psi, 18 psi, 19 psi, 20 psi, 21 psi, 22 psi, 23 psi, 24 psi, 25 psi, and values in between, as would be apparent to one of skill, wherein the values are modified by the term about. In some preferred embodiments, the applied pressure is 15 psi. As is known to one of skill in the art, the pressure described herein can be converted to different units, e.g., 1 psi=6.9 kPa=0.069 bar.

In some embodiments, the concentrating is performed with use of heat and pressure. In some embodiments, distillation assists the conversion of ibotenic acid to muscimol. In some embodiments, refluxing assists the conversion of ibotenic acid to muscimol. In embodiments, a combination of distillation and refluxing assists the conversion of ibotenic acid to muscimol. In embodiments, pressure cooking assists the conversion of ibotenic acid to muscimol.

In some embodiments, a condenser may be used, a non-limiting example of which is a Pyrex Graham condenser. As is known in the art, a Graham condenser includes a coolant-jacketed spiral coil running the length of the condenser serving as the vapor-condensate path. The Graham condenser may be attached to another flask, such as a round bottom flask, for example, for collection. The Graham condenser may be placed in a downward position for distillation, so that the acidic mixture enters the condenser at the bottom. The unused neck is stoppered with a plug. In some embodiments, steps are taken to avoid light exposure throughout the process, due to the potential for muscimol degradation. In one non-limiting example, aluminum foil may be wrapped around the round bottom flask and up the condenser part way to lower light intensity inside the round bottom flask during refluxing. However, any such method of reducing light penetration is acceptable, including completing the process in a dark room, setting the flask itself in an enclosure, using an opaque flask that reflects light, etc.

Regardless of the heating means, and whether or not the mixture is agitated, high heat is applied (e.g., placing a heating mantle on “high”) until distillation begins and then is adjusted so that the rate of distillate collection is about 1 drop per second, for example. In some embodiments, such “high” heat is between about 110° C., to about 130° C., including 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., and values in between, wherein each value may be modified by the term “about.” In one example, distillation is performed for about 0.5 hours to about 6 hours. In some embodiments, distillation is performed for about 5 to about 6 hours. In another example, it is performed for about 2 to about 3 hours. In another example, it is performed for about 3 hours. Distillation for 3 hours, for example, has been found to increase the muscimol content by a significant amount and to modulate the amount of muscimol relative to ibotenic acid, as demonstrated in Example 1.

Another exemplary extraction procedure comprises the steps of: (a) performing an aqueous extraction of A. muscaria mushroom biomass in the presence of heat to produce an extract; (b) filtering the extract; (c) reducing the pH of the extract; and (d) heating the extract. In embodiments, the procedure further comprises the step of (e) increasing the pH of the extract.

In some embodiments, performing the aqueous extraction comprises mixing water with ground mushrooms, which may be dried and otherwise prepared for extraction as described in embodiments herein. In some embodiments, the temperature of the mixture of water and ground mushrooms is maintained at a temperature of about 70° C. to about 100° C., including about 70° C., 75° C., 80° C., 85° C., 90° C., 95° C., or 100° C. In some embodiments, the mixture is stirred for between about 5 minutes to about 20 minutes, including 5 minutes, 10 minutes, 15 minutes, 20 minutes, or greater than 20 minutes; at between 700 rpm and 2500 rpm, including about 750 rpm, 800 rpm, 850 rpm, 900 rpm, 950 rpm, 1000 rpm, 1050 rpm, 1100 rpm, 1150 rpm, 1200 rpm, 1250 rpm, 1300 rpm, 1350 rpm, 1400 rpm, 1450 rpm, 1500 rpm, 1550 rpm, 1600 rpm, 1650 rpm, 1700 rpm, 1750 rpm, 1800 rpm, 1850 rpm, 1900 rpm, 1950 rpm, 2000 rpm, 2050 rpm, 2100 rpm, 2150 rpm, 2200 rpm, 2250 rpm, 2300 rpm, 2350 rpm, 2400 rpm, 2450 rpm, and 2500 rpm. In some embodiments, the water:mushroom ratio is about 5:1, 10:1, 11:1, 12:1, 13:1, 14:1, 15:1, 16:1, 17:1, 18:1, 19:1 or 20:1, or values in between these ratios.

In some embodiments, the extract is then filtered to remove solids, such as particles of ground mushroom. Any filter known to those of skill may be utilized, so long as the filter is capable of filtering out both large and small particles. In fact, in some embodiments, a plurality of filters may be used in a sequential arrangement, wherein the filter pore size decreases with each additional filter. In such embodiments, at least two, at least three, at least four, at least five, or more than five filters may be utilized in series. This is particularly useful if certain particles of a given size are desired in the extract, or to be individually isolated. In some embodiments, pressure is applied during the filtration process, such as with use of mechanical pressing, compressed gas, or any suitable means of applying pressure to the feed side of a filtration apparatus. For example, in some embodiments, the extract may be drawn through the filter(s) by vacuum filtration. In an optional embodiment, the filtered extract (i.e., the filtrate) is centrifuged to separate particulate matter and a supernatant.

In some embodiments, the pH of the extract is then reduced by the addition of a suitable acid. Without being bound by theory, the acidic environment causes ibotenic acid to convert into muscimol by a decarboxylation reaction, as discussed above. In some embodiments, the pH is reduced from the initial pH of the extract, which may be about 7 (the pH of distilled water) to a pH of about 1.0 to about 4.0. In some embodiments, the pH of the extract is reduced to about 1.0, to about 1.5, to about 2.0, to about 2.5, to about 3.0, to about 3.5, or to about 4.0, wherein the range is inclusive. In some embodiments, the pH of the extract is reduced to between about 2.0 and 3.0. In some embodiments, the pH of the extract is reduced to between about 2.5 and 3.0. In some embodiments, the acid is a mineral acid—for example, hydrochloric acid (HCl), hydrofluoric acid (HF), hydrobromic acid (HBr), hydroiodic acid (HI), nitric acid, phosphoric acid, sulfuric acid, boric acid, or carbonic acid. In some embodiments, the acid is an organic acid—for example, acetic acid, citric acid, or oxalic acid. The acid can also be any other compound known to those of skill capable of lowering the pH of the extract.

In some embodiments, the extract is then heated. In some embodiments, the extract is heated to a temperature of between about 95° C. and about 105° C. In some embodiments, the extract is heated to a temperature of between about 95° C., 96° C., 97° C., 98° C., 99° C., 100° C., 101° C., 102° C., 103° C., 104° C., or 105° C. In some embodiments, the extract is heated to reflux. In some embodiments, the duration of the heating is about 1 to 6 hours. For example, the duration of the heating may be about 1 hour, 2 hours, 3 hours, 4 hours, 5 hours, or 6 hours, or times in between these values.

In some embodiments, the pH of the extract is then increased by addition of a suitable base. In some embodiments, the pH is raised to a pH of about 4 to about 11. For example, in some embodiments, the pH of the extract is raised to a pH of about 4, 4.5, 5, 6, 7, 8, 9, 10, 11, or PH levels in between these values. In some embodiments, the base is a hydroxide base (e.g., NaOH, KOH, Mg(OH) 2, Ca(OH) 2). In some embodiments, the base is a carbonate base (e.g., Na₂CO₃, K₂CO₃, MgCO₃, CaCO₃). In some embodiments, the base is a bicarbonate base (e.g., NaHCO₃, KHCO₃, NH₄HCO₃). In some embodiments, the base is added in solid form (e.g., as pellets, granules, or powder). In other embodiments, the base is added as a solution, such as an aqueous solution (e.g., aqueous NaOH). In preferred embodiments, the base is 1 M aqueous NaOH. However, the base can alternatively be any other compound known to those of skill capable of raising the pH of the extract.

### Example 1: Amanita Muscaria Extraction

Amanita muscaria mushrooms were obtained from Vashon Island, Washington. The mushrooms were harvested in the fall, dried to a moisture content of from about 2% to about 4% by weight, sealed in plastic 4-liter bags, and stored frozen in darkness at −15° C. until used. The mushrooms were allowed to thaw to room temperature, were then removed from the plastic bags, and were then inspected to ensure dryness so they can be easily ground to a powder. Dryness was determined by attempting to snap mushroom caps in half. Mushroom caps in the desired range of dryness snapped in half. This was determined one hour prior to beginning the extraction process.

Mushrooms that were not sufficiently dry were dehydrated in a forced air food dehydrator (internal dimensions, 30 cm×30 cm×30 cm) at 50° C. for 90 minutes. Drying continued until each mushroom was sufficiently dehydrated (about 2-3% moisture content by weight).

The dried mushrooms were mixed with distilled water. The pH of the distilled water was first measured after calibration with a pH meter. The pH of the distilled water was about 7. Since distilled water was stored in a plastic container, the distilled water was boiled to remove any organic material that may have leached into the water from the plastic container. 1000 mL of the distilled water was placed in a 2000 mL Pyrex beaker and boiled at 100° C. for 10 minutes in a microwave oven.

The mushroom caps were ground by a coffee grinder. Caps were ground for 45 seconds.

The beaker of boiled water was removed from the microwave oven and placed on a heating plate. Twenty-five grams of the ground mushroom caps were added to the hot water and gently stirred with a glass rod for 10 minutes. The temperature was maintained at 95° C. The ratio of mushroom caps to distilled water was 25 grams to 1000 ml water. The mixture of the ground mushroom caps in the heated water in the beaker was then filtered. A Buchner funnel was set up for vacuum filtration.

A 23-gram piece of 10 cm×10 cm×5 cm glass wool was cut in a circular shape approximately the same diameter as the Buchner funnel. It was placed on top of the glass filter in the funnel. Four grams of cheese cloth measuring 25 cm×30 cm was folded into four layers and placed on top of the glass wool. The cheese cloth captured larger particles while the glass wool captured smaller particles.

The filter setup was attached to a 1000 ml Erlenmeyer flask. The filter layers were seated by pouring 100 ml of the distilled water that was earlier boiled, through the setup with vacuum turned on. Filtrate was removed from the flask after pouring water through the filters.

To filter the mixture of ground mushroom and water, the mixture was allowed to settle for two minutes in the beaker. The mixture was then slowly decanted into the filter without the ground mushroom powder at the bottom of the beaker to pour into the filter until near the end of the procedure. This reduced clogging and maintained a better flow through the filter. The vacuum pump was turned on to begin to filter the extract, at a pressure of −0.7 bar.

When the extract was filtered, the filter set up as vacuum flushed with 30 ml of the distilled water that was previously boiled. The process yields from about 925 mL to about 975 ml of filtrate in the flask, when performed as a single run. The filtrate appeared cloudy.

The pH of the filtrate was adjusted from the pH of about 7 (the pH of the distilled water) to a pH of about 3.0 by the addition of 1 M hydrochloric acid. After addition of the hydrochloric acid, the filtrate was refluxed through a Pyrex Graham condenser having a coolant-jacketed spiral coil running the length of the condenser serving as the vapor-condensate path. The length of the Graham condenser was 43 cm. The length of the water jacket was 30 cm and it had 24/60 glass joints. The Graham condenser was attached to a 2000 ml round bottom flask for collection. The Graham condenser was in a downward position for distillation. The unused neck was stoppered with a glass 24/40 plug.

A round bottom flask was secured to the condenser with burette/test tube clamps that were attached to a generic 60 cm high ring stand. Aluminum foil was wrapped around the round bottom flask and up the condenser part way to lower light intensity inside the round bottom flask during refluxing. The Erlenmeyer flask was attached to the inlet of the Graham condenser.

The condenser coil was cooled with cold water pumped in by a pump. The setup Graham condenser was heated with a heating mantle/magnetic stirrer. The distillation was performed in a fume hood. Distillation took place at 250° F. (~121° C.) at 15 psi (~1.0 bar). A 2.5 cm magnetic stir bar was placed in the pH distillate to be distilled before starting distillation. During distillation, the magnetic stirring was provided at from about 30 rpm to about 60 rpm, to prevent bumping. The heating level was set on high until the distillation began and was then adjusted so that 1 drop per second was observed and collected.

The refluxed extract was then distilled for a period of 3 hours. Samples were taken prior to distillation, after 1 hour, 2 hours, and at 3 hours of distillation, as discussed further below. After distillation, the filtrate was cooled to 3° C. in a refrigerator. After about 3 hours of cooling, there was coagulated material suspended in the filtrate. The coagulated material in the filtrate was removed via vacuum filtration through the vacuum filtration setup described above (without the cheesecloth). After filtration, the filter setup was rinsed under vacuum with 30 mL of distilled water into the filtrate.

HPLC-MS/MS was used to analyze mushrooms and extracts at various stages of the process. The starting material for the extraction process had the following concentrations: muscimol (625 μg/g), muscarine (340 μg/g), and ibotenic acid (2930 μg/g).

HPLC-MS/MS analysis was completed at hourly timepoints of the 3 hour distillation. Prior to distillation, the extract contained 44.3 μg/g muscimol, 12.4 μg/g muscarine, and 228 μg/g ibotenic acid. One hour after distillation, the amount of muscimol increased from 44.3 μg/g to 144 μg/g, while the amount of ibotenic acid decreased from 228 μg/g to 44.9 μg/g. After two hours of distillation, muscimol levels increased to 162 μg/g, ibotenic acid decreased to 7.37 μg/g, and muscarine levels were 12.7 μg/g. After 3 hours of distillation, levels of muscimol decreased from 162 μg/g to 146 μg/g, levels of muscarine were measured at 12.1 μg/g, and ibotenic acid levels decreased to 1.38 μg/g. Thus, extracts with different relative amounts of muscimol, ibotenic acid, and muscarine (including, e.g., the weight ratio of muscimol to ibotenic acid) can be produced by altering the distillation time.

Quantitative analyses of the cadmium, arsenic, mercury, and lead content of the mushrooms were also performed by inductively coupled plasma mass spectrometry (“ICP-MS”). The cadmium content of the heated, ground mushroom powder was 10.2 parts per million (ppm), the arsenic content was 1.67 ppm, the mercury content was 1.18 ppm, and the lead content was 0.865 ppm. Prior to the start of reflux, the cadmium content was 0.073 ppm, the arsenic content was 0.018 ppm, the mercury content was below detection limits (less than 0.001 PPM), and the lead content was 0.011 ppm.

### Example 2: Amanita Muscaria Ground Powder Extract

In this example, a ground powder extract is provided. The same mushrooms as in Example 1 were pressed through a filter to obtain filtered extract. A total amount of 1.5 kg of mushrooms were inspected and dehydrated, and then cut into pieces having dimensions of about 1 cm×1 cm. The pH of distilled water was confirmed to be about 7 by a calibrated PH meter and the water was boiled for 10 minutes. The cut pieces of the mushrooms were placed in the boiled water. The temperature of the water was maintained at 95° C.

A fruit press was used to press the mushrooms through a filter to extract filtrate. A 1 L beaker was added to the bottom of the fruit press to collect the filtrate. Twenty-five grams of the heated mushroom pieces were added to a 125-micron filter bag having a volume of 40 liters.

The filter bag containing mushroom pieces was placed in the fruit press, and the pressure of the fruit press was increased in about 5-minute increments over the course of 1 hour to press the mushroom pieces through the filter bag. The process was repeated with a second bag containing 25 grams of heated mushroom pieces. 14-15 liters of filtrate were collected, having a moisture content of from about 50% to about 70%. Following filtration via the fruit press, the pH of the filtrate was reduced to a pH of about 3.0 by the addition of 1 M hydrochloric acid.

The filtrate was placed in a Pyrex beaker, placed on a heating plate in a microwave oven, and heated at 100% power for 2 hours to reduce the volume to 10% of the original amount, as measured with the Pyrex beaker.

After heating, the filtrate on visual inspection was thick and colored black, and it was therefore determined that the filtrate needed to be further dried or cured. The filtrate was heated again in the microwave oven at 80% power at one-minute intervals, with stirring in between, for a total of one hour, until the color of the filtrate turned from black to light brown, demonstrating the desired change in moisture content. The resulting filtrate had a thick appearance on visual inspection and was similar in physical consistency to bread dough upon tactile inspection.

As the filtrate was cooling, filtrate was cut into pieces having a length and width of from about 3 cm to about 4 cm. Each piece was ground into a fine powder using a coffee grinder. The ground power extract was placed into a sealed plastic bag, and stored in the dark at −15° C.

Testing specifications, certificates of analysis (COAs), and quantitative analyses for AME-1 extracts produced according to disclosed methods, such as in Examples 1 and 2, are shown in Tables 1-7 below.

TABLE 1
Testing Specifications for Exemplary A. muscaria Extract AME-1.

| | | |
|---|---|---|
| Parameter | Specification | Method |

| Color | Brown | | Organoleptic |
|---|---|---|---|
| Taste | Similar to meat gravy | | Organoleptic |
| Smell | Forest mushroom scent | | Organoleptic |
| Muscimol | <30000 | μg/g | HPLC-MS/MS |
| Muscarine | <550 | μg/g | HPLC-MS/MS |
| Ibotenic acid | <20 | μg/g | HPLC-MS/MS |
| Stizolobinic | <40 | μg/g | HPLC-MS/MS |
| acid | | | |
| Arsenic | <0.03 | ppm | ICP-MS/EPA 3052 |
| Cadmium | <0.09 | ppm | ICP-MS/EPA 3052 |
| Mercury | <0.02 | ppm | ICP-MS/EPA 3052 |
| Lead | <0.09 | ppm | ICP-MS/EPA 3052 |

| | | |
|---|---|---|
| Pesticides | Conforms | Conforms |

TABLE 2
COA of exemplary A. muscaria extract
Extract Amount: 20 g

| Description | | | | Pass |
|---|---|---|---|---|
| Assay | Method | Result | | or Fail |
| Muscimol | Non-compendial | 146 | μg/g | Pass |
| | in-house method | | | |
| | by HPLC-MS/MS | | | |
| Muscarine | Non-compendial | 12.1 | μg/g | Pass |
| | in-house method | | | |
| | by HPLC-MS/MS | | | |
| Ibotenic | Non-compendial | 1.38 | μg/g | Pass |
| acid | in-house method | | | |
| | by HPLC-MS/MS | | | |
| Cadmium | EPA 3052 | 0.073 | μg/g | Pass |
| Arsenic | EPA 3052 | 0.018 | μg/g | Pass |
| Mercury | EPA 3052 | ND | μg/g | Pass |
| Lead | EPA 3052 | 0.011 | μg/g | Pass |

TABLE 3
COA of exemplary A. muscaria extract
Extract Amount: 4300 g (solid)

| Description | | | | Pass |
|---|---|---|---|---|
| Assay | Method | Result | | or Fail |
| Muscimol | Non-compendial | 28356 | μg/g | Pass |
| | in-house method | | | |
| | by HPLC-MS/MS | | | |
| Muscarine | Non-compendial | 499.453 | μg/g | Pass |
| | in-house method | | | |
| | by HPLC-MS/MS | | | |
| Ibotenic acid | Non-compendial | 14.953 | μg/g | Pass |
| | in-house method | | | |
| | by HPLC-MS/MS | | | |
| Cadmium | EPA 3052 | 0.20 | μg/g | Pass |
| Arsenic | EPA 3052 | ND | μg/g | Pass |
| Mercury | EPA 3052 | 0.14 | μg/g | Pass |
| Lead | EPA 3052 | 0.019 | μg/g | Pass |
| Stizolobinic | | 0.025 | μg/g | Pass |
| acid | | | | |

TABLE 4
COA of exemplary A. muscaria extract

| Product Name: | Amanita muscaria | Product Lot | | 0001 |
|---|---|---|---|---|
| | Extract (AME-1) | Number: | | |
| Report Date: | Dec. 7, 2020 | Laboratory Number: | | 7809405373 |
| Extract Amount: | 120 g | Batch Release Date: | | Dec. 7, 2020 |
| Description | Method | Result | | Pass |
| Assay | | | | or Fail |
| Muscimol | Non-compendial | 16922 | μg/g | Pass |
| | in-house method | | | |
| | by HPLC-MS/MS | | | |
| Muscarine | Non-compendial | 1468 | μg/g | Pass |
| | in-house method | | | |
| | by HPLC-MS/MS | | | |
| Ibotenic | Non-compendial | 159 | μg/g | Pass |
| Acid | in-house method | | | |
| | by HPLC-MS/MS | | | |
| Cadmium | EPA 3052 | 0.14 | μg/g | Pass |
| Arsenic | EPA 3052 | 2.5 | μg/g | Pass |
| Mercury | EPA 3052 | 0.12 | μg/g | Pass |
| Lead | EPA 3052 | 1.6 | μg/g | Pass |

TABLE 5
Quantitative analysis of A. muscaria extract
meeting specs of exemplary extract AME-1

| | |
|---|---|
| Test Item Name: | Amanita muscaria Extract (AME-1) |
| Purity/Assay: | 1.69% muscimol, 0.15% muscarine, |
| | 0.02% ibotenic acid (potency) |
| Batch produced by: | Psyched Wellness |
| Color: | Tan |
| Physical Characteristics: | Powder, pH-3.0, water soluble |
| Quantity sent: | 120 g |
| Storage condition: | Room temperature (22 °C), long |
| | term storage (−20 °C) |

TABLE 6
COA of exemplary A. muscaria extract
Extract Amount: 80 L

| Description | | | | Pass |
|---|---|---|---|---|
| Assay | Method | Result | | or Fail |
| Muscimol | Non-compendial | 91.30 | μg/g | Pass |
| | in-house method | | | |
| | by HPLC-MS/MS | | | |
| Muscarine | Non-compendial | 8.48 | μg/g | Pass |
| | in-house method | | | |
| | by HPLC-MS/MS | | | |
| Ibotenic | Non-compendial | 8.04 | μg/g | Pass |
| acid | in-house method | | | |
| | by HPLC-MS/MS | | | |
| Cadmium | EPA 3052 | 0.20 | μg/g | Pass |
| Arsenic | EPA 3052 | 0.024 | μg/g | Pass |
| Mercury | EPA 3052 | 0.029 | μg/g | Pass |
| Lead | EPA 3052 | 0.021 | μg/g | Pass |
| Stizolobinic | Non-compendial | 19.52 | μg/g | Pass |
| acid | in-house method | | | |
| | by HPLC-MS/MS | | | |

TABLE 7
COA of exemplary A. muscaria extract
Extract Amount: 80 L

| Description | | | | Pass |
|---|---|---|---|---|
| Assay | Method | Result | | or Fail |
| Muscimol | Non-compendial | 105.0 | μg/g | Pass |
| | in-house method | | | |
| | by HPLC-MS/MS | | | |
| Muscarine | Non-compendial | 9.89 | μg/g | Pass |
| | in-house method | | | |
| | by HPLC-MS/MS | | | |
| Ibotenic acid | Non-compendial | 8.57 | μg/g | Pass |
| | in-house method | | | |
| | by HPLC-MS/MS | | | |
| Cadmium | EPA 3052 | 0.24 | μg/g | Pass |
| Arsenic | EPA 3052 | 0.020 | μg/g | Pass |
| Mercury | EPA 3052 | 0.034 | μg/g | Pass |
| Lead | EPA 3052 | 0.025 | μg/g | Pass |
| Stizolobinic | Non-compendial | 18.90 | μg/g | Pass |
| acid | in-house method | | | |
| | by HPLC-MS/MS | | | |

### D. Vaporizer Formulations

In some aspects are provided vaporizer formulations (also referred to herein as vaporizable formulations) comprising fungal material (e.g., Amanita muscaria mushroom biomass) or an extract or compound thereof, a base liquid, and optionally a flavorant. Vaporizer formulations may include such formulations as are referred to by such terms as are used and will be readily recognized in the art, including as non-limiting examples vape juice, e-liquid, e-juice, and the like, which may be used interchangeably herein, unless a specific type is indicated by context, and all of which will be understood to be within the scope and spirit of the disclosed invention.

Also provided are vaporizable formulations comprising an Amanita muscaria extract. In some embodiments, a disclosed vaporizable formulation comprises an A. muscaria extract and a base liquid. In some embodiments, a disclosed vaporizable formulation comprises an A. muscaria extract; a base liquid; and, optionally, a flavorant. Vaporizer formulations may be a liquid (including, e.g., an oil) and/or solid (including, e.g., a wax) solution.

In embodiments, the formulation comprises fungal material or an extract thereof in an amount of between 1% v/v, to 50% v/v of the formulation, wherein the range is inclusive. In embodiments, the formulation comprises one or more compounds thereof in an amount of between 0.01 w/v % to 20 w/v %.

In embodiments, the vaporizer formulation comprises fungal material, which may be Amanita muscaria mushrooms, such as dried and ground and/or pulverized A. muscaria mushrooms. That is, A. muscaria fungal material that has not been extracted, and, e.g., is used directly in the formulation.

In some embodiments, the vaporizer formulation may include one or more of fungal material, an extract thereof, and a compound thereof, including one or more of each of a fungal material, an extract thereof, and a compound thereof, i.e., one or more fungal materials, and/or one or more extracts thereof, and/or one or more compounds thereof. In embodiments, the fungal material(s), extract(s) thereof, and/or compound(s) thereof comprise the “fungal portion” of a vaporizer formulation.

In embodiments, the vaporizer formulation comprises a fungal extract. In embodiments, the fungal extract is an Amanita muscaria extract. In embodiments, the A. muscaria extract is an AME-1 extract. In embodiments, the A. muscaria extract is obtained as a liquid distillate. In embodiments, the Amanita muscaria extract is obtained as a ground powder.

In some embodiments, the Amanita muscaria extract comprises no potentially toxic compounds in amounts that are significant for human health. In embodiments, the A. muscaria extract comprises no stizolobinic acid and no more than 0.09 ppm of cadmium, 0.03 ppm of arsenic, 0.09 ppm of lead and 0.02 ppm of mercury. In embodiments, the A. muscaria extract comprises less than about 40 μg/g of stizolobinic acid. In embodiments, the A. muscaria extract comprises less than about 30 μg/g of stizolobinic acid. In embodiments, the A. muscaria extract comprises less than about 20 μg/g of stizolobinic acid. In embodiments, the A. muscaria extract comprises less than about 10 μg/g of stizolobinic acid. In embodiments, the A. muscaria extract comprises less than about 5 μg/g of stizolobinic acid. In embodiments, the A. muscaria extract comprises less than about 2 μg/g of stizolobinic acid. In embodiments, the A. muscaria extract comprises less than about 1 μg/g of stizolobinic acid. In embodiments, the A. muscaria extract comprises less than about 0.5 μg/g of stizolobinic acid. In embodiments, the A. muscaria extract comprises less than about 0.1 μg/g of stizolobinic acid. In embodiments, the A. muscaria extract comprises less than about 0.05 μg/g of stizolobinic acid. In embodiments, the A. muscaria extract comprises less than about 0.025 μg/g of stizolobinic acid. In embodiments, the A. muscaria extract does not contain an amount of stizolobinic acid that is detectable by HPLC-MS/MS.

In some embodiments, the Amanita muscaria extract comprises less than about 2.5 ppm of arsenic. In some embodiments, the Amanita muscaria extract comprises less than about 1 ppm of arsenic. In some embodiments, the Amanita muscaria extract comprises less than about 0.1 ppm of arsenic. In some embodiments, the Amanita muscaria extract comprises less than about 0.05 ppm of arsenic. In some embodiments, the Amanita muscaria extract comprises less than about 0.04 ppm of arsenic. In embodiments, the A. muscaria extract comprises less than about 0.03 ppm of arsenic. In embodiments, the A. muscaria extract comprises less than about 0.02 ppm of arsenic. In embodiments, the A. muscaria extract comprises less than about 0.018 ppm of arsenic. In embodiments, the A. muscaria extract comprises less than about 0.01 ppm of arsenic. In embodiments, the A. muscaria extract does not contain an amount of arsenic that is detectable by ICP-MS.

In some embodiments, the Amanita muscaria extract comprises less than about 0.30 ppm of cadmium. In some embodiments, the Amanita muscaria extract comprises less than about 0.25 ppm of cadmium. In some embodiments, the Amanita muscaria extract comprises less than about 0.20 ppm of cadmium. In some embodiments, the Amanita muscaria extract comprises less than about 0.10 ppm of cadmium. In embodiments, the A. muscaria extract comprises less than about 0.090 ppm of cadmium. In embodiments, the A. muscaria extract comprises less than about 0.080 ppm of cadmium. In embodiments, the A. muscaria extract comprises less than about 0.073 ppm of cadmium. In embodiments, the A. muscaria extract comprises less than about 0.070 ppm of cadmium. In embodiments, the A. muscaria extract does not contain an amount of cadmium that is detectable by ICP-MS.

In some embodiments, the Amanita muscaria extract comprises less than about 0.15 ppm of mercury. In some embodiments, the Amanita muscaria extract comprises less than about 0.10 ppm of mercury. In some embodiments, the Amanita muscaria extract comprises less than about 0.05 ppm of mercury. In embodiments, the A. muscaria extract comprises less than about 0.04 ppm of mercury. In embodiments, the A. muscaria extract comprises less than about 0.03 ppm of mercury. In embodiments, the A. muscaria extract comprises less than about 0.02 ppm of mercury. In embodiments, the A. muscaria extract comprises less than about 0.015 ppm of mercury. In embodiments, the A. muscaria extract comprises less than about 0.01 ppm of mercury. In embodiments, the A. muscaria extract does not contain an amount of mercury that is detectable by ICP-MS.

In some embodiments, the Amanita muscaria extract comprises less than about 2.5 ppm of lead. In some embodiments, the Amanita muscaria extract comprises less than about 1 ppm of lead. In some embodiments, the Amanita muscaria extract comprises less than about 2 ppm of lead. In some embodiments, the Amanita muscaria extract comprises less than about 1.6 ppm of lead. In some embodiments, the Amanita muscaria extract comprises less than about 1 ppm of lead. In some embodiments, the Amanita muscaria extract comprises less than about 0.1 ppm of lead. In some embodiments, the Amanita muscaria extract comprises less than about 0.10 ppm of lead. In embodiments, the A. muscaria extract comprises less than about 0.09 ppm of lead. In embodiments, the A. muscaria extract comprises less than about 0.08 ppm of lead. In embodiments, the A. muscaria extract comprises less than about 0.07 ppm of lead. In embodiments, the A. muscaria extract comprises less than about 0.06 ppm of lead. In embodiments, the A. muscaria extract comprises less than about 0.05 ppm of lead. In embodiments, the A. muscaria extract comprises less than about 0.04 ppm of lead. In embodiments, the A. muscaria extract comprises less than about 0.03 ppm of lead. In embodiments, the A. muscaria extract comprises less than about 0.02 ppm of lead. In embodiments, the A. muscaria extract comprises less than about 0.015 ppm of lead. In embodiments, the A. muscaria extract comprises less than about 0.011 ppm of lead. In embodiments, the A. muscaria extract comprises less than about 0.01 ppm of lead. In embodiments, the A. muscaria extract does not contain an amount of lead that is detectable by ICP-MS. In embodiments, the A. muscaria extract comprises less than or no more than the limits for respective pesticides in USP General Chapter 561, “Articles of Botanical Origin” (USP 561). In embodiments, the extract is also in compliance with EPA (40 C.F.R. § 180) and FDA action levels (21 C.F.R. §§ 109, 509). In embodiments, the extract is manufactured in compliance with GLP or GMP requirements.

One of skill may use known methods to determine the presence of contaminants, one such method being HPLC-MS/MS. In some embodiments, HPLC-MS/MS may be used to determine the presence and concentration of other target compounds in the Amanita muscaria extract. In embodiments, HPLC-MS/MS is used to determine the concentration of muscimol, ibotenic acid, and muscarine.

In some embodiments, the Amanita muscaria extract comprises muscimol and ibotenic acid in a ratio of at least 3:1, where the ratio is preferably a mass (i.e., weight) ratio. In embodiments, the ratio also may be a mole ratio. In embodiments, the muscimol to ibotenic acid ratio is from about 3:1 to about 150:1. In embodiments, the muscimol to ibotenic acid ratio is about 10:1, 12.5:1 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, or 150:1, including ranges in between these values. In embodiments, the muscimol to ibotenic acid ratio is between about 10:1 and about 50:1. In embodiments, the muscimol to ibotenic acid ratio is between about 10:1 and about 40:1. In embodiments, the muscimol to ibotenic acid ratio is between about 10:1 and about 30:1. In embodiments, the muscimol to ibotenic acid ratio is between about 10:1 and about 20:1. In embodiments, the muscimol to ibotenic acid ratio is between about 10:1 and about 15:1. In embodiments, the muscimol to ibotenic acid ratio is between about 15:1 and about 50:1. In embodiments, the muscimol to ibotenic acid ratio is between about 15:1 and about 30:1. In embodiments, the muscimol to ibotenic acid ratio is between about 20:1 and about 50:1. In embodiments, the muscimol to ibotenic acid ratio is between about 20:1 and about 30:1. In embodiments, the Amanita muscaria extract comprises muscimol and ibotenic acid in a mass ratio of between about 20:1 and about 150:1. In embodiments, the A. muscaria extract comprises muscimol and ibotenic acid in a mass ratio of greater than about 100:1. In embodiments, the A. muscaria extract comprises muscimol and ibotenic acid in a mass ratio of greater than about 1000:1. In embodiments, the A. muscaria extract comprises muscimol and ibotenic acid in a mass ratio of about 12.5:1. In embodiments, the A. muscaria extract comprises muscimol and ibotenic acid in a mass ratio of about 25:1.

In embodiments, the Amanita muscaria extract comprises between about 0.5% and about 5.0% w/w or w/v muscimol, between about 0.05% and about 0.5% w/w or w/v muscarine, and less than about 0.05% w/w or w/v ibotenic acid.

In some embodiments, the Amanita muscaria extract comprises less than 18 mg/g of muscimol, less than 600 μg/g of muscarine, and less than 20 μg/g of ibotenic acid. In embodiments, the A. muscaria extract comprises less than about 550 μg/g of muscarine. In embodiments, the A. muscaria extract comprises less than about 500 μg/g of muscarine. In embodiments, the A. muscaria extract comprises less than about 250 μg/g of muscarine. In embodiments, the A. muscaria extract comprises less than about 200 μg/g of muscarine. In embodiments, the A. muscaria extract comprises less than about 100 μg/g of muscarine. In embodiments, the A. muscaria extract comprises less than about 50 μg/g of muscarine. In embodiments, the A. muscaria extract comprises less than about 25 μg/g of muscarine. In embodiments, the A. muscaria extract comprises less than about 15 μg/g of muscarine.

In embodiments, the Amanita muscaria extract comprises at least 15,000 μg/g. In embodiments, as illustrated in Table 4, the A. muscaria extract comprises between about 500 μg/g and 20,000 μg/g, wherein the range is inclusive.

In embodiments, the Amanita muscaria extract comprises less than 250 μg/g of ibotenic acid. In embodiments, as illustrated in Table 4, the Amanita muscaria extract comprises between about 240 μg/g and about 50 μg/g.

In embodiments, the potency of muscimol in the Amanita muscaria extract is at least about 0.25%, including about 0.25%, 0.5%, 0.75%, 1%, 1.25%, 1.5%, 1.75%, 2.0%, and greater than 2%, as may be obtained through utilizing the methods of the invention as disclosed herein. In embodiments, potency is equivalent to concentration.

In embodiments, the Amanita muscaria extract comprises ibotenic acid having a potency of less than 0.025%. In embodiments, potency can be determined by w/w %, e.g., by dividing the weight of a compound to be assessed with the total weight of an extract. For example, in an exemplary embodiment, and using the Certificate of Analysis of Table 4, which shows the weight of compounds of Product Lot Number 0001, the potency of muscimol is determined by dividing the weight of muscimol (16922 μg, i.e., 0.016922 g) per equivalent weight of the extract (1 g) to obtain a potency of 0.016922/1 or 1.69% muscimol, as also shown in Table 5.

In embodiments, the Amanita muscaria extract has a muscimol purity of at least 90%, including about 90%, 91%, 92%, 93%, 94%, 95%, 96%, 97%, 98%, 99%, and greater than 99%. In embodiments, the purity of a compound can be determined by dividing the weight of the compound by the total weight of the compounds against which its purity is measured (i.e., as a w/w %). For example, in an exemplary embodiment, and using the Certificate of Analysis of Table 4, which shows the weight of compounds of Product Lot Number 0001, the purity of muscimol is determined by dividing the weight of muscimol (16922 μg) by the total weight of the muscimol and the other compounds, i.e., muscarine (1468 μg), ibotenic acid (159 μg/g), and the heavy metals cadmium (0.14 μg), arsenic (2.5 μg), mercury (0.12 μg), and lead (1.6 μg), which in total equal 18553.36 μg, in other words, to divide 16922 μg by 18553.36 μg, which results in a purity of 91.21% muscimol. In embodiments, a purity of muscimol may be determined by dividing the weight of muscimol (16922 μg) by the total weight of the muscimol and the other compounds (i.e., muscarine and ibotenic acid), but not any heavy metals (e.g., cadmium, arsenic, mercury, or lead). Here, such purity will be 16922/18549, which equals a purity of 91.23% muscimol.

In some embodiments, the Amanita muscaria extract, which may optionally be further concentrated, is standardized. In embodiments, the Amanita muscaria extract is standardized to a muscimol purity of 90% or greater. In embodiments, the Amanita muscaria extract is standardized to a muscimol potency of between about 0.5% and about 5.0%.

A “standardized” extract refers to an extract comprising a specified quantity of a standardized ingredient, which may be a bioactive compound such as muscimol. Thus, in embodiments, an amount of the bioactive compound, such as an amount of muscimol, is standardized to a particular concentration (e.g., w/w or w/v % of the extract).

In some embodiments, an Amanita muscaria extract will be standardized so as to contain by weight percent an amount of muscimol (i.e., mg muscimol per mg or mL of extract, depending on whether such extract is a dry powder or a liquid) of between 0.5% and 5.0% w/w or w/v muscimol, wherein the range is inclusive.

Other standardizations w/w or w/v muscimol may be used in various embodiments, for example in amounts of between 5.0% and 10.0% or greater than 10% muscimol and also including amounts lower than those explicitly listed above.

In some embodiments, the Amanita muscaria extract will contain by weight percent an amount of muscarine (i.e., mg muscarine per mg extract) of at least 0.05% w/w or w/v muscarine.

Standardization may be accomplished by various methods, such as by measuring a concentration of compound in an extract to be standardized, determining the concentration of the compound to be standardized, determining an amount of excipient necessary to obtain a desired (standardized) concentration, and then adding the amount of excipient necessary to obtain the desired (standardized) concentration, resulting in a standardized extract. An excipient will be as known by ordinary skill, and may be a dry or liquid excipient, to create a dry powder or liquid standardized extract. Optionally, the concentration of standardized compound in standardized extract may be measured after adding one or more portions of excipient or after the final standardized extract is prepared, to confirm the standardization method and for quality control.

In some embodiments, the Amanita muscaria extract is further concentrated so that the bioactive compounds (including, and in particular muscimol) are increased in total concentration from an initial extract, such as an increase in w/w % (for a powder extract) or w/v % (for a liquid extract), in an amount such as by at least 5%, at least 10%, at least 15%, at least 20%, at least 25%, at least 30%, at least 40%, at least 45%, at least 50%, at least 60%, at least 70%, at least 80%, at least 90%, at least 100% (2×), at least 125%, at least 150%, at least 175%, at least 200% (3×), at least 250%, at least 300% (4×), at least 400% (5×), at least 500% (6×), at least 600% (7×), at least 700% (8×), at least 800% (9×), at least 900% (10×), and in amounts of 1,000% or more. Various methods of concentrating an extract will be known by reference to this disclosure. In one exemplary embodiment, an extract will be concentrated by evaporating a portion or an entirety of a solvent to create a volume of concentrated slurry at a desired concentration.

In embodiments, the vaporizer formulation comprises a compound from fungal material, which may be Amanita muscaria mushrooms, such as either or both of muscimol and muscarine.

In some embodiments, the one or more compounds includes ibotenic acid; however, it will be understood that In embodiments the concentration of ibotenic acid is relatively low (e.g., in relation to unextracted Amanita muscaria fungal material), trace, or zero.

In embodiments, the formulation comprises muscimol having a potency of at least 0.15% w/v.

In embodiments, the formulation comprises ibotenic acid having a potency of less than 0.0025% w/v.

In embodiments, the formulation is standardized to a muscimol potency of between about 0.01% w/v and about 2% w/v.

In embodiments, the formulation comprises between about 0.1 mg/ml and about 200 mg/ml of muscimol, inclusive. In embodiments, the formulation comprises between about 1 mg/ml and about 20 mg/ml of muscimol, inclusive. In embodiments, the formulation comprises at least 15 mg/ml of muscimol.

In embodiments, the formulation comprises between about 0.1 mg/mL and about 200 mg/ml of muscarine, inclusive. In embodiments, the formulation comprises between about 1 mg/ml and about 20 mg/ml of muscarine, inclusive.

In embodiments, the formulation comprises less than about 2 mg/ml of ibotenic acid, inclusive. In embodiments, the formulation comprises less than about 0.2 mg/ml of ibotenic acid, inclusive.

In some embodiments, the formulation comprises less than 20 mg/ml of muscimol, less than 1 mg/ml of muscarine, and less than 0.02 mg/ml of ibotenic acid.

In some embodiments, the compounds from Amanita muscaria are provided in A. muscaria fungal material (i.e., the vaporizer formulation comprises A. muscaria fungal material, which comprises one or more compounds from A. muscaria).

In some embodiments, the compounds from Amanita muscaria are provided in an A. muscaria extract (i.e., the vaporizer formulation comprises an A. muscaria extract, which comprises one or more compounds from A. muscaria).

In some embodiments, the compounds from Amanita muscaria are provided directly, e.g., as pure or substantially pure chemical compounds that are isolated or synthesized (i.e., the vaporizer formulation comprises one or more compounds from A. muscaria). In some embodiments, the compounds are obtained commercially, e.g., from Cayman Chemical Company, Ann Arbor, Michigan (e.g., Muscimol, Item No. 13667; Muscarine, Item No. 24269).

In embodiments, the formulation comprises between 40% v/v to 90% v/v of base liquid, wherein the range is inclusive. Where the formulation comprises one or more compounds from fungi, such as Amanita muscaria, or from an extract thereof, the formulation may comprise up to about 99.99% v/v base liquid, e.g., where the compounds from A. muscaria material or extract are 0.01 w/v % of the formulation.

In embodiments, the base liquid comprises one or more of propylene glycol (PG) and vegetable glycerin (VG). In embodiments, the base liquid comprises between 1% v/v to 100% v/v of propylene glycol, wherein the range is inclusive. In embodiments, the base liquid comprises between 1% v/v to 100% v/v of vegetable glycerin, wherein the range is inclusive.

In embodiments, the base liquid may be a base liquid obtained in a specific proportion. Common proportions include 50:50 (PG/VG), 30:70 (PG/VG), and 20:80 (PG/VG). However, said proportions should not be construed as limiting, and the base liquid may comprise anywhere from 1% to 100% PG, and/or anywhere from 1% to 100% VG.

In embodiments, one may substitute PG and/or VG for polyethylene glycol (PEG), such that the base liquid comprises anywhere from 1% to 100% PEG, with the remaining base liquid, if applicable, comprising a proportion of PG and/or VG.

In embodiments, the base liquid may further comprise water, ethanol, or a mixture thereof. In embodiments, the water may be tap water, mineral water, spring water, glacier water, distilled water, purified water, deionized water, and/or alkaline water. In embodiments, the mixture of water and ethanol is between about 10% v/v to about 99% v/v ethanol, wherein the range is inclusive. In embodiments, the mixture of water and ethanol is an alcoholic drink or spirit, including vodka.

In some embodiments, the mass ratio of the Amanita muscaria extract to the base liquid is about 1 part extract to about 2 parts base liquid. In embodiments, the mass ratio of the A. muscaria extract to the base liquid is about 1 part extract to about 5 parts base liquid. In embodiments, the mass ratio of the A. muscaria extract to the base liquid is about 1 part extract to about 10 parts base liquid. In embodiments, the mass ratio of the A. muscaria extract to the base liquid is about 1 part extract to about 15 parts base liquid. In embodiments, the mass ratio of the A. muscaria extract to the base liquid is about 1 part extract to about 20 parts base liquid. In embodiments, the mass ratio of the A. muscaria extract to the base liquid is about 1 part extract to about 25 parts base liquid. In embodiments, the mass ratio of the A. muscaria extract to the base liquid is about 1 part extract to about 30 parts base liquid. In embodiments, the mass ratio of the A. muscaria extract to the base liquid is about 1 part extract to about 40 parts base liquid. In embodiments, the mass ratio of the A. muscaria extract to the base liquid is about 1 part extract to about 50 parts base liquid.

In some embodiments, disclosed vaporizable formulations comprise an additional excipient. In embodiments, suitable excipients to be incorporated in disclosed formulations may include a flavoring agent (i.e., a flavorant), a colorant, a dye, a pigment, an antioxidant, a solvent, a humectant, a viscosity modifier, a solubilizer, a complexing agent, a preservative, a pH adjusting agent, an opacifier, a surfactant, a gelling agent, or a combination thereof.

In some embodiments, disclosed formulations may include a flavoring agent or a mixture of flavoring agents including natural or synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils, essences, limonene, menthone, carvone, menthol, anethole, eucalyptol, anethole, eugenol, cassia, oxanone, α-irisone, propenyl guaiethol, thymol, linalool, benzaldehyde, cineole, cinnamaldehyde, cinnamaldehyde glycerol acetal (CGA), methone glycerol acetal (MGA), 3-I-menthoxypropane-1,2-diol, N-ethyl-p-menthan-3-carboxamine, N,2,3-trimethyl-2-isopropylbutanamide, and combinations thereof.

In some embodiments, a vaporizer formulation comprises fungal material or an extract or compound thereof, a base liquid, and optionally a flavorant. In embodiments, the formulation comprises between about 1% v/v and about 50% v/v of a flavorant, wherein the range is inclusive. In embodiments, the formulation comprises between about 1% v/v and about 20% v/v of a flavorant. In embodiments, the formulation comprises between about 1% v/v and about 10% v/v of a flavorant. In embodiments, the formulation comprises between about 0.1% v/v and about 20% v/v of a flavorant. In embodiments, the formulation comprises between about 0.1% v/v and about 10% v/v of a flavorant. In embodiments, the formulation comprises between about 0.1% v/v and about 5% v/v of a flavorant. In embodiments, the formulation comprises between about 0.1% v/v and about 1% v/v of a flavorant.

In some embodiments a formulation comprises a flavoring agent, which may be a flavor. A flavor may be any of a liquid flavor, a powder flavor, a powdered encapsulated flavor, a spray-dried flavor, an emulsion flavor, a plant, fruit, vegetable, or other extraction-based flavor, a seasoning, an organic flavor, a natural flavor, a non-GMP flavor, a clean label flavor, a concentrated flavor, a savory flavor, a sweet flavor, a masking flavor, and the like, including combinations thereof. Flavors may be produced using methods known to those in the art, or obtained from flavor suppliers as known in the art.

Flavorants include flavor concentrates known to those of skill, for example, flavor concentrates that replicate different food and drink flavors. In embodiments, exemplary flavors include peach, mango, pineapple, strawberry, kiwi, watermelon, lime, green apple, strawberry watermelon, apple, peach strawberry, mango peach guava, kiwi dragonberry, peach pear, blueberry, raspberry, lemon, Fiji melons, passionfruit orange guava, honeydew, banana, lychee, blueberry lemon, dragonfruit, apple, fruit cocktail, watermelon, key lime, cannoli, cookie butter, cinnamon funnel cake, sweet cinnamon banana, strawberry cheesecake, crème brulee, strawberry a la mode, lemon crumble cake, vanilla bean ice cream, glazed donut, coffee ice cream, birthday cannoli, banana cream pie, caramel brulee, a la mode, strawberry milkshake, lemon drop, pink lemonade, blackberry lemonade, caramel coffee, Caribbean punch, piña colada, java yogi, Caribbean rum, cherry lime cola, spearmint, bubblegum grape, butterscotch, bubblegum, bubblegum melon, vanilla, and cornbread. Compounds providing the above or other flavors or flavor profiles are commercially available, and methods of incorporating flavors into vaporizer formulations will be readily appreciated in view of this disclosure and the knowledge in the art.

Colorants and/or dyes and/or pigments may be added to disclosed formulations in some embodiments. Suitable colorants and/or dyes and/or pigments may include colors such as e.g., white, black, yellow, blue, green, pink, red, orange, violet, indigo, brown, and combinations thereof, pigments such as, e.g., Timica Extra Large Sparkles, titanium dioxide and chromium oxide greens, ultramarine blues and pinks and ferric oxides. Colorants and/or dyes and/or pigments may be present, individually or in total (if more than one colorant and/or dye and/or pigment is included), in disclosed formulations in an amount ranging from about 0.01 wt % to about 5 wt % (calculated as the total weight of colorants and/or dyes and/or pigments in the formulation divided by the total weight of the formulation). In embodiments, a disclosed formulation does not comprise a colorant. In embodiments, a disclosed formulation does not comprise a dye. In embodiments, a disclosed formulation does not comprise a pigment.

Antioxidants may be added to disclosed formulations in some embodiments. Suitable antioxidants may include, but not be limited to, natural antioxidants such as tocopherol and tocopherol acetate. Antioxidants may be present, individually or in total (if more than one antioxidant is included), in disclosed formulations in an amount ranging from about 0.1 wt % to about 5 wt % (calculated as the total weight of antioxidants in the formulation divided by the total weight of the formulation). In embodiments, a disclosed formulation does not comprise an antioxidant.

Solvents that may be included in disclosed formulations may include, without limitations, water, polyhydric alcohols (e.g., glycerin), 1,3-butylene glycol, propylene glycol, hexylene glycol, propane diol, ethylene glycol, diethylene glycol, dipropylene glycol, diglycerin, sorbitol, other sugars which are liquid at room temperature, water-soluble alkoxylated nonionic polymers such as polyethylene glycol, and combinations thereof. Solvents may be present, individually or in total (if more than one solvent is included), in the formulation in an amount ranging from about 0.1 wt % to about 95 wt % (calculated as the total weight of solvents in the formulation divided by the total weight of the formulation). In embodiments, a disclosed formulation does not comprise a solvent.

Humectant(s), such as low molecular weight polyethylene glycol (e.g., PEG6-PEG12), may be present, individually or in total (if more than one humectant is included), in the formulation in an amount of up to about 10 wt %, up to about 5 wt %, up to about 3 wt %, up to about 1 wt %, or up to about 0.1 wt % (calculated as the total weight of humectants in the formulation divided by the total weight of the formulation). In embodiments, a disclosed formulation does not comprise a humectant.

Surfactants may be incorporated in disclosed formulations. The surfactants that can be included in the formulation may be anionic, nonionic, or amphoteric compounds. Suitable examples of anionic surfactants are one or more of higher alkyl sulfates such as potassium or sodium lauryl sulfate, higher fatty acid monoglyceride monosulfates, such as the salt of the monosulfated monoglyceride of hydrogenated coconut oil fatty acids, alkyl sulfonates such as sodium dodecyl benzene sulfonate, higher fatty sulfoacetates, higher fatty acid esters of 1,2 dihydroxypropane sulfonate. Examples of water soluble nonionic surfactants are condensation products of ethylene oxide with various hydrogen-containing compounds that are reactive therewith and have long hydrophobic chains (e.g., aliphatic chains of about 12 of 20 carbon atoms), which condensation products contain hydrophilic polyoxyethylene moieties, such as condensation products of poly(ethylene oxide) with fatty acids, fatty alcohols, fatty amides and other fatty moieties, and with propylene oxide and polypropylene oxides, e.g., Pluronic materials such as Pluronic F127. Exemplary suitable alkyl polyglycoside (APG) surfactant(s) that may be used in the formulation may comprise APG C8-C10, APG C10-C16, decyl glucoside, coco-glucoside, anionic APG carboxylate, sodium lauryl glucose carboxylate, lauryl glucoside, D-glucopyranose (oligomeric, CIO-16 glycosides, carboxymethyl ethers, sodium salts), C12-C16 fatty alcohol glycoside, and combinations thereof. Exemplary APG surfactant(s) that may be used may have an industry designation of Plantaren® 2000 N UP/MB, Plantapon® LGC Sorb, Plantaren® 1200 N UP/MB, and Plantaren® 818 UP/MB. Surfactants may be present, individually or in total (if more than one surfactant is included) in the formulation in an amount ranging from about 0.01 wt % to about 10 wt % (calculated as the total weight of surfactants in the formulation divided by the total weight of the formulation). In embodiments, a disclosed formulation does not comprise a surfactant.

Exemplary gelling agent(s) that may be issued in disclosed formulations may comprise pectins, starches, and gelatin forms derived from animals or from plants (e.g., pork gelatin) or a different gelling agent that would be suitable for consumption by individuals who do not consume meat products (e.g., vegetarians, vegans). The pectin in the formulation may include, e.g., high methoxyl pectin, low methoxyl pectin, or a combination thereof. In embodiments, the pectin is amidated pectin. In other embodiments, the pectin is non-amidated pectin. In certain embodiments, the pectin is a combination of amidated pectin and non-amidated pectin. The gelatin in the formulation may include Type A gelatin, Type B gelatin, a hide or skin gelatin (e.g., calf skin, pig skin) and/or a bone gelatin (e.g., calf bone, pig bone) used alone or in combination. Gelling agent(s) may be present, individually or in total (if more than one gelling agent is included) in the formulation in an amount ranging from about 0.1 wt % to about 20 wt % (calculated as the total weight of gelling agents in the formulation divided by the total weight of the formulation). In embodiments, a disclosed formulation does not comprise a gelling agent.

In some embodiments, disclosed formulations may comprise a preservative. Preservatives can be used to inhibit microbial growth or increase stability of the formulation, thereby prolonging the shelf life of the formulation. Suitable preservatives are known in the art and include EDTA, EGTA, benzalkonium chloride or benzoic acid or benzoates (e.g., sodium benzoate), vitamin A, vitamin C (ascorbic acid), citric acid, vitamin E, and tocopherol. In embodiments, a disclosed formulation does not comprise a preservative.

Also provided are methods of producing (i.e., manufacturing) disclosed vaporizable formulations.

In one aspect, with reference to FIG. 1, provided is a method of producing a vaporizable formulation, comprising: (a) performing an aqueous extraction of Amanita muscaria mushroom biomass in the presence of heat to produce the extract (e.g., 115); (b) reducing the pH of the extract (e.g., 125); (c) concentrating the extract (e.g., 130); and (d) dissolving the extract in a base liquid to produce the vaporizable formulation (e.g., 135).

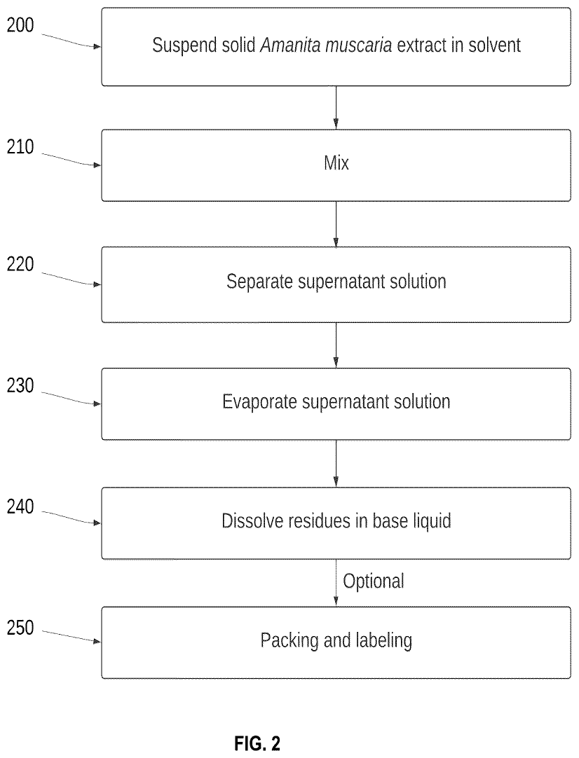

In another aspect, with reference to FIG. 2, provided is a method of producing a vaporizable formulation, comprising: (a) suspending an Amanita muscaria extract (e.g., a solid A. muscaria extract) in a solvent (e.g., 200); (b) mixing the suspension (e.g., 210); (c) isolating the supernatant from the suspension, thereby producing an extract solution (e.g., 220); (d) evaporating the extract solution to provide a residue (e.g., 230); and (e) dissolving the residue in a base liquid to produce the vaporizable formulation (e.g., 240).

In some embodiments, the Amanita muscaria extract is a solid A. muscaria extract. In embodiments, the A. muscaria extract is a ground powder extract (e.g., as described in Example 2). In embodiments, the A. muscaria extract is AME-1 in solid form.

In some embodiments, the Amanita muscaria extract is suspended in a solvent (200). The solvent can be any solvent suitable for extraction. For example, In embodiments, the solvent is an aqueous solvent (e.g., water, or a mixture that consists primarily of water). In other embodiments, the solvent is an organic solvent (e.g., an alcohol, ester, ether). In embodiments, the solvent is an alcohol (e.g., methanol, ethanol, propanol). In embodiments, the solvent is methanol. In embodiments, the A. muscaria extract is mixed with the solvent at a fixed ratio (e.g., a fixed ratio of the weight of the A. muscaria extract to the volume of solvent). In embodiments, the ratio of A. muscaria extract to solvent is from about 1:1 to about 1:100. In embodiments, the ratio of A. muscaria extract to solvent is from about 1:1 to about 1:50. In embodiments, the ratio of A. muscaria extract to solvent is from about 1:1 to about 1:20. In embodiments, the ratio of A. muscaria extract to solvent is from about 1:5 to about 1:20. In embodiments, the ratio of A. muscaria extract to solvent is from about 1:5 to about 1:15. In embodiments, the ratio of A. muscaria extract to solvent is from about 1:5 to about 1:10. In embodiments, the ratio of A. muscaria extract to solvent is from about 1:10 to about 1:20. In embodiments, the ratio of A. muscaria extract to solvent is from about 1:10 to about 1:15. In embodiments, the ratio of A. muscaria extract to solvent is about 1:10. In embodiments, the solvent is an alcohol, and the ratio of A. muscaria extract to the alcohol solvent is about 1:10. For example, In embodiments, the solvent is methanol, and the ratio of A. muscaria extract to methanol is about 1:10.

In some embodiments, the mixture is then mixed (210) for between about 1 minute to about 20 minutes, including 1 minute, 2, minutes, 5 minutes, 10 minutes, 15 minutes, 20 minutes, or greater than 20 minutes, including ranges in between these values. In embodiments, the mixture is mixed for about 15 minutes. In embodiments, wherein the mixture is stirred (e.g., using a magnetic stir plate), the stirring is performed at between 700 rpm and 2500 rpm, including about 750 rpm, 800 rpm, 850 rpm, 900 rpm, 950 rpm, 1000 rpm, 1050 rpm, 1100 rpm, 1150 rpm, 1200 rpm, 1250 rpm, 1300 rpm, 1350 rpm, 1400 rpm, 1450 rpm, 1500 rpm, 1550 rpm, 1600 rpm, 1650 rpm, 1700 rpm, 1750 rpm, 1800 rpm, 1850 rpm, 1900 rpm, 1950 rpm, 2000 rpm, 2050 rpm, 2100 rpm, 2150 rpm, 2200 rpm, 2250 rpm, 2300 rpm, 2350 rpm, 2400 rpm, 2450 rpm, and 2500 rpm, and ranges in between these values.

In some embodiments, the temperature of the mixture is approximately ambient temperature, or room temperature. In embodiments, the mixture is heated (e.g., by using a hot plate, water/oil bath, sand bath, or any other suitable means known to one of skill) to facilitate the extraction. In embodiments, the mixture is heated to a temperature of about 30° C. to about 100° C., including about 30° C., 40° C., 50° C., 60° C., 70° C., 80° C., 90° C., 95° C., or 100° C., and ranges in between these values. In embodiments, the mixture is not heated.

In some embodiments, the solvent (e.g., methanol) and the solid components of the supernatant (containing, e.g., residual Amanita muscaria extract or portions thereof) are separated (220). In embodiments, the mixture is filtered to remove solids. In some such embodiments, any filter known to those of skill may be utilized, so long as the filter is capable of filtering out both large and small particles. In fact, In embodiments, a plurality of filters may be used in a sequential arrangement, wherein the filter pore size decreases with each additional filter. In embodiments, the filter is a sock filter. In embodiments, the sock filter has a pore size of about 50 microns to about 500 microns. In embodiments, the sock filter has a pore size of about 50 microns, 75 microns, 100 microns, 150 microns, 200 microns, 250 microns, 300 microns, or 500 microns. In embodiments, a plurality of filters may be used in a parallel arrangement, wherein the filter pore size is the same for each filter. In such embodiments, at least two, at least three, at least four, at least five, or more than five filters may be utilized in parallel. In embodiments, pressure is applied during the filtration process, such as with use of mechanical pressing, compressed gas, or any suitable means of applying pressure to the feed side of a filtration apparatus.

In some embodiments, the solvent (e.g., methanol) and the solids are separated by centrifugation. In embodiments, the mixture is centrifuged for between about 1 minute to about 20 minutes, including 1 minute, 2, minutes, 5 minutes, 10 minutes, 15 minutes, 20 minutes, or greater than 20 minutes, including ranges in between these values. In embodiments, the mixture is centrifuged for 5 minutes. In embodiments, the mixture is centrifuged at between about 5,000 rpm and 30,000 rpm, including about 5,000 rpm, about 10,000 rpm, about 15,000 rpm, about 20,000 rpm, about 25,000 rpm, and about 30,000 rpm, including ranges in between these values. In embodiments, the mixture is centrifuged at about 15,000 rpm. For example, the mixture may be centrifuged at about 15,000 rpm for about 5 minutes.

In some embodiments, isolation of the supernatant solution from the mixture of solvent and solids yields an extract (e.g., a solution comprising compounds derived from the Amanita muscaria extract). In embodiments, the extract is concentrated. Exemplary methods of concentrating an extract include, e.g., evaporating a portion or an entirety of a solvent to create a volume of concentrated slurry at a desired concentration. In embodiments, the extract is concentrated by evaporation of the solvent (230). The evaporation can be carried out by any suitable method known to one of skill, including, for example, evaporation under reduced pressure (e.g., rotary evaporation), distillation, or by directly heating the extract to boil off the solvent.

In some embodiments, evaporation of the solvent produces a residue (e.g., a powder, or a resin) comprising compounds derived from the Amanita muscaria extract (240). This residue may be redissolved in a solvent suitable for use in vaporizer formulations, as described in various embodiments herein for base liquids. For example, the residue may be dissolved in propylene glycol (PG), vegetable glycerin (VG), or a combination thereof. In embodiments, the residue is mixed with the solvent at a fixed ratio (e.g., a fixed ratio of the weight of the residue to the volume of base liquid). In embodiments, the ratio of residue to base liquid (e.g., PG, VG) is from about 1:1 to about 1:100. In embodiments, the ratio of residue to base liquid is from about 1:1 to about 1:50. In embodiments, the ratio of residue to base liquid is from about 1:1 to about 1:20. In embodiments, the ratio of residue to base liquid is from about 1:5 to about 1:20. In embodiments, the ratio of residue to base liquid is from about 1:5 to about 1:15. In embodiments, the ratio of residue to base liquid is from about 1:5 to about 1:10. In embodiments, the ratio of residue to base liquid is from about 1:10 to about 1:20. In embodiments, the ratio of residue to base liquid is from about 1:10 to about 1:15. In embodiments, the ratio of residue to base liquid is about 1:10. For example, In embodiments, the base liquid is PG, and the ratio of residue to PG is about 1:10.

In some embodiments, the vaporizable formulation produced according to methods described herein is then packed (i.e., packaged) and labeled for commercial sale (e.g., 160, 250). In embodiments, a disclosed vaporizer formulation is packed and labeled in bulk form, for example in a bottle or vial that contains multiple doses, which may be used by individuals to fill a vaporizer device containing a refillable chamber. In some such embodiments, a disclosed vaporizer formulation is filled into a glass vial. In some such embodiments, a disclosed vaporizer formulation is filled into an amber glass vial. In embodiments, a disclosed vaporizer formulation is filled into a glass bottle. In some such embodiments, a disclosed vaporizer formulation is filled into an amber glass bottle. In embodiments, the container into which a disclosed vaporizer formulation is filled comprises a dispensing device (e.g., a pump or a dropper). In embodiments, a disclosed vaporizer formulation is packaged as in a unit dosage form. For example, In embodiments, a disclosed vaporizer formulation is packaged as a single-use or disposable chamber, such as those disclosed in embodiments herein. In embodiments, a disclosed vaporizer formulation is packaged as a disposable or single use vaporizer device as described in embodiments herein.

By way of non-limiting examples, the following formulations may be used in the devices and methods of the invention, wherein the Amanita muscaria extract is as described in embodiments herein. The A. muscaria extract may be any extract described herein, including an extract produced according to Examples 1-2, and specifically may be an AME-1 extract as described and illustrated in Tables 1-5. The following examples will be understood to be merely exemplary and not limiting of the A. muscaria extracts useful in the formulations of the present invention. One of skill will readily appreciate that any number of A. muscaria extracts can be prepared using the teachings herein in combination with the knowledge in the art, and will further understand that such extracts can be concentrated and/or standardized to create additional concentrated and/or standardized A. muscaria extracts useful in the practice of the invention, again in view of the teachings of this disclosure combined with the general knowledge of one of ordinary skill.

### Example 3: Amanita Muscaria Resin Extract

An exemplary vaporizer formulation described in embodiments herein was obtained as follows.

Solid Amanita Muscaria extract (in ground powder form) was suspended in methanol at a ratio of 1 part powder to 10 parts methanol (w/v). The suspension was mixed thoroughly for approximately 15 minutes to break apart solids. Then, the mixture was centrifuged at 15000 rpm for 5 minutes. The supernatant was removed and heated to evaporate the solvent, which yielded a gold-colored resin. This resin may be used as an extract in disclosed vaporizable formulations, for example by redissolving the resin in a vaporizable formulation base liquid. For example, in one exemplary formulation, the resin obtained by the procedure described in this Example was dissolved in propylene glycol (as the base liquid) at a ratio of 1 part resin to 10 parts propylene glycol (w/v), with heating as necessary for full dissolution of the resin. This produced a vaporizable formulation as described in embodiments herein.

Additional vaporizable formulation examples are provided below.

### Example 4:50:50 Base Liquid Formulation, 10% Amanita muscaria Extract

| Ingredient | Percentage v/v |
|---|---|
| Amanita muscaria extract | 10% |
| 50:50 base liquid (50% PG, 50% VG) | 90% |

### Example 5:30:70 Base Liquid Formulation, 10% AME-1

| Ingredient | Percentage v/v |
|---|---|
| Amanita muscaria extract | 10% |
| 30:70 base liquid (30% PG, 70% VG) | 90% |

### Example 6:70:30 Base Liquid Formulation, 10% Amanita muscaria Extract

| Ingredient | Percentage v/v |
|---|---|
| Amanita muscaria extract | 10% |
| 70:30 base liquid (70% PG, 30% VG) | 90% |

### Example 7:20:80 Base Liquid Formulation, 10% Amanita muscaria Extract

| Ingredient | Percentage v/v |
|---|---|
| Amanita muscaria extract | 10% |
| 20:80 base liquid (20% PG, 80% VG) | 90% |

### Example 8:80:20 Base Liquid Formulation, 10% Amanita muscaria Extract

| Ingredient | Percentage v/v |
|---|---|
| Amanita muscaria extract | 10% |
| 80:20 base liquid (80% PG, 20% VG) | 90% |

### Example 9: 100% PG Base Liquid Formulation, 10% Amanita muscaria Extract

| Ingredient | Percentage v/v |
|---|---|
| Amanita muscaria extract | 10% |
| 100% PG base liquid | 90% |

### Example 10: 100% VG Base Liquid Formulation, 10% Amanita muscaria Extract

| Ingredient | Percentage v/v |
|---|---|
| Amanita muscaria extract | 10% |
| 100% VG base liquid | 90% |

### Example 11:30:70 Base Liquid Formulation, 10% Amanita muscaria Extract, 5% Flavorant

| Ingredient | Percentage v/v |
|---|---|
| Amanita muscaria extract | 10% |
| Flavorant | 5% |
| 30:70 base liquid (30% PG, 70% VG) | 85% |

### Example 12:70:30 Base Liquid Formulation, 10% Amanita muscaria Extract, 5% Flavorant

| Ingredient | Percentage v/v |
|---|---|
| Amanita muscaria extract | 10% |
| Flavorant | 5% |
| 70:30 base liquid (70% PG, 30% VG) | 85% |

### Example 13:20:80 Base Liquid Formulation, 10% Amanita muscaria Extract, 5% Flavorant

| Ingredient | Percentage v/v |
|---|---|
| Amanita muscaria extract | 10% |
| Flavorant | 5% |
| 20:80 base liquid (20% PG, 80% VG) | 85% |

### Example 14:80:20 Base Liquid Formulation, 10% Amanita muscaria Extract, 5% Flavorant

| Ingredient | Percentage v/v |
|---|---|
| Amanita muscaria extract | 10% |
| Flavorant | 5% |
| 80:20 base liquid (80% PG, 20% VG) | 85% |

### Example 15: 100% PG Base Liquid Formulation, 10% A. Muscaria Extract, 5% Flavorant

| Ingredient | Percentage v/v |
|---|---|
| Amanita muscaria extract | 10% |
| Flavorant | 5% |
| 100% PG base liquid | 85% |

### Example 16: 100% VG Base Liquid Formulation, 10% A. Muscaria Extract, 5% Flavorant

| Ingredient | Percentage v/v |
|---|---|
| Amanita muscaria extract | 10% |
| Flavorant | 5% |
| 100% VG base liquid | 85% |

It should be appreciated that the above formulation examples are illustrative only. While specific proportions are provided, said proportions may be modified to include any proportion disclosed herein.

For example, the Amanita muscaria extract in the above formulation examples may be substituted for A. muscaria fungal material, or may be substituted by any of muscimol, ibotenic acid, and muscarine provided as compounds, in amounts (e.g., by % v/v, % w/v, mg/mL, etc.) determined based on the teachings herein, in combination with the general knowledge in the art.

In embodiments wherein any of muscimol, ibotenic acid, and muscarine are added as compounds, they may be present in the formulation in concentrations of between about 0.1 mg/mL and about 200 mg/mL, and in some embodiments between about 1 and about 20 mg/mL.

Such examples may also be modified to include base liquids comprising PEG in addition to, or instead of PG and VG, and further comprising water, ethanol, or a mixture thereof. In such embodiments, one of skill may modify the formulation by subtracting the volume of water from the total volume of the base liquid used in the formulation and calculating the proportion based on the result. As an example, in a 10 ml formulation wherein the base liquid comprises 9 mL of the formulation and one wishes to utilize a 30:70 PG/VG base liquid comprising 5% water, one may subtract 5% of 9 mL (0.45 mL) from the total base liquid volume (9 mL) to yield the volume of the 30:70 PG/VG mixture (8.55 mL). One may then create an 8.55 ml mixture comprising 2.565 mL of PG (30%), 5.985 mL of VG (70%), and then may add 0.45 ml of water to the mixture. The same logic can be applied to base liquids containing ethanol and base liquids containing a mixture of ethanol and water, such as vodka. One of skill may also combine the liquids to the desired proportion via the density of the liquids, which are, for example:

TABLE 8
Weight of Liquids Used in the Formulations of the Invention

| | Density |
|---|---|
| Liquid | (g/mL at 25° C.) |
| Propylene glycol | 1.036 |
| Vegetable glycerine | 1.26 |
| Polyethylene glycol | 1.27 |
| Ethanol | 0.7849 |
| Water | 1 |

Moreover, in embodiments, the Amanita muscaria extract, which may or may not be AME-1, may be obtained as a ground powder, rather than a liquid extract. In embodiments, the ground powder may be utilized in a heat-not-burn (HnB) composition, wherein the composition comprising the ground powder is heated to a sufficient degree to vaporize, but not combust.

In embodiments, fungal material such as dried Amanita muscaria mushrooms, including dried, ground and/or pulverized Amanita muscaria mushrooms, may be used in a HnB device.

Examples 4-16 may be produced by combining the ingredients according to the specific proportions of each example. It will be appreciated that the formulation of each example may be produced in any desired volume, and the ingredients may be combined with tools known to those in the art. Thus, while the following preparation examples list specific final volumes, such volumes are merely illustrative and may be altered according to methods known to those of skill.

### Example 17: Preparing a 100 mL Formulation Comprising a 50:50 PG/VG Base Liquid that Comprises 90% of the Formulation, with 10% being AME-1

A 100 mL formulation comprising 90% 50:50 PG/VG base liquid and 10% AME-1 may be produced by utilizing, e.g., a 100 mL adjustable volume pipette to combine individually 45 mL PG, 45 mL VG, and 10 mL AME-1 extract. Such formulation may be added to a chamber such as those described herein.

This example may be modified by substituting AME-1 extract with fungal material such as Amanita muscaria mushrooms, another extract thereof, or muscimol and/or muscarine. In embodiments wherein muscimol and/or muscarine are added as compounds, they may be present in the formulation in concentrations of between about 0.1 mg/ml and about 200 mg/mL, and in embodiments between about 1 mg/ml and about 20 mg/mL. In this exemplary 100 mL formulation, muscimol and/or muscarine may each be present in an amount of between 10 mg and 20,000 mg, including between 100 mg and 2,000 mg.

### Example 18: Preparing a 1 L Formulation Comprising a 30:70 PG/VG Base Liquid that Comprises 90% of the Formulation, with 10% being AME-1

A 1 L formulation comprising a 90% 30:70 PG/VG base liquid and 10% AME-1 may be produced by individually combining 270 mL PG, 630 mL VG, and 100 ml of AME-1 extract in a container suitable for such volumes. The formulation may then be agitated to form a uniform mixture, which may be done by an operator, or via paddles, arms, or other agitation means within the container.

This example may be modified by substituting AME-1 extract with fungal material such as Amanita muscaria mushrooms, another extract thereof, or muscimol and/or muscarine. In embodiments wherein muscimol and/or muscarine are added as compounds, they may be present in the formulation in concentrations of between about 0.1 mg/ml and about 200 mg/mL, and in some embodiments between about 1 mg/ml and about 20 mg/mL. In this exemplary 1 L formulation, muscimol and/or muscarine may each be present in an amount of between 0.1 g and 200 g, including between 1 g and 20 g.

### Example 19: Preparing a 1,000 L Formulation Comprising a 80:20 PG/VG Base Liquid that Comprises 90% of the Formulation, with 10% being AME-1

A 1,000 L formulation comprising 90% 80:20 PG/VG base liquid and 10% AME-1 may be produced by individually combining 720 L of PG, 180 L of VG, and 100 L of AME-1 extract in a container suitable for such volumes. The formulation may then be agitated to form a uniform mixture, which may be done by an operator, or via paddles, arms, or other agitation means within the container, including by using a commercial or industrial mixer.

This example may be modified by substituting AME-1 extract with fungal material such as Amanita muscaria mushrooms, another extract thereof, or muscimol and/or muscarine. In embodiments wherein muscimol and/or muscarine are added as compounds, they may be present in the formulation in concentrations of between about 0.1 mg/ml and about 200 mg/ml, and in some embodiments between about 1 mg/ml and about 20 mg/mL. In this exemplary 1,000 L formulation, muscimol and/or muscarine may each be present in an amount of between 100 g and 200,000 g, including 1,000 g and 20,000 g.

In some embodiments, the vaporizer formulation further comprises an additional composition. In embodiments, the additional composition comprises tobacco, an extract from tobacco, or nicotine. In embodiments, the additional composition is cannabis, a cannabis concentrate, or a cannabis extract. In embodiments, the cannabis extract is a cannabis oil, a cannabis distillate, or a cannabis isolate. In embodiments, the cannabis extract is a full spectrum cannabis extract or a broad spectrum cannabis extract. In embodiments, the cannabis extract is from cannabis plants high in THC. In embodiments, the cannabis extract is from cannabis plants high in CBD. In embodiments, the cannabis extract is from cannabis plants low in THC. In embodiments, the cannabis extract is free from THC, or is substantially free from THC. In embodiments, the cannabis extract is an extract from industrial hemp. In embodiments, the additional composition comprises a cannabinoid.

As used herein, the term “cannabinoid” includes all naturally occurring phytocannabinoids. Naturally-occurring phytocannabinoids in Cannabis plants include THC and CBD among numerous others, including as non-limiting examples, cannabinol (CBN), cannabigerol (CBG), cannabinodiol (or cannabinodiol) (CBDL, CBND), cannabichromene (CBC), cannabielsoin (CBE), cannabicyclol (CBL), cannabicitran (CBT), cannabivarin (CBV), cannabidivarin (CBDV), tetrahydrocannabivarin (THCV), cannabichromevarin (CBCV), cannabigerovarin (CBGV), and cannabigerol monomethyl ether (CBGM).

The term “cannabinoid” also includes cannabinoid acids. “Cannabinoid acids” refers generally to a heterogenous group of naturally-occurring compounds found within the cannabis plant that are the carboxylic acid precursors of their corresponding “neutral” cannabinoid (see, e.g., Burstein, S. Bioorg. Med. Chem. 2014, 22 (10), 2830-2843). Non-limiting examples of cannabinoid acids include, among others, cannabichromenic acid (CBCA), cannabichromevarinic acid (CBCVA), cannabidiolic acid (CBDA), cannabidiolic acid monomethyl ether (CBDMA), CBDPA (cannabidiphorolic acid), CBDVA (cannabidivarinic acid), CBFA (cannabifuranic acid), CBEA (cannabielsoic acid), CBGA (cannabigerolic acid), CBGVA (cannabigerovarinic acid), CBLA (cannabicyclolic acid), CBNA (cannabinolic acid), CBNDA (cannabinodiolic acid), CBTA (cannabitriolic acid), THCA (tetrahydrocannabinolic acid, including 48 and A9 THCA), THCPA (tetrahydrocannabipgorolic acid), and THCVA (tetrahydrocannabivarinic acid). “Neutral cannabinoids” refer to the decarboxylated form of a given cannabinoid acid; i.e., the cannabinoid where the additional carboxyl group is removed. Without being bound by theory, decarboxylation generally is a function of pressure, temperature, and time, and typically occurs when a given cannabinoid acid is exposed to heat.

As it relates to the invention, where such inclusion is technically meaningful, each cannabinoid acid listed herein, i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, will be understood to include their isomers, such as structural isomers and stereoisomers (including enantiomers), the −A and −B isomers for each cannabinoid acid, double bond isomers, and other such isomers known to those of skill. Thus, “THCA” will, in some embodiments, be understood to include THCA-A and THCA-B. In some embodiments, included among cannabinoids are the cannabinoid carboxylic acids and their carboxylate salts (see, e.g., U.S. Pat. No. 9,376,367). Accordingly, reference to a cannabis extract includes, in such embodiments, cannabinoid carboxylic acid-rich extracts and other extracts containing cannabinoid carboxylic acids, as well as the crystalline salts of cannabinoid carboxylic acids.

“Cannabinoids” will be as described above and as generally known to those in the art, for example as set forth and described by Radwan et al. Molecules 2021:26(9); 2774 (Radwan 2021), as an exemplary and non-limiting categorization, which is incorporated by reference as if fully set forth herein. Without being bound by theory, cannabinoids according to the categorization of Radwan 2021 include compounds with a characteristic C21 terpenophenolic backbone that are part of one of 11 cannabinoid sub-classes, namely: cannabichromene (CBC)-type, cannabidiol (CBD) type, cannabielsoin (CBE) type, cannabigerol (CBG) type, cannabicyclol (CBL) type, cannabinol (CBN) type, cannabinodiol (CBND) type, cannabitriol (CBT) type, (−)-Δ⁸-trans-tetrahydrocannabinol (Δ⁸-THC) type, (−)-Δ⁹-trans-tetrahydrocannabinol (Δ⁹-THC) type, and miscellaneous-type cannabinoids. Non-limiting examples of such cannabinoids, which will be understood to be useful in the practice of the invention, are known by reference to the disclosure of Radwan 2021 and the below. In some embodiments, a disclosed formulation comprises any one or more of such cannabinoids.

In some embodiments, the cannabinoid is any of a Δ⁹-THC-type cannabinoid, a Δ⁸-THC-type cannabinoid, a CBG-type cannabinoid, a CBD-type cannabinoid, a CBND-type cannabinoid, a CBE-type cannabinoid, a CBL-type cannabinoid, a CBN-type cannabinoid, a CBC-type cannabinoid, a CBT-type cannabinoid, and a miscellaneous-type cannabinoid. In some embodiments, Δ⁹-THC-type cannabinoids include Δ⁹-THC-C5, Δ⁹-THCAA-C5, Δ⁹-THCAB-C5, Δ⁹-THC-C4, Δ⁹-THCAA-C4, Δ⁹-THCV, Δ⁹-THCVAA, Δ⁹-THCO, Δ⁹-THCOAA, Δ⁹-THC-aldehyde, β-fenchyl (−)-Δ⁹-trans-tetrahydrocannabinolate, α-fenchyl (−)-Δ⁹-trans-tetrahydrocannabinolate, epi-bornyl (−)-Δ⁹-trans-tetrahydrocannabinolate, bornyl (−)-Δ⁹-trans-tetrahydrocannabinolate, α-terpenyl (−)-Δ⁹-trans-tetrahydrocannabinolate, 4-terpenyl (−)-Δ⁹-trans-tetrahydrocannabinolate, α-cadinyl (−)-Δ⁹-trans-tetrahydrocannabinolate, γ-eudesmol (−)-Δ⁹-trans-tetrahydrocannabinolate, 8α-hydroxy-(−)-Δ⁹-trans-tetrahydro-cannabinol, 8β-hydroxy-(−)-Δ⁹-trans-tetrahydro cannabinol, 11-acetoxy-(−)-Δ⁹-trans-tetrahydrocannabinolic acid A, 8-oxo-(−)-Δ⁹-trans-tetrahydrocannabinol, cannabisol, (−)-Δ⁹-trans-tetrahydro-cannabi-phorol, and (−)-Δ⁹-trans-tetrahydrocannabihexol. In some embodiments, Δ⁸-THC-type cannabinoids include Δ⁸-THC, Δ⁸-THCA, 10α-OH-Δ⁸-THC, 10β-OH-Δ⁸-THC, and 10a-α-hydroxy-10-oxo-Δ⁸-THC. In some embodiments, CBG-type cannabinoids include (E) CBG, (E) CBGA, (E) CBGG, (E) CBGAM, (E) CBGV, (E) CBGVA, (Z) CBGA, 5-acetyl-4-hydroxy-cannabigerol, (±)-6,7-trans-epoxycannabigerolic acid, (±)-6,7-cis-epoxycannabigerolic acid, (±)-6,7-cis-epoxycannabigerol, (±)-6,7-trans-epxoycannabigerol, camagerol, and sesquicannabigerol. In some embodiments, CBD-type cannabinoids include CBD-C₅, CBDA-C₅, CBDM-C₅, CBD-C₄, CBDV, CBDVA, CBD-C₁, CBDH, CBDP, and CBDD. In some embodiments, CBND-type cannabinoids include CBND-C₃ and CBND-C₅. In some embodiments, CBE-type cannabinoids include CBE-C₅, CBEAA-C₅, CBEAB-C₅, CBE-C₃, and CBEAB-C₃. In some embodiments, CBL-type cannabinoids include CBL, CBLA, and CBLV. In some embodiments, CBC-type cannabinoids include CBC, CBCA, ±CBCV, ±CBCV, CBCVA, 4-acetoxy-CBC, (±)-3″-hydroxy-Δ4″-cannabichromene, (−)-7-hydroxy-cannabichromene, and CBC-C₃. In some embodiments, CBN-type cannabinoids include CBN-C₅, CBNA-C₅, CBN-C₄, CBN-C₃, CBN-C₂, CBN-C₁, CBNM-C₅, 8—OH—CBN, 8—OH—CBNA, 1'S—OH—CBN, and 4-terpenyl-cannabinolate. In some embodiments, CBT-type cannabinoids include (−)-trans-CBT-C₅, (+)-trans-CBT-C₅, (+)-cis-CBT-C₅, (+)-trans-CBT-C₃, CBT-C₃-homologue, (−)-trans-CBT-OEt-C₅, (−)-trans-CBT-OEt-C₃, 8,9-Di-OH—CBT-C₅, and CBDA-C₅, and 9—OH—CBT-C₅ ester. In some embodiments, miscellaneous-type cannabinoids include DCBF-C₅, CBF-C₅, OH-iso-HHCV-C₃ OTHC, cannabicitran, cis-Δ⁹-THC, CBCON-C₅, CBR, CBTT, CBCN-C₅, CBCN-C₃, cis-iso-Δ⁷-THCV, trans-iso-Δ⁷-THCV, trans-iso-Δ⁷-THC, CBCNB, CBCNC, CBCND, (−)-(7R)-cannabicoumarononic acid, 4-acetoxy-2-geranyl-5-hydroxy-3-n-pentylphenol, 2-geranyl-5-hydroxy-3-n-pentyl-1,4-benzoquinone, 5-acetoxy-6-geranyl-3-n-pentyl-1,4-benzoquinone, CBM, CBX, 10α-hydroxy-Δ^{9,11}-hexahydrocannabinol, 9,10β-epoxyhexahydrocannabinol, 9α-hydroxyhexahydro-cannabinol, 7-oxo-9α-hydroxyhexahydrocannabinol, 10α-hydroxyhexahydrocannabinol, 10αR-hydroxy-hexahydrocannabinol, and 9α-hydroxy-10-oxo-Δ^{6a,10a}-THC.

In some embodiments, the cannabinoid is THC. In embodiments, the THC is delta-9 THC. In embodiments, the THC is delta-8 THC. In embodiments, the cannabinoid is CBD. In embodiments, the cannabinoid is one or more of cannabinol (CBN), cannabichromene (CBC), cannabigerol (CBG), tetrahydrocannabinolic acid (THCA), cannabidiolic acid (CBDA), tetrahydrocannabivarin (THCV), and cannabidivarin (CBDV).

In some embodiments, the vaporizer formulation further comprises an additional compound. In embodiments, the additional compound is nicotine. In embodiments, the additional compound is CBD. In embodiments, the additional compound is THC. In embodiments, the THC is delta-9 THC. In embodiments, the THC is delta-8 THC. In embodiments, the additional compound is a cannabinoid other than CBD and THC. In embodiments, the cannabinoid is one or more of cannabinol (CBN), cannabichromene (CBC), cannabigerol (CBG), tetrahydrocannabinolic acid (THCA), cannabidiolic acid (CBDA), tetrahydrocannabivarin (THCV), and cannabidivarin (CBDV), including combinations thereof.

In some embodiments, the additional compound is a terpene. Terpenes are major components of the essential oils present in various plants, including Cannabis. Terpenes are a significant component of the distinctive aromas and flavors, and may themselves have psychoactive properties or contribute to the biological effects of formulations, including disclosed vaporizer formulations. In embodiments, the terpene is one or more of pinene (e.g., alpha-pinene, beta-pinene), limonene, linalool, caryophyllene, caryophyllene oxide, myrcene, humulene, borneol, eucalyptol, terpineol, nerolidol, phytol, geraniol, bisabolol, bisabolene, camphene, beta-amyrin, thujone, citronellol, pulegone, cycloartenol, cymene, sabinene, carene, terpinene, fenchol, isopulegol, guaiol, phellandrene, eudesmol, ocimene, cadinene, elemene, gurjunene, farnesene, friedelin, carvacrol, eugenol, camphor, menthol, iso-menthone, neral, gerial, viridiflorol, germacrene, thymol, menth-2-en-1-ol, farnesol, carotol, myrtenol, terpinolene, valencene, cedrene, pinenesene, and squalene, including combinations thereof.

In some embodiments, the vaporizer formulation comprises a psychedelic as an additional compound. Broadly, and without intending to be limited by theory, “psychedelic” compounds are compounds that induce altered states of consciousness, characterized by perceptual and cognitive distortions, changes in mood, and often profound changes in thought and self-perception. Psychedelics are typically used for their psychoactive effects, which can have therapeutic, spiritual, or recreational applications. Psychedelics include naturally-occurring substances such as N,N-dimethyltryptamine (DMT), as well as synthetic compounds like 2-5-dimethoxy-4-bromophenethylamine (2C—B). For naturally-occurring substances, the term “psychedelic” may encompass the psychedelic compound itself (e.g., psilocin or DMT), any fungal or plant sources thereof (e.g., mushrooms of the Psilocybe genus, root bark of Mimosa hostilis), as well as fungal or plant extracts (e.g., aqueous extracts, alcoholic extracts), as will be understood depending on context. In some embodiments, a psychedelic may be obtained, isolated, and/or purified from a natural source, may be chemically synthesized, may be produced by biosynthesis, as well as combinations thereof (e.g., semisynthesis or partial chemical synthesis from a natural isolate). In some embodiments, the psychedelic is a tryptamine, phenethylamine, or lysergamide, such compounds being generally known in the art (Shulgin A, Shulgin A. PiHKAL: A Chemical Love Story. Berkeley, CA: Transform Press; 1991; Shulgin A, Shulgin A. TiHKAL: The Continuation. Berkeley, CA: Transform Press; 1997; Grob C S, Grigsby J. Handbook of Medical Hallucinogens. New York, NY: Guilford Press; 2021; Nichols D E. Psychedelics. Pharmacological Reviews. 2016; 68(2):264-355; Glennon R A. Arylalkylamine Drugs of Abuse: An Overview of Drug Discrimination Studies. Pharmacology Biochemistry and Behavior. 1999; 64:251-256; each of which is incorporated by reference as if fully set forth herein).

In some embodiments, the psychedelic included in a disclosed vaporizer formulation is a tryptamine. As will be readily understood by those in the art, tryptamines are compounds having the general structure below, wherein R^{N1}, R^{N2}, R^{α}, R^{β}, R², R⁴, R⁵, R⁶, and R⁷ are as defined herein and as generally understood in the art:

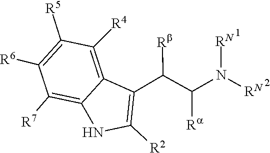

In some embodiments, R^{N1}, R^{N2}, R^{α}, R^{β}, R², R⁴, R⁵, R⁶, and R⁷ are each independently hydrogen, deuterium, halogen (F, Cl, Br, or I), OH, phosphoryloxy, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. Additionally, any two of R^{N1}, R^{N2}, R^{α}, R^{β}, R², R⁴, R⁵, R⁶, and R⁷ and the intervening atoms can be taken together to form an optionally substituted optionally substituted cycloalkyl, optionally substituted cycloalkenyl, optionally substituted aryl, or optionally substituted heterocyclyl. In some embodiments, the tryptamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, a disclosed vaporizer formulation comprises DMT. In some embodiments, a disclosed vaporizer formulation comprises 5-methoxy-N,N-dimethyltryptamine (5-MeO-DMT).

In some embodiments, the psychedelic included in a disclosed vaporizer formulation is a phenethylamine. As will be readily understood by those in the art, phenethylamines are compounds having the general structure below, wherein R^{N1}, R^{N2}, R^{α}, R^{β}, and each of R²—R⁶ are as taught herein and as generally understood in the art:

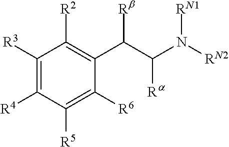

In some embodiments, R^{N1}, R^{N2}, R^{α}, R^{β}, and each of R²-6 are independently hydrogen, deuterium, halogen, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. In some embodiments, R³ and R⁴ are joined together to form an optionally substituted heterocyclyl, such as a dioxole (as with MDMA), a furan, a tetrahydrofuran, a thiophene, a pyrrole, a pyridine, a pyrrolidine, an ethylene oxide, an ethylenimine, a trimethylene oxide, a pyran, a piperidine, an imidazole, a thiazole, a dioxane, a morpholine, or a pyrimidine. In some embodiments, R³ and R⁴ are joined together to form an optionally substituted aryl, such as a phenyl. In some embodiments, the phenethylamine comprises a quaternary ammonium cation wherein each of R^{N1}, R^{N2}, and an additional R^{N3} are independently an alkyl group or an aryl group, and with all other substituents as above. In some embodiments, the phenethylamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, a disclosed vaporizer formulation comprises 4-bromo-2,5-dimethoxyphenethylamine (2C—B), 4-benzyloxy-3,5-dimethoxy-amphetamine (3C—BZ), 4-chloro-2,5-dimethoxyphenethylamine (2C—C), 2,5-dimethoxy-4-methyl-phenethylamine (2C-D), 2,5-dimethoxy-4-ethyl-phenethylamine (2C-E), 3,5-dimethoxy-4-ethoxyamphetamine (3C-E), 2,5-dimethoxy-4-fluorophenethylamine (2C—F), 2,5-dimethoxy-3,4-dimethylphenethylamine (2C-G), 2,5-dimethoxy-3,4-trimethylene-phenethylamine (2C-G-3), 2,5-dimethoxy-3,4-tetramethylenephenethylamine (2C-G-4), 3,4-norbornyl-2,5-dimethoxyphenethylamine (2C-G-5), 1,4-dimethoxynaphthyl-2-ethylamine (2C-G-N), 2,5-dimethoxyphenethylamine (2C—H), 4-iodo-2,5-dimethoxyphenethylamine (2C—I), 2,5-dimethoxy-4-nitro-phenethylamine (2C—N), 2,5-dimethoxy-4-isopropoxyphenethylamine (2C—O-4), 2,5-dimethoxy-4-propylphenethylamine (2C—P), 4-cyclopropylmethoxy-3,5-dimethoxyphenethylamine (CPM), 2,5-dimethoxy-4-methylselenophenethylamine (2C-SE), 2,5-dimethoxy-4-methylthiophenethylamine (2C-T), 2,5-dimethoxy-4-ethylthiophenethylamine (2C-T-2), 2,5-dimethoxy-4-isopropylthiophenethylamine (2C-T-4), 2,6-dimethoxy-4-isopropylthiophenethylamine (psi-2C-T-4), 2,5-dimethoxy-4-propylthiophenethylamine (2C-T-7), 4-cyclopropylmethylthio-2,5-dimethoxyphenethylamine (2C-T-8), 4-(t)-butylthio-2,5-dimethoxy-phenethylamine (2C-T-9), 2,5-dimethoxy-4-(2-methoxyethylthio) phenethylamine (2C-T-13), 4-cyclopropylthio-2,5-dimethoxyphenethylamine (2C-T-15), 4-(s)-butylthio-2,5-dimethoxyphenethylamine (2C-T-17), 2,5-dimethoxy-4-(2-fluoroethylthio) phenethylamine (2C-T-21). In embodiments, a disclosed vaporizer formulation comprises 2C—B. In embodiments, a disclosed vaporizer formulation comprises 2C—C. In embodiments, a disclosed vaporizer formulation comprises 2C-D. In embodiments, a disclosed vaporizer formulation comprises 2C-E. In embodiments, a disclosed vaporizer formulation comprises 2C—I. In embodiments, a disclosed vaporizer formulation comprises 2C-T. In embodiments, a disclosed vaporizer formulation comprises a 2C-T analog (i.e., 2C-T-2, 2C-T-4, 2C-T-7, 2C-T-8, 2C-T-9, 2C-T-21, etc.).

The disclosed formulations may also be used with any vaporizer device, whether it be a vaporizer device that is mouth-to-lung or direct-to-lung, a device that uses single-use pods, a device that uses disposable pods, a device that uses refillable pods, a modified or “mod” pod device, including a closed pod system and an open pod system; a pen device that can be refilled with the formulations disclosed herein, including simple refillable pens, such as fixed voltage pens; a device having a vape cartridge or cart, e.g., standard 0.5 mL or 1.0 mL cartridges, variable voltage pens, and variable temperature pens; and modified pens (mods) that are custom-crafted by the user, including regulated mods (containing a circuit board) and unregulated mods (not containing a circuit board), tube mods, box mods, and mechanical mods (mechs); a disposable, single-use pen device; other “vape pens;” an e-cigarette device, a rechargeable e-cigarette device, a cigalike device, including a disposable cigalike and a refillable cigalike; an e-cigar, an e-pipe, a heat-not-burn device, and any other such vape devices known to those of skill. Several exemplary such devices are disclosed in more detail below.

### E. Vaporizer Devices

In some aspects are provided vaporizer devices comprising the disclosed formulations. In some embodiments, a vaporizer device comprises a mouthpiece, a heating element, a power source, and a chamber comprising a formulation disclosed herein.

In embodiments, the device comprises a mouthpiece. In embodiments, the mouthpiece may be any mouthpiece made of any solid material with an airway passage by which the user may be supplied a vapor comprising the vaporizer formulations disclosed herein. In embodiments, the solid material may be any of plastic, glass, metal, wood, or a combination thereof. In embodiments, the mouthpiece may be removable. In embodiments, the mouthpiece may be non-removable. In embodiments, the mouthpiece may be connected to the device directly, or may be connected via an extending member, such as a hose. In embodiments, the mouthpiece has an extension piece protruding from the mouthpiece; which may be constructed of any of glass, metal, wood, plastic, rubber, or a combination thereof. In embodiments, the extension piece may be removable. In embodiments, the mouthpiece is connected to a chamber capable of holding the formulations disclosed herein, such that when the mouthpiece is removed, the chamber is also removed. In embodiments, the mouthpiece is contoured for a user's mouth (including where the mouthpiece is specially designed for a specific user). In embodiments, the mouthpiece contains no such contouring.

In embodiments, the device comprises a heating element. In embodiments, the heating element includes any such means of heating the formulations disclosed herein sufficient to vaporize at least a portion of the vaporizer formulations (the heating element may also be referred to as an atomizer). Vape heating elements generally comprise a coil in contact with a power unit, and may or may not include a wicking material. In embodiments, the wicking material may include any of cotton, wood, metal, ceramic, polymer, or other materials suitable for absorbing the vaporizable formulation. In embodiments, the heating element may include any of metal, glass, ceramic, or quartz.

In embodiments, the heating element contains a coil of a given gauge (thickness), which will affect the resistance (measured in ohms), and the ramp-up time (the time it takes the coil to generate enough heat to supply the user with the formulation). In embodiments, the coil gauge may be any of 10, 12, 14, 16, 18, 20, 22, 24, 26, 28, 30, 32, 34, 36, 38, 40, 42, 44, 46, 48, and 50 gauge. In embodiments, the resistance of the wire is between about 0.1 and 6 ohm, inclusive.

In embodiments, the heating element comprises a wicking material. In embodiments, the wicking material is any of silica, cotton, including organic cotton; cellucotton, including cotton cellucotton and rayon cellucotton; EKOWOOL™ or other cellulose fiber, and stainless steel mesh. That said, the wicking material may be any flammable material known to those of skill capable of absorbing the disclosed formulations.

In embodiments, the heating element is connected to a power source. In embodiments, the power source is a battery. In embodiments, the battery is an internal battery within the device. In embodiments, the battery is an external battery connected to the device. In embodiments, the battery is a rechargeable battery. In embodiments, the battery is a replaceable battery. In embodiments, the battery is not rechargeable or replaceable (e.g., a single use device).

In some embodiments, the vaporizer further comprises a means for controlling the device. In embodiments, a user is able to cause the battery to apply a power and a voltage to the heating element by actuating a button, switch, knob, or any other such actuating input known to those of skill. In embodiments, the user can control either or both of the power and the voltage applied to the heating element. In embodiments, the user controls the power and/or voltage with one or more buttons on the exterior of the device. In embodiments the user controls the power and/or voltage with one or more touch sensitive elements, such as a screen. In embodiments, the vaporizer further comprises means for displaying information. In embodiments, the device includes a graphical user interface that illustrates to the user the power and/or voltage being applied to the heating element. In embodiments, the graphical user interface allows touch input from the user to select a given power, voltage, and/or mode of operation. In embodiments, the graphical user interface is a screen. In embodiments, the graphical user interface is an LED array. In embodiments, the device further comprises a number of censors configured to monitor and communicate information such as temperature, pH, volume, or a combination thereof. In embodiments, the sensors communicate said information to the user through the screen and/or the LED array.

The heating element will heat the formulations disclosed herein to a degree sufficient to vaporize the formulations. In embodiments wherein the fungal extract is obtained as a ground powder, the heating element will heat the formulation to a degree sufficient to supply the user with the ingredients of the fungal extract without combusting the extract (e.g., in heat-not-burn devices). In embodiments, a heat-not-burn (HnB) or like device is used to vaporize dried fungus, including dried A. muscaria mushrooms, directly. Such dried mushrooms generally will be crushed, ground, and/or pulverized before use in the HnB device.

In embodiments, the device comprises a chamber comprising a formulation disclosed herein. In embodiments, the device is obtained by a user with the formulation contained within the device.

In such embodiments, the device is a single use device. In embodiments, the device is a device wherein the individual may refill the chamber with a formulation or replace the chamber with a chamber filled with a formulation.

In embodiments, the chamber is a refillable chamber, including a refillable chamber in a pen, a mod device, including a mech, a regulated mod, an unregulated mod, a tube mod, and a box mod; a refillable cigalike, an e-pipe, or a mod pod device that utilizes refillable pods.

In embodiments, the chamber is a removable chamber, including a single use container, such as a single use pod or single use cartridge.

In embodiments, the chamber is a refillable chamber and a removable chamber, including a removable and refillable pod, such as those for a mod pod device; or in a removable and refillable cartridge.

In embodiments, a user may purchase the formulation separately from the device, e.g., as a liquid formulation (including any of the formulations disclosed herein) to be added to a chamber.

In embodiments, the formulation is contained within a removable chamber within the device. In embodiments, the removable chamber may be a single use container, such as those described above. In embodiments, the formulation is contained within a refillable chamber within the device. In embodiments, the formulation is contained within a refillable chamber and a removable chamber, such as those described above.

In embodiments, the device is constructed of a solid material, including any of glass, metal, wood, plastic, rubber, paper, cardboard, or any other such solid material known to those of skill.

In some aspects are chambers for use with a vaporizer device, comprising a disclosed formulation. In embodiments, the chamber is refillable. In embodiments, the chamber is removable. In embodiments, the chamber is refillable and removable. In embodiments, the removable chamber is a single use container.

### F. Methods of Use

a. Methods of Modulating Neurotransmission

In some aspects, provided are methods of using the disclosed A. muscaria formulations to modulate neurotransmission. In some embodiments, the disclosed A. muscaria formulations are used in the manufacture of a medicament to modulate neurotransmission. In embodiments, the disclosed A. muscaria formulations are administered, e.g., in a therapeutically effective amount, to a subject to modulate neurotransmission in said subject. In embodiments, modulating neurotransmission promotes health and wellness. In embodiments, modulating neurotransmission results in an improvement, such as clinical improvement, of a condition, such as a disease or a disorder.

Detecting a change in neurotransmitter levels in a subject, such as an increase or a decrease, can be achieved according to methods known to one of skill, for example, brain microdialysis (Chefer et al., Curr Protoc Neurosci. 2009; Chapter: Unit 7.1; Darvesh et al., Expert Opin Drug Discov. 2011; 6(2):109-127) and brain imaging, for example, positron emission tomography (PET) and single photon emission computed tomography (SPECT) (see e.g., Wong & Gjedde, Encyclopedia of Neuroscience, 2009; 939-952 and Takano, Front Psychiatry, 2018; 9:228).

In embodiments, the modulation or regulation of neurotransmission is that of gabaminergic or cholinergic neurotransmission. In embodiments, the modulation or regulation of gabaminergic or cholinergic neurotransmission is without significant modulation or regulation of glutaminergic neurotransmission. In embodiments, the modulation or regulation of neurotransmission is without significant modulation or regulation of glutaminergic neurotransmission because of a lack of, only trace amounts of, or a relatively low amount of, ibotenic acid. In embodiments, the modulation or regulation of neurotransmission is without significant modulation or regulation of glutaminergic neurotransmission caused by ibotenic acid.

The dysregulation of neurotransmitters has been shown to cause certain diseases and disorders. For example, when the GABAA system is dysregulated, the clinical manifestation is often anxiety. GABAA receptors also regulate nerve activity, and thus plays a role in pain stimulation. Muscimol is a potent, selective GABAA agonist and therefore may function as a regulator of gabaminergic neurotransmission, and may also alleviate pain. GABAA receptors are ion channels which can be activated by the neurotransmitter GABA, or by drugs (e.g., muscimol). When these channels open they are permeable to negatively charged chloride ions (Cl⁻). As a result, muscimol can either inhibit or stimulate nerves, depending on the relative concentration of Cl⁻ inside and outside the cell. Accordingly, GABAA receptors are extremely important for regulating nerve activity throughout the nervous system. When dysregulated, an individual often experiences anxiety. GABAA also regulates nerve activity and plays a role in pain stimulation. Most depressant and sedative drugs such as benzodiazepine tranquilizers, barbiturates, anesthetics, and alcohol are believed have a modulatory effect on the GABAA receptor by binding to allosteric sites on GABAA receptors where they enhance the actions of GABA in accumulating negatively charged chloride ions into the cell, inducing sedative or anesthetic effects. Long-term administration of benzodiazepines results in the development of tolerance to some of the effects, reducing their clinical efficacy. In embodiments, the disclosed A. muscaria formulations offer significant benefits versus other gabaminergic compounds like benzodiazepines and barbiturates. In embodiments, administration of a disclosed A. muscaria composition results in binding and activation of GABAA receptors.

Muscarine mimics the action of the neurotransmitter acetylcholine by agonizing muscarinic acetylcholine receptors, which modulates cholinergic neurotransmission. There are five different types of muscarinic receptors: M1, M2, M3, M4 and M5. The M2 and M3 subtypes mediate muscarinic responses at peripheral autonomic tissues; M1 and M4 subtypes are more abundant in brain and autonomic ganglia. M1, M3, and M5 interact with Gq proteins to stimulate phosphoinositide hydrolysis and the release of intracellular calcium. M2 and M4, by contrast, interact with Gi proteins to inhibit adenylyl cyclase, which results in a decrease of intracellular concentration of cyclic adenosine monophosphate (CAMP). Muscarinic receptors also signal via other pathways, for instance via G beta-gamma complex modulation of potassium channels. This allows muscarine to modulate cellular excitability via the membrane potential.

In some embodiments, use of a disclosed vaporizer formulation such as in a disclosed vaporizer device will result in modulation or regulation of gabaminergic and/or cholinergic neurotransmission. In embodiments, the modulation or regulation of such neurotransmission via the disclosed methods will improve health and wellness, such as described below. In embodiments, the modulation or regulation of such neurotransmission via the disclosed methods results in mild euphoria, such as described below.

In some embodiments, the methods comprise heating the vaporizer formulations with the vaporizer devices to vaporize at least a portion of the vaporizer formulations. In embodiments, when vaporized, the portion of the vaporizer formulations becomes a vapor that may include a mist or an aerosol that includes fine solid and/or liquid particles suspended in a gas or, in embodiments, the vapor may be substantially formed as a gas. The vapor may also include a gaseous substance having small droplets of oil, water and/or other chemical compounds suspended therein. In some cases, a vapor includes liquid (e.g., water, oil, etc.) particles mixed with hot ambient air, which is cooled down so as to condense into a fine cloud of visible airborne droplets.

In some embodiments, a user of a vaporizer device in which at least a portion of the vaporizer formulation is vaporized will inhale the vaporized portion, that is, at least some quantity of the vaporized portion, such as through the mouthpiece of the device.

In some embodiments, a disclosed formulation, including as delivered via a disclosed device, such as when inhaled by a user through the mouthpiece of the device, are useful for modulating or regulating neurotransmission. In embodiments, when so inhaled, the formulations will modulate or regulate neurotransmission.

b. Methods of Improving Health and Wellness

In some aspects are methods of using a vaporizer formulation or a vaporizer device disclosed herein to improve health and wellness. Improvements to “health and wellness” will include such improvements to health and improvements to wellness as will be readily appreciate in the art, and include improvements to general feelings of well-being, such as increases in or increased time spent in a state of being comfortable, content, at peace, calm, unstressed, healthy, happy, joyful, uplifted, and other such positive states, feelings, and moods. In some embodiments, an improvement to health and happiness is a state, feeling, or mood of euphoria, including of mild euphoria. In some embodiments, the methods of using a vaporizer formulation or a vaporizer device result in an improvement to health and wellness.

In some embodiments, improving health and wellness comprises eliciting or enhancing calmness in a subject. In embodiments, the disclosed A. muscaria formulations are used to elicit or enhance calmness in a subject. In embodiments, disclosed is use of A. muscaria formulations to elicit or enhance calmness. In embodiments, disclosed is use of A. muscaria formulations in the manufacture of a medicament to elicit or enhance calmness. In embodiments, administering the disclosed A. muscaria formulations to a subject elicits or enhances calmness in said subject.

In some embodiments, eliciting or enhancing calmness in a subject comprises a reduction in any of anxiety, worry, and hopelessness. In embodiments, eliciting or enhancing calmness in a subject comprises promoting feelings of calm, tranquility, and a sense of peace. Physiological responses to eliciting or enhancing calmness in a subject may include a reduction in blood pressure and heart rate, as well as other signs of calmness known to those in the art

In some embodiments, improving health and wellness comprises reducing stress in a subject. In embodiments, reducing stress comprises a reduction in any of anxiety, worry, and hopelessness. In embodiments, reducing stress comprises promoting feelings of calm, tranquility, and a sense of peace. Physiological responses to a reduction in stress may include a reduction in blood pressure and heart rate, as well as other signs of a reduction of stress known to those in the art.

In some embodiments, the disclosed A. muscaria composition provides methods for improving a sense of calm and elicits feelings of comfortability, tranquility, and relaxation. Improvements in feeling a sense of calm may include a state of mind being free from agitation, excitement or disturbance. Additionally, improvements in a sense of calm may include improvements in emotional regulation and an increased sense of acceptance of self and of others (see Kraus et al., Social Indicators Research. 2008; 92, 169-181; Juneau et al., Peerj. 2020; 1-19). Measurements of such will be readily understood and appreciated according to ordinary skill. Exemplary measures of improvements in sense of calm include the Equanimity Scale (the EQUA-S), Brief Mood Introspection Scale (BMIS) (Juneau et al., Peerj. 2020 1-19; Mayer et al., Journal of Personality and Social Psychology. 1988; 102-111).

In some embodiments, improving health and wellness comprises promoting restorative sleep, including any of waking up feeling refreshed, increasing the amount of time spent in deep sleep, increasing the quantity of peaceful dreams, and reducing the amount of sleep necessary to feel refreshed, as well as other signs of a promotion of restorative sleep as will be known to those in the art. In embodiments, improving health and wellness comprises preventing insomnia. In embodiments, improving health and wellness comprises preventing insomnia. In embodiments, improving health and wellness comprises reducing the severity of insomnia. The severity of insomnia may be determined, e.g., with use of The Insomnia Severity Index (ISI), a brief self-report instrument measuring the subject's perception of both nocturnal and diurnal symptoms of insomnia.

In some embodiments, improving health and wellness comprises any of a soothing of the body, a calming of the mind, and a reduction in physical distress; including feeling relaxed, at peace, and content; and a decrease in aches, pains, numbness, and tingling.

In some embodiments, improving health and wellness comprises includes any one or more of a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction.

In embodiments, the improvement to health and wellness is a reduction in stress, including any of a reduction in anxiety, worry, and hopelessness; a promotion of feelings of calm, tranquility, and a sense of peace; and a reduction in blood pressure and heart rate, as well as other signs of a reduction of stress as will be known to those in the art.

In embodiments, the improvement to health and wellness is an easing of muscular tension, including any of a reduction in soreness, tightness, aches, and pains; and an increase in flexibility and range of motion, as well as other signs of an easing of muscular tension as will be known to those in the art.

In embodiments, the improvement to health and wellness is a promotion of restorative sleep, including any of waking up feeling refreshed, increasing the amount of time spent in deep sleep, increasing the quantity of peaceful dreams, and reducing the amount of sleep necessary to feel refreshed, as well as other signs of a promotion of restorative sleep as will be known to those in the art.

In embodiments, the improvement to health and wellness is any of a soothing of the body, a calming of the mind, and a reduction in physical distress; including feeling relaxed, at peace, and content; and a decrease in aches, pains, numbness, and tingling.

In embodiments, the improvement to health and wellness is an improvement of a condition or disorder linked to dysregulation or inadequate functioning of neurotransmission. In embodiments, the condition or disorder linked to dysregulation or inadequate functioning of neurotransmission is that of gabaminergic, glutaminergic, or cholinergic neurotransmission.

In embodiments, the improvement to health and wellness is an improvement in any of a pain disorder, an inflammatory disorder, and an immune or autoimmune disorder.

In embodiments, the improvement to health and wellness is an improvement in a sleep disorder.

In embodiments, the improvement to health and wellness is an improvement in a mental health disorder. In embodiments, the mental health disorder is an anxiety disorder or a substance use disorder.

In embodiments, the improvement to health and wellness includes any one or more of: a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction.

In some aspects are methods of using a vaporizer formulation including with a disclosed vaporizer device to induce a sense of euphoria, such methods comprising heating the vaporizer formulation with the vaporizer device to vaporize at least a portion of the vaporizer formulation. In some embodiments, the methods of using a vaporizer formulation or a vaporizer device result in an inducement of a sense of euphoria. In embodiments, the improvement to health and wellness is inducing a sense of euphoria.

In embodiments, a sense of euphoria includes a sense of pleasure characterized by strong feelings of happiness, excitement, and well-being. Those experiencing a sense of euphoria may describe it as being joyful and pleasurable, as well as feeling safe, secure, carefree, and supported (Cherry, What is a euphoric mood? 2022). In embodiments, a sense of euphoria is a euphoric mood. In embodiments, a sense of euphoria is a transient feeling of euphoria. A sense of euphoria may include mild euphoria as well as greater relative feelings of euphoria. A sense of euphoria may include other signs as will be known to those in the art.

In some embodiments, the A. muscaria composition provides methods of improving mental health and/or functioning, such as cognitive functioning. Improvements in mental health and functioning may include one or more of a reduction of neuroticism or psychological defensiveness, an increase in creativity or openness to experience, an increase in decision-making ability, an increase in feelings of wellness or satisfaction, or an increase in ability to fall or stay asleep. Additionally, improvements in mental health and functioning may include improvements in or a return to baseline in processing speed, learning and memory, autobiographical memory, shifting, and IQ. Measurements of such will be readily understood and appreciated according to ordinary skill. See, e.g., cognitive functioning aspects reviewed by Ahern & Semskova, Neuropsychology. 2017; 31(1): 52-72. Exemplary measures of improvements of mental health and/or functioning include the Global Assessment of Functioning (GAF) scale, the Sleep Quality Scale (SQS) and other measures of sleep quality (see, e.g., Fabbri et al., Int J Environ Res Public Health. 2021; 18(3):1082, and the Social Functioning Scale (SFS) (see, e.g., Chan et al., Psychiatry Res. 2019; 276:45-55).

In some embodiments, the disclosed A. muscaria composition may be useful as a nootropic. Nootropics or smart drugs are well-known compounds or supplements that enhance cognitive performance. They work by increasing the mental function such as memory, creativity, motivation, and attention (Suliman et al., Evidence-Based Compl, and Alt. Med. 2016; 4391375:1-12). Nootropic properties are present within the makeup of the disclosed A. muscaria formulations and, due to its cellular and molecular mechanisms of action, which enable a structural and functional plasticity, or synaptic plasticity, responsible for synaptic remodeling or known as cellular learning. In turn, nootropics mediate and enhance cognitive performance.

In some embodiments, a user of a vaporizer device in which at least a portion of the vaporizer formulation is vaporized will inhale the vaporized portion, that is, at least some quantity of the vaporized portion, such as through the mouthpiece of the device.

In certain embodiments, the disclosed A. muscaria composition is useful as an adjunct to meditation and/or meditative practice. Meditation refers to a variety of practices that focus on mind and body integration and are used to calm the mind and enhance overall well-being. While some forms of meditation involve maintaining mental focus on a particular sensation, other forms include the practice of mindfulness, which involves maintaining attention or awareness of the present moment without making judgements (see NCCIH NIH, “Meditation and Mindfulness: What You Need To Know” accessed Aug. 10, 2022). The benefits of meditation include stress reduction, decreased anxiety, decreased depression, reduction in pain (both physical and psychological), improved memory, and increased efficiency. Physiological benefits include reduced blood pressure, heart rate, lactate, cortisol, and epinephrine, decreased metabolism and breathing pattern, oxygen utilization, and carbon dioxide elimination. Neurological and physiological correlates of meditation have been researched extensively in the past, and can be measured using electroencephalogram (EEG) (Sharma H., AYU. 2015; 36:233-7).

In some embodiments, the A. muscaria composition is administered together with psychotherapy, such as psychosocial or behavioral therapy, including any of (or adapted from any of) cognitive behavioral therapy (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2): 354-59; or in Case Reports in Psychiatry, Vol. 2012, Article ID 731638), motivational interviewing based therapy (e.g., as described in J. Consul. Clin. Psychol. 2001; 69(5):858-62), or meditation based therapy, such as transcendental meditation based therapy (e.g., as described in J. Consul. Clin. Psychol. 2000; 68(3):515-52). In some embodiments, “psychotherapy” is specifically “psychedelic-assisted psychotherapy.” Psychedelic-assisted psychotherapy, broadly, includes a range of related approaches that involve at least one session where the patient ingests a psychedelic and is monitored, supported, or otherwise engaged by one or more trained mental health professionals while under the effects of the psychedelic (see, e.g., Schenberg 2018). Protocols have been developed for the standardization of procedures which emphasize a high degree of care (see, e.g., Johnson 2008), such as the therapeutic approach used by MAPS to treat patients with PTSD using MDMA (e.g., as described in Mithoefer 2017).

In some embodiments, psychotherapy comprises any accepted modality of standard psychotherapy or counseling sessions, whether once a week, twice a week, or as needed; whether in person or virtual (e.g., over telemedicine or by means of a web program or mobile app); and whether with a human therapist or a virtual or AI “therapist.” As used herein, “therapist” refers to a person who treats a patient using the formulations and methods of the invention, whether that person is a psychiatrist, clinical psychologist, clinical therapist, registered therapist, psychotherapist, or other trained clinician, counselor, facilitator, or guide, although it will be understood that certain requirements will be appropriate to certain aspects of the drug-assisted therapy (e.g., prescribing, dispensing, or administering a drug, offering psychotherapeutic support). In some embodiments, a “person” may also include an AI.

In some embodiments, a patient will participate in a treatment protocol or a method of the invention, or be administered a composition of the invention as part of such a method, if the patient meets certain specified inclusion criteria, does not meet certain specified exclusion criteria, does not meet any specified withdrawal criteria during the course of treatment, and otherwise satisfies the requirements of the embodiment of the invention as claimed.

In some embodiments, disclosed A. muscaria formulations may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention. Furthermore, a patient can participate in numerous other therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it should be understood that such participation can occur with or without the participation or guidance of a therapist.

c. Methods of Treatment

In some embodiments, disclosed formulations are used to treat a medical condition, such as a disease or disorder. In embodiments, disclosed formulations are used in the manufacture of a medicament to treat a condition, such as a disease or disorder. Also provided are methods of administering disclosed formulations to a subject having a condition, such as a disease or disorder, thereby treating said condition.

Many instances of successful treatment of a wide variety of medical conditions by self-administering Amanita muscaria are known. For an overview, see, e.g., Masha B. Microdosing with Amanita Muscaria: Creativity, Healing, and Recovery with the Sacred Mushroom. Park Street Press; 2022, which is incorporated by reference in its entirety, and which contains case studies concerning, inter alia, mental health disorders (e.g., depression, bipolar disorder, schizophrenia, substance use disorders, anxiety), sleep disorders (e.g., insomnia), CNS and neurodegenerative disorders (e.g., Parkinson's disease, Huntington's disease, autism, neuropathic pain), inflammation and inflammatory disorders, sexual dysfunctions, as well as various physical conditions (e.g., pain, hyperthyroidism, psoriasis, gastrointestinal disorders) and other indications described in embodiments herein.

In embodiments, provided are methods of treating and/or preventing a condition in a mammal, said methods comprising administering to the mammal a therapeutically effective amount of a disclosed formulation. In some embodiments, “treating” or “treatment” refers to treating a disease or disorder in a mammal, and preferably in a human, and includes causing a desired biological or pharmacological effect, such as: (a) preventing a disorder from occurring in a subject who may be predisposed to the disorder but has not yet been diagnosed with it; (b) inhibiting a disorder, i.e. arresting its development; (c) relieving a disorder, i.e., causing regression thereof; (d) protecting from or relieving a symptom or pathology caused by or related to a disorder; (e) reducing, decreasing, inhibiting, ameliorating, or preventing the onset, severity, duration, progression, frequency, or probability of one or more symptoms or pathologies associated with a disorder; and (f) preventing or inhibiting of a worsening or progression of symptoms or pathologies associated with a disorder or comorbid with a disorder. Other such measurements, benefits, and surrogate or clinical endpoints, alone or in combination, will be understood to one of ordinary skill based on the teachings herein and the knowledge in the art.

In embodiments, disclosed formulations are used to treat a central nervous system (CNS) disorder. Broadly, CNS disorders include diseases of the nervous system (e.g., movement disorders, neurodegenerative disorders) as well as mental, behavioral, and neurodevelopmental disorders, such as those characterized by the DSM-5, Merck Manual, ICD-11, or other such diagnostic resources in the art.

i. Mental, Behavioral, or Neurodevelopmental Disorders

In some embodiments, disclosed formulations are used to treat a mental, behavioral, or neurodevelopmental disorder. In embodiments, disclosed formulations are administered, such as in a therapeutically effective amount, to a subject having a mental, behavioral, or neurodevelopmental disorder, thereby treating said mental, behavioral, or neurodevelopmental disorder. In some methods herein, the disclosed formulations, when administered in a therapeutically effective amount, provide beneficial therapeutic effects for the treatment of a mental, behavioral, or neurodevelopmental disorder.

The ICD-11, which is incorporated by reference herein in its entirety, defines “mental, behavioral, or neurodevelopmental disorders” as syndromes characterized by clinically significant disturbance in an individual's cognition, emotional regulation, or behavior that reflects a dysfunction in the psychological, biological, or developmental processes that underlie mental and behavioral functioning. Such disorders include, but are not limited to, neurodevelopmental disorders, schizophrenia or other primary psychotic disorders, catatonia, mood disorders, anxiety or fear-related disorders, obsessive-compulsive or related disorders, disorders specifically associated with stress, dissociative disorders, feeding (or eating) disorders, elimination disorders, disorders of bodily distress or bodily experience, disorders due to substance use or addictive behaviors, impulse control disorders, disruptive behavior or dissocial disorders, personality disorders (and related traits), paraphilic disorders, factitious disorders, neurocognitive disorders, mental or behavioral disorders associated with pregnancy, childbirth or the puerperium, sleep-wake disorders, sexual dysfunctions, and gender incongruence.

A mental, behavioral, or neurodevelopmental disorder where otherwise undefined, will be understood to refer to the disorder as defined in the ICD-11. Within the category of mental, behavioral, or neurodevelopmental disorders, the term mental disorder (or “mental health disorder”) generally refers to a disease condition that involves negative changes in emotion, mood, thinking, and/or behavior. In general, mental health disorders are characterized by clinically significant disturbances in an individual's cognition, emotion, behavior, or a combination thereof, resulting in impaired functioning, distress, or increased risk of suffering. Although the terms “mental disorder” and “mental health disorder,” as well as terms that define specific diseases and disorders, generally shall refer to the criteria in the ICD-11, or a patient with a diagnosis based thereon, it will be appreciated that disclosed methods are equally applicable to patients having an equivalent underlying disorder, whether that disorder is diagnosed based on the criteria in ICD-11, ICD-10, DSM-5, or DSM-IV (each of which is incorporated by reference herein in its entirety) whether the diagnosis is based on other clinically acceptable criteria, or whether the patient has not yet had a formal clinical diagnosis.

In some embodiments, disclosed formulations are used to treat a mental health disorder. In embodiments, disclosed formulations are administered, such as in a therapeutically effective amount, to a subject having a mental health disorder, thereby treating said mental health disorder. In some methods herein, the disclosed formulations, when administered in a therapeutically effective amount, provide beneficial therapeutic effects for the treatment of a mental health disorder. In embodiments, disclosed formulations are used to reduce the symptoms of a mental health disorder. The symptoms of the mental health disorder to be treated shall be able to be determined by one of skill in the art, by reference to the general understanding of the art regarding that disorder.

In some embodiments, measures of therapeutic efficacy include reports by a subject or an observer. In embodiments, measures of therapeutic efficacy include responses to a questionnaire. Non-limiting representative examples of applicable measures of symptom improvement include the Generalized Anxiety Disorder Scale-7 (GAD-7), Montgomery-Asberg Depression Rating Scale (MADRS), Global Assessment of Functioning (GAF) Scale, Clinical Global Impression (CGI), Substance Abuse Questionnaire (SAQ), Mini International Neuropsychiatric Interview 5 (MINI 5), Columbia Suicide Severity Rating Scale (C—SSRS), Patient Health Questionnaire (PHQ-9), Pittsburgh Sleep Quality Index (PSQI), Interpersonal Reactivity Index (IRI), Short Form (36) Health Survey (SF-36), Self-Compassion Scale (SCS), Trauma History Questionnaire (THQ), Beck Depression Index (BDI), and related subject- or observer-reported measures.

In some embodiments, a disclosed formulation is used to treat a neurodevelopmental disorder. In embodiments, a “neurodevelopmental disorder” is a neurological and/or cognitive disorder that arises during the developmental period that involves significant difficulties in the acquisition and execution of specific neurological functions (e.g., intellectual, motor, language, or social functions). In embodiments, the neurodevelopmental disorder is a disorder of intellectual development, a developmental speech or language disorder, autism spectrum disorder, a developmental learning disorder, a developmental motor coordination disorder, attention deficit hyperactivity disorder, or stereotypic movement disorder.

In some embodiments, a disclosed formulation is used to treat schizophrenia or another primary psychotic disorder. In general, these disorders are characterized by significant impairments in reality and alterations in behavior manifest in positive symptoms like persistent delusions, persistent hallucinations, disorganized thinking and speech, grossly disorganized behavior, as well as experience of negative symptoms such as blunted or flat affect and avolition and psychomotor disturbances. In embodiments, a disclosed formulation is used to treat schizophrenia, schizoaffective disorder, schizotypal disorder, acute and transient psychotic disorder, delusional disorder, or a substance-induced psychotic disorder.

In some embodiments, a disclosed formulation is used to treat a mood disorder. As defined in the ICD-11, mood disorders are categorized according to the specific type(s) of mood episodes, and their pattern over time. The primary types of mood episodes are depressive episodes, manic episodes, mixed episodes, and hypomanic episodes. In embodiments, the mood disorder is a bipolar or related disorder (e.g., bipolar type I disorder, bipolar type II disorder, cyclothymic disorder), a depressive disorder, or a substance-induced mood disorder. In embodiments, the mood disorder is a depressive disorder. In embodiments, the depressive disorder is single-episode depressive disorder, major depressive episode disorder, persistent depressive disorder (formally known as dysthymia), disruptive mood dysregulation disorder, premenstrual dysphoric disorder, postpartum depression, substance/medication-induced depressive disorder, depressive disorder due to another medical condition, seasonal affective disorder, mixed depressive and anxiety disorder, or an unspecified depressive disorder. In embodiments, depression is assessed through the Patient Health Questionnaire-9 (PHQ-9) screening tool, Montgomery-Åsberg Depression Rating Scale (MADRS), Hamilton Depression Rating Scale, Beck Depression Inventory (BDI-II), Zung Self-Rating Depression Scales (SDS), Major Depression Inventory (MDI), Center for Epidemiologic Studies Depression Scale (CED-D), Rome Depression Inventory (RDI), Hamilton Rating Scale for Depression (HRSD), and Carroll Rating Scale (CRS).

In some embodiments, a disclosed formulation is used to treat an anxiety or fear-related disorder. An “anxiety disorder” refers to a class of mental disorders that induce excessive or abnormal fear, dread, or worry. In embodiments, the anxiety disorder is selected from the group consisting of generalized anxiety disorder, panic disorder, agoraphobia, specific phobia, social anxiety disorder, separation anxiety disorder, selective mutism, or a substance-induced anxiety disorder.

In some embodiments, a disclosed formulation is used to treat an obsessive-compulsive or related disorder. In general, these disorders are characterized by repetitive thoughts and behaviors, such as cognitive phenomena (obsessions, intrusive thoughts and preoccupations). In embodiments, the disorder is characterized by a compulsive need to accumulate possessions and distress related to discarding them (i.e., hoarding disorder). In embodiments, the disorder is body-focused and can be characterized by recurrent and habitual actions (hair-pulling, skin-picking). In embodiments, the disorder is obsessive-compulsive disorder, body dysmorphic disorder, olfactory reference disorder, hypochondriasis, hoarding disorder, a body-focused repetitive behavior disorder, or a substance-induced obsessive-compulsive disorder.

In some embodiments, a disclosed formulation is used to treat a disorder associated with stress. In embodiments, the disorder associated with stress has an identifiable stressor that is a causal factor, like exposure to a stressful or traumatic event, or a series of such events or adverse experiences. Stressors may be within the normal range of life experiences (e.g., divorce, socioeconomic problems), or from a threatening or traumatizing experience. In general, the nature and duration of the symptoms that arise in response to the stressor can distinguish the disorder from everyday stress. In embodiments, a disclosed formulation is used to treat post-traumatic stress disorder, complex post-traumatic stress disorder, prolonged grief disorder, adjustment disorder, reactive attachment disorder, or disinhibited social engagement disorder.

In some embodiments, a disclosed formulation is used to treat a feeding or eating disorder. Feeding or eating disorders generally involve abnormal eating or feeding behaviors that are not explained by another health condition, and are not developmentally appropriate or culturally sanctioned. These disorders can involve preoccupation with food as well as body weight and shape concerns. In embodiments, a disclosed formulation is used to treat anorexia nervosa (including anorexia with significantly low body weight, anorexia with dangerously low body weight, or anorexia in recovery with normal body weight), bulimia nervosa, binge eating disorder, avoidant-restrictive food intake disorder, pica, or rumination-regurgitation disorder.

In some embodiments, a disclosed formulation is used to treat a disorder of bodily distress or bodily experience. Disorders of bodily stress typically involve bodily symptoms that the subject finds distressing and to which the subject devotes excessive attention. Bodily integrity dysphoria typically involves a disturbance in the person's experience of the body manifested by persistent discomfort or intense feelings of body configuration. In embodiments, a disclosed formulation is used to treat a bodily distress disorder (including mild, moderate, and severe bodily distress disorder) or body integrity dysphoria.

In some embodiments, a disclosed formulation is used to treat a disorder due to substance use or addictive behaviors. Individual self-administration of Amanita muscaria mushrooms is reportedly effective for treating various addictions, including substance addiction (e.g., nicotine addiction, alcohol addiction) as well as behavioral addictions (e.g., pornography addiction, other compulsive behaviors). (See, e.g., Masha B. Microdosing with Amanita Muscaria: Creativity, Healing, and Recovery with the Sacred Mushroom. Park Street Press; 2022.) Disorders due to substance use or addictive behaviors are mental and/or behavioral disorders that develop predominantly as a result of the use of psychoactive substances (including medications and illegal or illicit substances), or specific repetitive rewarding and reinforcing behaviors. In embodiments, a disclosed formulation is used to treat disorders due to substance use (i.e., a substance use disorder, or SUD). In embodiments, the substance use disorder is associated with alcohol, cannabis, synthetic cannabinoids, opioids, sedatives, hypnotics or anxiolytics, cocaine, stimulants (e.g., amphetamines, methamphetamines, methcathinone, synthetic cathinones, caffeine), hallucinogens, nicotine, volatile inhalants, MDMA or MDA, dissociative drugs like ketamine and phencyclidine, or another substance (including medications and non-psychoactive substances). In embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In embodiments, the substance use disorder is alcohol use disorder. In embodiments, the substance use disorder is cannabis use disorder. In embodiments, the substance use disorder is caffeine use disorder. In embodiments, the substance use disorder is phencyclidine use disorder. In embodiments, the substance use disorder is inhalant use disorder. In embodiments, the substance use disorder is opioids use disorder. In embodiments, the substance use disorder is sedatives use disorder. In embodiments, the substance use disorder is hypnotics use disorder. In embodiments, the substance use disorder is anxiolytics use disorder. In embodiments, the substance use disorder is stimulants use disorder. In embodiments, the substance use disorder is tobacco use disorder. In embodiments, the substance use disorder is alcohol use disorder, wherein said alcohol use disorder is selected from alcohol abuse, alcohol dependence, and alcoholism. In embodiments, the disorder is associated with another addictive behavior (e.g., gambling disorders, gaming disorder). In embodiments, a substance use disorder can be screened using a Screening to Brief Intervention (S2BI), Alcohol, Smoking, and Substance Involvement Screening Test (ASSIST), Brief Screener for Alcohol, Tobacco, and other Drugs (BSTAD), Tobacco, Alcohol, Prescription medication, and other Substance use (TAPS), the Opioid Risk Tool—OUD (ORT-OUD) Chart, Drug Abuse Screen Test (DAST-10), and Tobacco, Alcohol, Prescription medication, and other Substance use (TAPS).

In some embodiments, the behavioral addiction is gambling disorder. In embodiments, the behavioral addiction is gaming disorder. In embodiments, the behavioral addiction is sexual addiction. In embodiments, the behavioral addiction is compulsive buying disorder. In embodiments, the behavioral addiction is technology addiction. In embodiments, the behavioral addiction is pornography addiction.

In some embodiments, the disclosed A. muscaria formulations reduce compulsive behaviors. Compulsive behaviors are described, e.g., in the ICD-11.

In some embodiments, a disclosed formulation is used to treat an impulse control disorder. In general, impulse control disorders are characterized by the repeated failure to resist an impulse, drive, or urge to perform an act that is rewarding to the subject despite negative long-term consequences, such as harm to the subject or a significant impairment in important areas of the subject's functioning. In embodiments, impulse control behaviors include fire-setting, stealing, inappropriate sexual behavior, and explosive outbursts. In embodiments, a disclosed formulation is used to treat pyromania, kleptomania, compulsive sexual behavior disorder, or intermittent explosive disorder.

In some embodiments, a disclosed formulation is used to treat a personality disorder. Personality disorders may be generally characterized by problems in perceiving one's identity, self-worth, accuracy of self-view, and self-discretion that is manifest in patterns of cognition, emotional experience, emotional expression, and maladaptive behavior. In embodiments, a disclosed formulation is used to treat a mild, moderate, or severe personality disorders. In embodiments, a disclosed formulation is used to treat a prominent personality trait or patterns (e.g., negative affectivity, detachment, disociability, disinhibition, anankastia, borderline pattern). In embodiments, the personality disorder is antisocial personality disorder, avoidant personality disorder, borderline personality disorder, dependent personality disorder, histrionic personality disorder, masochistic or sadistic behavior, narcissistic personality disorder, obsessive-compulsive personality disorder, paranoid personality disorder, psychopathy, sociopathy, schizoid personality disorder, or schizotypal personality disorder.

In some embodiments, a disclosed formulation is used to treat a paraphilic disorder. Paraphilic disorders can be characterized by persistent and intense patterns of atypical sexual arousal, the focus of which involves others whose age or status renders them unwilling or unable to consent. In embodiments, a disclosed formulation is used to treat exhibitionistic disorder, voyeuristic disorder, pedophilic disorder, coercive sexual sadism disorder, frotteuristic disorder, other paraphilic disorders involving non-consenting individuals, or paraphilic disorders involving solitary behavior or consenting individuals.

In some embodiments, a disclosed formulation is used to treat a neurocognitive disorder. Neurocognitive disorders may be characterized by primary clinical defects in cognitive functioning that are acquired (rather than developmental), and therefore the subject experiences a decline from a previously attained level of functioning. In embodiments, a disclosed formulation is used to treat delirium. In embodiments, the delirium is associated with another disease or disorder. In embodiments, the delirium is associated with a psychoactive substance (including medications and illicit or illegal substances). In embodiments, a disclosed formulation is used to treat mild neurocognitive disorder. In embodiments, a disclosed formulation is used to treat an amnestic disorder. In embodiments, the amnestic disorder is associated with another disease or disorder. In embodiments, the delirium is associated with a psychoactive substance (including medications and illicit or illegal substances). In embodiments, a disclosed formulation is used to treat dementia. In embodiments, the dementia is associated with Alzheimer's disease, Parkinson's disease, cerebrovascular disease, Lewy body disease, a psychoactive substance (including medications and illicit or illegal substances). In embodiments, a disclosed formulation is used to treat a behavioral or psychological disturbance associated with dementia. In embodiments, dementia is assessed using a Functional Activities Questionnaire (FAQ), Ascertain Dementia 8 (AD8), Mini-Cog, Mini-Mental State Exam (MMSE), the Montreal Cognitive Assessment (MoCA), and the Neuropsychiatric Inventory Questionnaire (NPI-Q).

In some embodiments, a disclosed formulation or composition is administered together with psychotherapy, such as psychosocial or behavioral therapy, including any of (or adapted from any of) cognitive behavioral therapy (CBT) (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2):354-59; or in Case Reports in Psychiatry, Vol. 2012, Article ID 731638), motivational interviewing based therapy (e.g., as described in J. Consul. Clin. Psychol. 2001; 69 (5): 858-62), meditation based therapy, such as transcendental meditation based therapy (e.g., as described in J. Consul. Clin. Psychol. 2000; 68(3):515-52), or the therapeutic approach used by MAPS to treat patients with PTSD (e.g., as described in Mithoefer, M (2017). A Manual for MDMA-Assisted Psychotherapy in the Treatment of Post-traumatic Stress Disorder).

In some embodiments, disclosed formulations may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention. Furthermore, a patient can participate in numerous other therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it should be understood that such participation can occur with or without the participation or guidance of a therapist.

ii. CNS Disorders and Neurodegenerative Conditions

The GABAergic system is implicated in neurodegeneration; for example, imbalance between glutamate and GABA has been shown to decrease synaptic strength and hence lead to neurodegeneration, which may in turn contribute to the progression of neurodegenerative diseases like Alzheimer's disease, Parkinson's disease, Huntington's disease, and multiple sclerosis (Sood et al. JNR. 2021; 99 (12): 3148-3189). In embodiments, the disclosed A. muscaria formulations are used to treat a CNS disorder or neurodegenerative condition. In embodiments, the disclosed A. muscaria formulations are used in the manufacture of a medicament to treat a CNS disorder or neurodegenerative condition. In embodiments, the disclosed A. muscaria formulations, e.g., in a therapeutically effective amount, are administered to a subject having a CNS disorder or neurodegenerative condition to treat said CNS disorder or neurodegenerative condition. In embodiments, a disclosed A. muscaria composition prevents or treats, such as alleviates, a CNS disorder or neurodegenerative condition mediated by viral infection.

Neurodegeneration may be assessed, e.g., by measuring markers of neuronal loss, such as cerebrospinal fluid markers, e.g., visinin-like protein 1 (VILIP-1), tau, and p-tau181 (Tarawneh et al., Neurol. 2015; 72(6): 656-665). Cognitive decline may also be used as a measure of neurodegeneration. Methods for assessing cognitive decline, e.g., comprehensive neuropsychological testing, are known to one of skill in the art. Exemplary cognitive evaluations include Mini-Mental State Examination (MMSE) and Montreal Cognitive Assessment (MoCA). See, e.g., Toh et al., Transl Neurodegener. 2014; 3:15. Cognitive decline and the progression of disease state may also be assessed using a condition-specific measure, e.g., the Unified Huntington's Disease Rating Scale (UHDRS).

Neurodegenerative conditions, such as diseases or disorders include, e.g., dementia, Alzheimer's disease, Huntington's disease, multiple sclerosis, and Parkinson's disease. A feature of neurodegenerative conditions is neuronal cell death, which, among other aspects, has been implicated in the promotion of inflammation. See, e.g., Chan et al., Annu Rev Immunol. 2015; 33:79-106 and Chi et al., Int J Mol Sci. 2018; 19 (10): 3082. Neurodegenerative diseases can be classified according to primary clinical features, e.g., dementia, parkinsonism, or motor neuron disease, anatomic distribution of neurodegeneration, e.g., frontotemporal degenerations, extrapyramidal disorders, or spinocerebellar degenerations, or principal molecular abnormality (Dugger & Dickson, Cold Spring Harb Perspect Biol. 2017; 9(7):a028035.

iii. Pain and Pain Disorders

In general, GABA agonists have known utility in treating pain and pain disorders (Enna & McCarson. Adv. Pharmacol. 2006; 54:1-27). Muscimol specifically has also shown promise for this purpose. (See, e.g., Hosseini et al. Emerg (Tehran). 2014 Autumn; 2(4):151-157 and Laviola & Alleva. Psychopharmacology 1990; 102:41-48.) In some embodiments, disclosed formulations are used to treat a pain disorder. In embodiments, disclosed formulations are administered, such as in a therapeutically effective amount, to a subject having a pain disorder, thereby treating said pain disorder. In some methods herein, the disclosed formulations, when administered in a therapeutically effective amount, provide beneficial therapeutic effects for the treatment of a pain disorder.

A “pain disorder” refers to a class of medical conditions characterized by the experience of persistent or recurrent physical or psychological pain, either localized or widespread, that significantly impairs an individual's daily functioning and quality of life. These disorders may involve various etiologies, including but not limited to nociceptive, neuropathic, psychogenic, idiopathic or radicular origins. In embodiments, a compound is used to treat neuropathic pain. In some embodiments, a compound is used to treat psychogenic pain. In embodiments, a compound is used to treat idiopathic pain. In embodiments, a compound is used to treat radicular pain.

Pain disorders may manifest as acute or chronic pain, and they can affect different parts of the body, such as musculoskeletal, neurological, gastrointestinal, or visceral systems. Pain can be expressed as including but not limited to, post-herpetic pain, trigeminal pain, occipital pain, or pudendal pain. In some embodiments, a disclosed formulation is used to treat pain associated with cancer or chemotherapy. In embodiments, a disclosed formulation is used to treat arthritis, back pain, central pain, chronic fatigue syndrome, cluster headaches, migraine headaches, complex regional pain syndrome, compression mononeuropathy, diabetic neuropathy, fibromyalgia, focal neuropathy, herniated disc pain, or sciatica.

In some embodiments, pain is assessed using the Pain, Enjoyment, and General Activity Scale (PEG), the Numeric Rating Scale (NRS), the Visual Analog Scale (VAS), Behavioral Pain Scale (BPS), and the Faces Pain Scale-Revised (FPS-R).

iv. Inflammation and Inflammatory Disorders

It has been reported that muscimol inhibits inflammation, potentially through GABA agonism and other mechanisms (Li et al. Can. J. Physiol. Pharmacol. 2022; 100 (7): 665-678). Furthermore, the GABAergic system is known to play a role in the inflammatory response (Barragan et al. Acta. Physiol. 2015; 213(4):819-827). In some embodiments, the disclosed A. muscaria formulations are used to treat inflammation or an inflammatory disorder. In embodiments, the disclosed A. muscaria formulations are used in the manufacture of a medicament to treat an inflammatory disorder. In embodiments, the disclosed A. muscaria formulations, e.g., in a therapeutically effective amount, are administered to a subject having an inflammatory disorder to treat said pain disorder and/or said inflammatory disorder.

In some embodiments, an inflammatory disorder is a disorder that causes acute inflammation, or that exhibits chronic inflammation as a symptom, including any of pressure ulcers, including acne vulgaris; oxalic acid/heartburn, age-related macular degeneration (AMD), allergies, allergic rhinitis, Alzheimer's disease, amyotrophic lateral sclerosis, Anemia, appendicitis, arteritis, arthritis, including osteoarthritis, rheumatoid arthritis, juvenile idiopathic arthritis, spondyloarthropathy such as ankylosing spondylitis, reactive arthritis (Reiter syndrome), psoriatic arthritis, enteroarthritis associated with inflammatory bowel disease, Whipple and Behcet's disease, septic arthritis, gout (also known as gouty arthritis, crystalline synovitis, metabolic arthritis), pseudogout (calcium pyrophosphate deposition disease), and Still's disease. Arthritis can affect a single joint (monoarthritis), two to four joints (oligoarthritis), or five or more joints (polyarthritis); asthma, atherosclerosis, autoimmune disorder, balanitis, blepharitis, bronchiolitis, bronchitis, bullous pemphigoid, burns, bursitis, cancer, including NF-κB-induced inflammatory cancer; cardiovascular disease, including hypertension, endocarditis, myocarditis, heart valve dysfunction, congestive heart failure, myocardial infarction, diabetic heart abnormalities, vascular inflammation, including arteritis, phlebitis, and vasculitis; arterial occlusive disease, including arteriosclerosis and stenosis; inflammatory cardiac hypertrophy, peripheral arterial disease, aneurysm, embolism, incision, pseudoaneurysm, vascular malformation, vascular nevus, thrombosis, thrombophlebitis, varicose veins, stroke, cardiac arrest, and carditis; celiac disease, cellulitis, cervicitis, cholangitis, cholecystitis, chorioamnionitis, chronic obstructive pulmonary disease (COPD), cirrhosis, congestive heart failure, conjunctivitis, colitis, cyclophosphamide-induced cystitis, cystic fibrosis, cystitis, cold, lacrimal inflammation, dementia, dermatitis, including atopic dermatitis, chronic photosensitivity dermatitis, eczema, atopic eczema, contact eczema, dryness eczema, seborrheic eczema, sweating disorders, discoid eczema, venous eczema, herpetic dermatitis, neurodermatitis, and autosensitising dermatitis, stasis dermatitis, purulent sweaty, lichen planus, psoriasis, including psoriasis vulgaris, nail psoriasis, prickly psoriasis, scalp psoriasis, inverse psoriasis, pustular psoriasis, erythrodermic psoriasis, and psoriatic arthritis; rosacea, and scleroderma, including morphea; pharmacologically induced inflammation, including from legal or illegal drugs, and chemicals; chronic neurogenic inflammation, including primary and secondary neural inflammation; dermatomyositis, diabetes, diabetic neuropathy, diabetic retinopathy, diabetic nephropathy, diabetic ulcer, digestive system disease, emphysema, encephalitis, endocarditis, endometritis, enterocolitis, epicondylitis, epididymis, fasciitis, fibromyalgia, fibrosis, connectitis, gastritis, gastroenteritis, gingivitis, glomerulonephritis, glossitis, heart disease, heart Valvular dysfunction, hepatitis, purulent spondylitis, Huntington's disease, hyperlipidemic pancreatitis, hypertension, ileitis, infection, including lymphangitis, lymphadenitis, bacterial cystitis, bacterial encephalitis, pandemic influenza, viral encephalitis, and viral hepatitis (types A, B, and C); inflammatory bowel disease, including Crohn's disease; inflammatory heart enlargement, inflammatory neuropathy, insulin resistance, between Interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), migraine, multiple sclerosis, myelitis, myocarditis, myositis, nephritis, non-alcoholic steatohepatitis, obesity, umbilitis, ovitis, testitis, osteochondritis, osteopenia, osteomyelitis, osteoporosis, osteomyelitis, otitis, pancreatitis, Parkinson's disease, parotitis, pelvic inflammatory disease, pemphigus vulgaris, pericarditis, Peritonitis, pharyngitis, phlebitis, pleurisy, interstitial pneumonia, polycystic nephritis, polymyositis, proctitis, prostatitis, psoriasis, pulpitis, pyelonephritis, portal vein, renal failure, reperfusion injury, retinitis, rheumatic fever Rhinitis, fallopianitis, sarcoidosis, salivary glanditis, sepsis, including bacteremia and viremia; sinusitis, spastic colon, stenosis, stomatitis, stroke, surgical complications, synovitis, tendonitis, tendonitis, tendonitis, thrombophlebitis, tonsillitis, trauma, traumatic brain injury, graft rejection, including graft versus host disease (GVHD); a Th1-mediated inflammatory disease, trigonitis, tuberculosis, tumor, urethritis, bursitis, uveitis, vaginitis, vasculitis, including Buerger's disease, cerebral vasculitis, Churg-Strauss arteritis, cryoglobulinemia, essential cryoglobulin vasculitis, giant cells arteritis, golfer vasculitis, Henoch-Schonlein purpura, hypersensitivity vasculitis, Kawasaki disease, microscopic polyarteritis/polyvasculitis, nodular polyarteritis, rheumatoid polymuscular muscle pain (PMR), rheumatic vasculitis, Takayasu arteritis, Wegener's granulomatosis, systemic lupus erythematosus (SLE), relapsing polychondritis, Behcet's disease; ulcerative colitis such as ulcerative proctitis, left side colitis, total colitis, and fulminant colitis; and vulvitis.

In embodiments, chronic inflammation includes tissue inflammation, e.g., skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation intestinal inflammation, neuroinflammation, and brain inflammation.

In some embodiments, the disclosed A. muscaria formulations are used to reduce inflammation. In embodiments, the disclosed A. muscaria formulations are used in the manufacture of a medicament to reduce inflammation. In embodiments, the disclosed A. muscaria formulations, e.g., in a therapeutically effective amount, are administered to a subject to reduce inflammation.

A reduction in inflammation, such as chronic systemic inflammation, may be measured according to various methods available to one of skill. Inflammatory biomarkers may be detected from biological specimens, for example, a subject's blood, such as plasma or serum, or saliva. In one example, inflammation may be detected by measuring high-sensitivity C-reactive protein (CRP) and white blood cell count from a blood test. CRP may also be detected in a saliva sample. Salivary CRP is not synthesized locally in the mouth and may reflect more systemic levels of inflammation compared to other inflammatory biomarkers, such as cytokines (Szabo & Slavish, Psychoneuroendocrinology. 202; 124:105069). Additionally clinical pathology data, e.g., hematology data on erythrocyte parameters, platelet count, total number of leukocytes, and leukocyte differentials and morphology, coagulation data on clotting times and fibrinogen, and clinical chemistry data on total protein, albumin and globulin, liver enzymes, renal parameters, electrolytes, and bilirubin can provide an initial indication of the presence and potentially the location of inflammation, in the absence of specific data on immune tissues. See, e.g., Germolec et al., Methods Mol Biol. 2018; 1803:57-79 and Luo et al., Clin Lab. 2019 1; 65(3).

### V. Sleep Disorders

GABA agonists have been reported to modulate sleep (Harrison, N. J. Clin. Psychiatry 2007; 68(suppl 5): 6-12) and hence may be useful in the treatment of sleep disorders. In some embodiments, the disclosed A. muscaria formulations are used to treat a sleep disorder. In embodiments, the disclosed A. muscaria formulations are used in the manufacture of a medicament to treat a sleep disorder. In embodiments, the disclosed A. muscaria formulations, e.g., in a therapeutically effective amount, are administered to a subject having a sleep disorder to treat said sleep disorder. In embodiments are methods of treating health conditions, wherein the health condition is a sleep disorder. In embodiments, the sleep disorder may or may not be comorbid with one or more mental health disorders.

The Diagnostic Classification of Sleep and Arousal Disorders (DSCAD) classifies sleep disorders broadly into nine categories: (1) psychophysiological insomnia; (2) sleep disorders associated with mental disorders; (3) sleep disorders associated with a regular use of drugs and alcohol; (4) insomnias associated with sleep-induced breathing disorders; (5) sleep disorders associated with nocturnal myoclonus and restless legs syndrome (RLS); (6) sleep disorders by other disorders, drugs, and environmental conditions; (7) childhood onset insomnias; (8) other types of insomnia; and (9) sleep abnormalities with no symptoms of insomnia. Each of these categories is based on the state of disorder.

The International Classification of Sleep Disorders (ICSD) classifies sleep disorders broadly into four categories: (1) dyssomnias comprising disorders that are primarily disorders of sleep per se [for example, intrinsic sleep disorders such as narcolepsy, extrinsic sleep disorders, and circadian rhythm sleep disorders]; (2) parasomnias comprising disorders of abnormal behaviors that occur during sleep (also known as abnormal behavior during sleep) [arousal disorders, sleep-wake transition disorders, parasomnias usually associated with REM sleep, and other parasomnias]; (3) sleep disorders associated with medical/psychiatric disorders [sleep disorders associated with mental disorders, sleep disorders associated with neurologic disorders, and sleep disorders associated with other medical disorders]; and (4) proposed sleep disorders, for example, short sleeper, long sleeper, subwakefulness syndrome, and the like. To this date, this classification includes about 90 categories of sleep disorders. Currently, further classifications have been made continuously based on etiology.

According to the International Classification of Diseases, Tenth Edition (ICD-10) (1992), which have been published by World Health Organization (WHO), sleep disorders are classified into (1) nonorganic sleep disorders (F51: for example, nonorganic insomnia, nonorganic hypersomnia, sleep walking, sleep terrors, nonorganic disorder of the sleep-wake schedule, nightmares, and the like); (2) sleep disorders [G47: for example, sleep apnoea, disorders of initiating and maintaining sleep (insomnias), disorders of excessive somnolence (hypersomnia), cataplexy, narcolepsy, cataplexy attacks, disorders of the sleep-wake schedule (such as irregular sleep pattern, sleep rhythm disorder, and delayed sleep phase syndrome), and the like]; (3) other respiratory conditions originating in the perinatal period (P28: for example, primary sleep apnoea of newborn, and the like); and (4) personal history of risk-factors, not elsewhere classified (Z91: e.g., personal history of unhealthy sleep-wake schedule, and the like).

The Merck Manual, 17th ed., Section 14, Ch. 173 defines sleep disorders as disorders that affect the ability to fall asleep, stay asleep, or stay awake or that produce sleep-related abnormal behaviors, and the like. According to the Manual, sleep disorders are classified into the following symptoms: (1) insomnia (disorders falling asleep, difficulty staying asleep, or a disturbance in sleep patterns that causes inadequate sleep, and the like; for example, sleep-onset insomnia (difficulty falling asleep), early morning awakening, sleep-wake reversals, rebound insomnia, and the like); (2) hypersomnia (defined as a pathological increase of at least 25% in total sleeping time; for example, narcolepsy, sudden episode of sleep, and the like); (3) sleep apnoea syndromes, parasomnias (based on patients' chief complaints); and the like.

Herein, when referring to sleep disorders, one will understand the term to encompass, unless otherwise defined, any of the above definitions (including as since revised) and such other generally accepted definitions known to those in the art. In clinical treatment, sleep disorders are often broadly classified into (1) insomnias, (2) hypersomnia, (3) parasomnias, and (4) disorders of sleep-wake schedule (corresponding to the circadian rhythm sleep disorders of ICSD), made symptomatically based on patients' chief complaints, separately from the international classifications described above. Depending on etiology, a different medical treatment is given to each type of sleep disorder. In some embodiments, the sleep disorder is any of an insomnia, a hypersomnia, a parasomnia, and a disorder of sleep-wake schedule.

In some embodiments, the disclosed A. muscaria formulations are used to treat sleep disorders such as, but not limited to, insomnia disorders, sleep-related breathing disorders, central disorders of hypersomnolence, circadian rhythm sleep-wake disorders, sleep-related movement disorders, parasomnias, non-24-hour sleep wake disorder, excessive daytime sleepiness, shift work disorder, and others. In embodiments, the disclosed A. muscaria formulations are used in the manufacture of a medicament to treat sleep disorders such as, but not limited to, insomnia disorders, sleep-related breathing disorders, central disorders of hypersomnolence, circadian rhythm sleep-wake disorders, sleep-related movement disorders, parasomnias, non-24-hour sleep wake disorder, excessive daytime sleepiness, shift work disorder, and others. See, e.g., Sateia, Chest. 2014; 146(5):1387-1394. Regular sleep and sleep quality is associated with enhanced longevity (Dew et al., Psychosomatic Medicine, 2003; 65(1): 63-73; Kripke et al., Arch Gen Psychiatry. 1979; 36(1): 103-16; Mazzotti et al., Front Aging Neurosci. 2014; 6:134).

In some embodiments, the sleep disorder is insomnia. In embodiments, the disclosed A. muscaria formulations are used to treat insomnia. In embodiments, the disclosed A. muscaria formulations are used in the manufacture of a medicament to treat insomnia. Broadly, insomnia is defined as a persistent difficulty with sleep initiation, duration, consolidation, or quality, by the ICSD-3 manual. Chronic insomnia is characterized by persistent insomnia that occurs at least three times per week for at least three months, while the same symptoms for less than three months is termed short-term insomnia.

Regardless of whether an individual is diagnosed with chronic, or short-term insomnia, most instances of the disorder present as either sleep-onset insomnia, or sleep maintenance insomnia, wherein the former is characterized by a difficulty falling asleep, while those suffering with the latter experience trouble staying asleep. Criteria for diagnosing insomnia include (1) a report of sleep initiation or maintenance problems, (2) adequate opportunity and circumstances to sleep, and (3) daytime consequences (Sateia, Chest. 2014; 146(5):1387-1394).

There are multiple factors that may cause, or exasperate symptoms of insomnia; however, stress and anxiety are each significant contributing factors. The anxiolytic effects of the disclosed A. muscaria formulations, including the promotion of calmness and reduction of stress, are effective in treating insomnia, including chronic insomnia and short-term insomnia characterized as either sleep-onset or sleep maintenance insomnia, by treating some of its underlying causes.

In some embodiments, a disclosed formulation is used to treat a sleep-wake disorder. In general, sleep-wake disorders are associated with difficulty initiating or maintaining sleep (e.g., insomnia), excessive sleepiness (e.g., hypersomnolence disorders), respiratory disturbance during sleep (e.g., sleep-related breathing disorders (SRBDs), such as obstructive sleep apnea (OSA), central sleep apnea (CSA), sleep-related hypoventilation disorders, sleep-related hypoxemia disorder, snoring, cataphrenia, Cheyne-Stokes breathing, and sleep-disordered breathing), disorders of the sleep-wake schedule (e.g., circadian rhythm sleep-wake disorders), abnormal movements during sleep, or problematic behavioral or psychological events that occur while falling asleep, during sleep, or upon arousal from sleep (e.g., parasomnia disorders). In embodiments, a disclosed formulation is used to treat an insomnia disorder, a hypersomnolence disorder, a sleep-related breathing disorder, a circadian rhythm sleep-wake disorder, or a parasomnia disorder.

In some embodiments, the disclosed A. muscaria formulations are used to treat a circadian rhythm sleep-wake disorder. In embodiments, the disclosed A. muscaria formulations are used in the manufacture of a medicament to treat a circadian rhythm sleep-wake disorder. Delayed sleep-wake phase disorder, advanced sleep-wake phase disorder, irregular sleep-wake rhythm disorder, non-24-h sleep-wake rhythm disorder, shift work disorder, and jet lag disorder are all classified as circadian rhythm sleep-wake disorders. Such disorders are diagnosed with the following criteria: (1) a chronic or recurrent pattern of sleep-wake rhythm disruption primarily caused by an alteration in the endogenous circadian timing system or misalignment between the endogenous circadian rhythm and the sleep-wake schedule desired or required, (2) a sleep-wake disturbance (ie, insomnia or excessive sleepiness, and (3) associated distress or impairment (Sateia, Chest. 2014; 146(5):1387-1394)

In one example, Non-24-Hour Sleep Wake disorder (N24SWD) refers to a condition wherein an individual's natural circadian rhythm is shorter or (more commonly) longer than 24 hours (Pacheco, Sleep Foundation, “Non-24-Hour Sleep Wake Disorder,” 2022). This causes such individuals to struggle with fluctuations in appetite, mood, and alertness that—while following their body's internal clock—is generally at odds with rising and setting of the sun. When heavily desynchronized, individuals with N24SWD exhibit a natural preference for sleeping during the day, and experience difficulty sleeping at night.

In some embodiments, the disclosed A. muscaria formulations are used to treat shift work disorder. In embodiments, the disclosed A. muscaria formulations are used in the manufacture of a medicament to treat shift work disorder. Shift work disorder is generally caused by an activity (e.g., an obligatory obligation like work, as the name suggests) that forces an individual to stay awake when they would ordinarily be asleep. This causes individuals to lose, on average, between 1-4 hours of sleep each day, leading to lethargy, mood swings, low testosterone, and negatively impacting the ability to function while attempting to accomplish day-to-day tasks (Pacheco, Sleep Foundation, “Diagnosing Shift Work Disorder,” 2022).

In some embodiments, the disclosed A. muscaria formulations are used to treat excessive daytime sleepiness. In embodiments, the disclosed A. muscaria formulations are used in the manufacture of a medicament to treat excessive daytime sleepiness. As the name implies, excessive daytime sleepiness is characterized by abnormal levels of drowsiness during the day, and a desire to sleep (Pacheco, American Sleep Association, “Excessive Daytime Sleepiness: Causes, Test and Treatments,” 2022). Excessive daytime sleepiness may be caused by a combination of complex factors, but is generally due to a chronic lack of sleep, or fragmented, poor-quality sleep.

Poor sleep quality is known to contribute to a wide range of diseases, including mental health conditions, neurological disorders, neurodegenerative diseases, and cardiovascular diseases. (See, e.g., Tobaldini et al. Neurosci. Biobehav. Rev. 2017; 74(B): 321-329; Raggi & Ferri. Eur. Neurol. 2010; 17(11):1326-1338; Palagini et al. J. Sleep Res. 2022; 31(4):e13628; and Anderson & Bradley. Nat. Sci. Sleep. 2013; 5:61-75). Hence, in some embodiments, disclosed formulations are useful in the treatment of such conditions (which are described in further detail in various embodiments herein) through a mechanism in which improving sleep quality in an individual, whether or not said individual has been diagnosed with a sleep disorder, treats a concomitant medical condition that is associated with poor sleep quality. In embodiments, improving sleep quality in an individual through the use of disclosed vaporizer formulations is useful in the treatment of asthma, diabetes, heart attack, stroke, or cancer.

vi. Immune Disorders

The GABAergic system is implicated in immune cell functions, inflammatory conditions and diseases in peripheral tissues, and hence GABA agonists may be promising drugs for a variety of immune-mediated medical conditions (Barragan et al. Acta. Physiol. 2015; 213(4):819-827). In some embodiments, the disclosed A. muscaria formulations are used to treat an immune disorder. In embodiments, the disclosed A. muscaria formulations are used in the manufacture of a medicament to treat an immune disorder. In embodiments, the disclosed A. muscaria formulations, e.g., in a therapeutically effective amount, are administered to a subject having an immune disorder to treat said immune disorder. Autoimmune diseases can be divided into systemic and organ-specific autoimmune disorders according to the main clinical and pathological characteristics of each disease.

In some embodiments, immune disorders include disorders characterized by deregulation of Toll-like receptor signaling and/or type I interferon-mediated immunity. In embodiments, the immune disorder is any of acne vulgaris, acute respiratory distress syndrome, Addison's disease, adrenocortical insufficiency, adrenogenital syndrome, allergic conjunctivitis, allergic rhinitis, allergic intraocular inflammatory diseases, ANCA-associated small-vessel vasculitis, angioedema, ankylosing spondylitis, aphthous stomatitis, arthritis, asthma, atherosclerosis, atopic dermatitis, Behcet's disease, Bell's palsy, berylliosis, bronchial asthma, bullous herpetiformis dermatitis, bullous pemphigoid, carditis, celiac disease, cerebral ischaemia, chronic obstructive pulmonary disease, cirrhosis, Cogan's syndrome, contact dermatitis, COPD, Crohn's disease, Cushing's syndrome, dermatomyositis, diabetes mellitus, discoid lupus erythematosus, eosinophilic fasciitis, epicondylitis, erythema nodosum, exfoliative dermatitis, fibromyalgia, focal glomerulosclerosis, giant cell arteritis, gout, gouty arthritis, graft-versus-host disease, hand eczema, Henoch-Schonlein purpura, herpes gestationis, hirsutism, hypersensitivity drug reactions, idiopathic cerato-scleritis, idiopathic pulmonary fibrosis, idiopathic thrombocytopenia purpura, inflammatory bowel or gastrointestinal disorders, inflammatory dermatoses, juvenile rheumatoid arthritis, laryngeal edema, lichen planus, Loeffler's syndrome, lupus nephritis, lupus vulgaris, lymphomatous tracheobronchitis, macular edema, multiple sclerosis, musculoskeletal and connective tissue disorder, myasthenia gravis, myositis, obstructive pulmonary disease, ocular inflammation, organ transplant rejection, osteoarthritis, pancreatitis, pemphigoid gestationis, pemphigus vulgaris, polyarteritis nodosa, polymyalgia rheumatica, primary adrenocortical insufficiency, primary biliary cirrhosis, pruritus scroti, pruritis/inflammation, psoriasis, psoriatic arthritis, Reiter's disease, relapsing polychondritis, rheumatic carditis, rheumatic fever, rheumatoid arthritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, serum sickness, shoulder tendinitis or bursitis, Sjogren's syndrome, Still's disease, stroke-induced brain cell death, Sweet's disease, systemic dermatomyositis, systemic lupus erythematosus, systemic sclerosis, Takayasu's arteritis, temporal arteritis, thyroiditis, toxic epidermal necrolysis, type-1 diabetes, ulcerative colitis, uveitis, vasculitis, and Wegener's granulomatosis.

In some embodiments, the immune disorder is an autoimmune disorder. In embodiments, an autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune disease, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopenia purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, and systemic lupus erythematosus; morphea, multiple sclerosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo.

vii. Sexual Dysfunction

Rodent models have shown that GABAergic drugs can modulate sex drive. (See, e.g., Agmo & Paredes. Eur. J. Pharmacol. 1985; 112(3):371-378 and Bitran et al. Pharmacol. Biochem. Behav. 1988; 31(3):657-666.) In some embodiments, the disclosed A. muscaria formulations are used to treat a sexual disorder, such as a disorder characterized by sexual dysfunction. In embodiments, the disclosed A. muscaria formulations are used in the manufacture of a medicament to treat a sexual disorder, such as a disorder characterized by sexual dysfunction. In embodiments, the disclosed A. muscaria formulations, e.g., in a therapeutically effective amount, are administered to a subject having a sexual disorder to treat said sexual disorder. Sexual dysfunctions can be defined as syndromes wherein a subject may have difficulty experiencing personally satisfying, non-coercive sexual activities. In embodiments, a disclosed formulation is used to treat hypoactive sexual desire dysfunction, sexual arousal dysfunction, orgasmic dysfunction, ejaculatory dysfunction, or sexual dysfunction associated with pelvic organ prolapse.

In some embodiments, the sexual disorder is delayed ejaculation and erectile disorder. In embodiments, the sexual disorder is female orgasmic disorder. In embodiments, the sexual disorder is female sexual interest or arousal disorder. In embodiments, the sexual disorder is genito-pelvic pain/penetration disorder. In embodiments, the sexual disorder is female hypoactive sexual desire disorder. In embodiments, the sexual disorder is male hypoactive sexual desire disorder. In embodiments, the sexual disorder is premature ejaculation. In embodiments, the sexual disorder is substance/medication-induced sexual dysfunction.

Signs or symptoms of sexual dysfunction can be assessed and alleviation thereof determined according to methods known to one of skill. Such evaluations include interviewing, use of assessments and questionnaires, and physiological investigations (Bhugra & Colombini, Advances in Psychiatric Treatment, 2018; 19(1): 48-55; Clayton, J Clin Psychiatry. 2001; 62 Suppl 3:5-9). In some embodiments, sexual function is assessed by evaluating, for example determining improvements in, one or more of sexual desire, sexual aversion, sexual enjoyment, sexual drive, genital response, such as vaginal dryness/impotence and male erectile disorder, orgasm disorders, such as orgasmic dysfunction and premature ejaculation, disorders of sexual pain, such as dyspareunia and vaginismus, and other forms of dysfunction.

The International Consultation on Sexual Medicine has proposed updated methods for diagnosing sexual dysfunction in men and women, with specific recommendations for sexual history taking and diagnostic evaluation. Standardized scales, checklists, and validated questionnaires are adjuncts that may be used to evaluate sexual dysfunction and sexual satisfaction (Hatzichristou et al., J Sex Med. 2016; 13(8):1166-8). Exemplary questionnaires include, for example, The Sexual Satisfaction Questionnaire, The Sexual Interest and Satisfaction Scale, The Sexual Satisfaction Scale, The Pinney Satisfaction Inventory, and the Female Sexuality Satisfaction Questionnaire

In some embodiments, the autoimmune disorder is a systemic autoimmune disorder, such as systemic lupus erythematosus (SLE), Sjogren's syndrome, scleroderma, rheumatoid arthritis, and polymyositis. In embodiments, the autoimmune disorder is a local autoimmune disorder, including those of the endocrine system, including type 1 diabetes, Hashimoto's thyroiditis, and Addison's disease; the cutaneous, including pemphigus vulgaris; the blood, including autoimmune hemolytic anemia; and the nervous system, including multiple sclerosis.

### G. ADDITIONAL EXAMPLES

### Example 20: Administration of a Disclosed Vaporizable Formulation to Enhance Calmness

An individual is administered or self-administers in a single day a suitable amount of a disclosed vaporizable formulation comprising an Amanita muscaria extract. The formulation is administered by inhalation, for example using a vaporizer device as disclosed herein or otherwise known in the art.

Post-administration, the individual experiences enhanced calmness compared to the level of calmness experienced prior to administration, as measured by, e.g., an assessment completed by the individual prior to, and after administration.

### Example 21: Administration of Disclosed Vaporizable Formulation to Reduce Muscle Tension

An individual is administered or self-administers in a single day a suitable amount of a disclosed vaporizable formulation comprising an Amanita muscaria extract. The formulation is administered by inhalation, for example using a vaporizer device as disclosed herein or otherwise known in the art.

Post-administration, the individual experiences reduced muscle tension compared to the level of muscle tension experienced prior to administration of the formulation, as measured by, e.g., an assessment completed by the individual prior to, and after such application. As an example, an individual may rate their muscle tension on a scale of 0-10 prior to, and after administering the formulation, wherein “0” represents an absence of muscle tension, and “10” represents severe muscle tension that restricts movement. In such an example, administration of the formulation will result in a reduced muscle tension rating.

As another example, an individual may measure their range of motion prior to, and after administering the formulation. In such an example, the individual will experience increased range of motion after administering the formulation.

### Example 22: Administration of a Disclosed Vaporizable Formulation to Reduce Insomnia

An individual is administered or self-administers in a single day a suitable amount of a disclosed vaporizable formulation comprising an Amanita muscaria extract. The formulation is administered by inhalation, for example using a vaporizer device as disclosed herein or otherwise known in the art.

Post-administration, the individual experiences reduced insomnia compared to the severity of insomnia prior to administering the beverage powder or ready to drink beverage. For example, the individual will experience the ability to, for example, fall asleep faster, stay asleep longer once asleep, and awake feeling more restful. This may be measured by, for example, a sleep assessment completed without ingesting, and after ingesting the beverage powder or ready to drink beverage. Example assessments include, for example, the insomnia severity index. In such an example, the Insomnia Severity Index (Insomnia severity index—ons.org) will decrease in score after ingesting the A. muscaria formulation.

### Example 23: Administration of a Disclosed Vaporizable Formulation to Treat a Medical Condition

An individual is administered or self-administers in a single day a suitable amount of a disclosed vaporizable formulation comprising an Amanita muscaria extract. The formulation is administered by inhalation, for example using a vaporizer device as disclosed herein or otherwise known in the art.

Post-administration, the individual experiences a reduction in signs or symptoms of the medical condition; relief from the condition or of a symptom or pathology caused by or related to the condition; a reduction, decrease, inhibition, or amelioration of the severity, duration, progression, frequency, or probability of one or more symptoms or pathologies associated with the condition; or a prevention or inhibition of a worsening or progression of symptoms or pathologies associated with the condition; as compared with prior to administration, or another such measure of treatment of the medical condition, as measured by, e.g., an assessment completed by the individual prior to, and after administration. In some embodiments, the medical condition is a mental, behavioral, or neurodevelopmental disorder. In some embodiments, the mental, behavioral, or neurodevelopmental disorder is a disorder as disclosed herein.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

The invention claimed is:

1. A vaporizable formulation comprising:
a. an Amanita muscaria extract;
b. a base liquid; and
c. optionally, a flavorant;
wherein the mass ratio of muscimol:ibotenic acid in the Amanita muscaria extract is greater than about 10:1.

2. The vaporizable formulation of claim 1, wherein the muscimol:ibotenic acid ratio is greater than about 100:1.

3. The vaporizable formulation of claim 1, wherein the muscimol:ibotenic acid ratio is greater than about 1000:1.

4. The vaporizable formulation of claim 1, wherein the muscimol:ibotenic acid ratio is between about 10:1 and 50:1.

5. The vaporizable formulation of claim 1, wherein the muscimol:ibotenic acid ratio is between about 10:1 and 30:1.

6. The vaporizable formulation of claim 1, wherein the muscimol:ibotenic acid ratio is about 12.5:1.

7. The vaporizable formulation of claim 1, wherein the muscimol:ibotenic acid ratio is about 25:1.

8. The vaporizable formulation of claim 1, wherein the Amanita muscaria extract comprises less than about 550 μg/g of muscarine.

9. The vaporizable formulation of claim 1, wherein the Amanita muscaria extract comprises less than about 40 μg/g or about 20 μg/g of stizolobinic acid.

10. The vaporizable formulation of claim 1, wherein the Amanita muscaria extract comprises less than about 0.05 ppm, about 0.04 ppm, or about 0.03 ppm of arsenic.

11. The vaporizable formulation of claim 1, wherein the Amanita muscaria extract comprises less than about 0.30 ppm, about 0.25 ppm, about 0.20 ppm, about 0.15 ppm, about 0.10 ppm, or about 0.09 ppm of cadmium.

12. The vaporizable formulation of claim 1, wherein the Amanita muscaria extract comprises less than about 0.15 ppm, about 0.10 ppm, about 0.05 ppm, or about 0.02 ppm of mercury.

13. The vaporizable formulation of claim 1, wherein the Amanita muscaria extract comprises less than about 0.09 ppm, about 0.05 ppm, about 0.04 ppm, about 0.03 ppm, or about 0.02 ppm of lead.

14. The vaporizable formulation of claim 1, produced by:
a. suspending an Amanita muscaria extract in a solvent;
b. mixing the suspension;
c. isolating the supernatant from the suspension, thereby producing an extract solution;
d. evaporating the extract solution to provide a residue; and
e. dissolving the residue in a base liquid to produce the vaporizable formulation.

15. The vaporizable formulation of claim 14, wherein the Amanita muscaria extract is a ground powder extract.

16. The vaporizable formulation of claim 14, wherein the solvent is an organic solvent.

17. The vaporizable formulation of claim 14, wherein the solvent is a methanol.

18. The vaporizable formulation of claim 14, wherein isolating the supernatant comprises centrifuging of the suspension.

19. The vaporizable formulation of claim 14, wherein evaporating the extract solution comprises heating the extract solution.

20. The vaporizable formulation of claim 1, produced by:
a. performing an aqueous extraction of Amanita muscaria mushroom biomass in the presence of heat to produce the extract;
b. reducing the pH of the extract;
c. concentrating the extract; and
d. dissolving the extract in a base liquid to produce the vaporizable formulation.

21. The vaporizable formulation of claim 20, wherein the aqueous extraction is performed in water heated to between about 70° C. and about 100° C.

22. The vaporizable formulation of claim 20, wherein the aqueous extraction is performed in water heated to between about 80° C.

23. The vaporizable formulation of claim 20, wherein the pH is reduced to between about 2.5 and about 3.0.

24. The vaporizable formulation of claim 1, wherein the base liquid comprises propylene glycol, vegetable glycerin, or a combination thereof.

25. The vaporizable formulation of claim 1, wherein the mass ratio of the Amanita muscaria extract to the base liquid is about 1:10 extract:base liquid (w/v).

26. The vaporizable formulation of claim 1, comprising between about 40% v/v and about 90% v/v of the base liquid.

27. The vaporizable formulation of claim 1, comprising between about 40% v/v and about 90% v/v of propylene glycol.

28. The vaporizable formulation of claim 1, comprising between about 40% v/v and about 90% v/v of vegetable glycerin.

29. The vaporizable formulation of claim 1, comprising between about 0.1% v/v and about 10% v/v of the flavorant.

30. The vaporizable formulation of claim 1, comprising between about 0.1% v/v and about 5% v/v of the flavorant.

31. The vaporizable formulation of claim 1, comprising between about 0.1% v/v and about 1% v/v of the flavorant.

32. A method of producing the vaporizable formulation of any one of claims 1-31, comprising:
a. suspending an Amanita muscaria extract in a solvent;
b. mixing the suspension;
c. isolating the supernatant from the suspension, thereby producing an extract solution;
d. evaporating the extract solution to provide a residue; and
e. dissolving the residue in a base liquid to produce the vaporizable formulation.

33. The method of claim 32, wherein the Amanita muscaria extract is a ground powder extract.

34. The method of claim 32, wherein the solvent is an organic solvent.

35. The method of claim 32, wherein the solvent is a methanol.

36. The method of claim 32, wherein isolating the supernatant comprises centrifuging of the suspension.

37. The method of claim 32, wherein evaporating the extract solution comprises heating the extract solution.

38. The method of claim 32, further comprising dissolving a flavorant in the base liquid.

39. A method of producing the vaporizable formulation of any one of claims 1-31, comprising:
a. performing an aqueous extraction of Amanita muscaria mushroom biomass in the presence of heat to produce the extract;
b. reducing the pH of the extract;
c. concentrating the extract; and
d. dissolving the extract in a base liquid to produce the vaporizable formulation.

40. The method of claim 39, wherein the aqueous extraction is performed in water heated to between about 70° C. and about 100° C.

41. The method of claim 39, wherein the aqueous extraction is performed in water heated to about 80° C.

42. The method of claim 39, wherein the pH is reduced to between about 2.0 and about 4.0.

43. The method of claim 39, wherein the pH is reduced to between about 2.5 and about 3.0.

44. The method of claim 39, further comprising dissolving a flavorant in the base liquid.

45. A method of modulating neurotransmission in a subject, comprising administering to the subject the vaporizable formulation of any one of claims 1-31.

46. The method of claim 45, wherein modulating neurotransmission comprises modulating one or more of gabaminergic, glutaminergic, and cholinergic neurotransmission.

47. The vaporizable formulation of any one of claims 1-31, for use in modulating neurotransmission.

48. Use of the vaporizable formulation of claim 47, wherein modulating neurotransmission comprises modulating one or more of gabaminergic, glutaminergic, and cholinergic neurotransmission.

49. Use of the vaporizable formulation of any one of claims 1-31, for the manufacture of a medicament for the treatment of a medical condition.

50. A method of treating a medical condition in a subject in need of such treatment, comprising administering to the subject the vaporizable formulation of any one of claims 1-31.

51. The method of claim 50, wherein the medical condition is a disorder linked to dysregulation or inadequate functioning of gabaminergic, glutaminergic, or cholinergic neurotransmission.

52. The method of claim 50, wherein the medical condition is a mental, behavioral, or neurodevelopmental disorder.

53. The method of claim 52, wherein the mental, behavioral, or neurodevelopmental disorder is a neurodevelopmental disorder, schizophrenia or another primary psychotic disorder, a mood disorder, an anxiety or fear-related disorders, an obsessive-compulsive or related disorder, a disorder specifically associated with stress, a feeding or eating disorder, a disorder of bodily distress or bodily experience, a disorder due to substance use or addictive behavior, an impulse control disorder, a disruptive behavior or dissocial disorder, a personality disorder, a paraphilic disorder, a neurocognitive disorder, a sleep-wake disorder, or a sexual dysfunction.

54. The method of claim 50, wherein the medical condition is pain or a pain disorder, inflammation or an inflammatory disorder, an immune or autoimmune disorder, or a sleep disorder.

55. The method of claim 54, wherein the pain disorder is any of arthritis, allodynia, atypical trigeminal neuralgia, trigeminal neuralgia, somatoform disorder, hypoesthesis, hyperalgesia, neuralgia, heuritis, neurogenic pain, analgesia, anesthesia dolorosa, causalgia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer.

56. The method of claim 54, wherein the immune or autoimmune disorder is any of acute disseminated encephalomyelitis, Addison disease, allergy or hypersensitivity, amyotrophic lateral sclerosis, antiphospholipid antibody syndrome, arthritis, autoimmune hemolysis anemia, autoimmune hepatitis, autoimmune inner ear disease, autoimmune pancreatitis, bullous pemphigoid, celiac disease, Chagas disease, chronic obstructive pulmonary disease, type 1 diabetes, endometriosis, fibromyalgia, goodpasture's syndrome, Graves' disease, Guillain-Barre syndrome, Hashimoto's thyroiditis, suppurative spondylitis, idiopathic thrombocytopenia purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, systemic lupus erythematosus, morphea, multiple sclerosis, myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, vitiligo, a systemic autoimmune disorder, systemic lupus erythematosus, Sjogren's syndrome, scleroderma, rheumatoid arthritis, and polymyositis.

57. The method of claim 54, wherein the sleep disorder is insomnia, hypersomnia, parasomnia, or disorder of sleep-wake schedule.

58. A method of improving health and wellness in a subject, comprising administering to the subject the vaporizable formulation of any one of claims 1-31.

59. The method of claim 58, wherein improving health and wellness comprises any of reducing stress, easing muscular tension, promoting restorative sleep, soothing the body, calming the mind, reducing physical distress, reducing anxiety, and inducing euphoria.

60. The method of claim 58, wherein improving health and wellness comprises treating a medical condition.

61. A vaporizer device, comprising:
a. a mouthpiece;
b. a heating element;
c. a power source; and
d. a chamber comprising the vaporizable formulation of any one of claims 1-31.

62. The vaporizer device of claim 61, wherein the heating element is capable of heating the vaporizable formulation to a set temperature.

63. The vaporizer device of claim 62, wherein the set temperature is sufficient to vaporize the vaporizable formulation.

64. The vaporizer device of claim 61, wherein the power source powers the heating element.

65. The vaporizer device of claim 61, wherein the heating element comprises a wick element.

66. The vaporizer device of claim 61, wherein the chamber comprises a first end configured to attach to the mouthpiece.

67. The vaporizer device of claim 61, wherein the chamber comprises a second end configured to attach to the heating element and the power source.

68. The vaporizer device of claim 61, wherein the chamber is refillable.

69. The vaporizer device of claim 61, wherein the chamber is removable.

70. The vaporizer device of claim 61, further comprising a means for controlling the device.

71. The vaporizer device of claim 70, wherein the means for controlling the device comprises switches, buttons, touch sensors, or a combination thereof.

72. The vaporizer device of claim 61, further comprising a means for displaying information.

73. The vaporizer device of claim 72, wherein the means for displaying information comprises a screen, a number of lights, or a combination thereof.

74. The vaporizer device of claim 61, further comprising a number of sensors configured to sense temperature, pH, volume, the means for controlling the device, or a combination thereof.
